(19) United States
(12) Patent Application Publication
(10) Pub. No.: US 2019/0295726 A1
(43) Pub. Date: Sep. 26, 2019

(54) SYSTEMS AND METHODS FOR MONITORING SUBJECTS FOR HEREDITARY CANCERS

(71) Applicant: GenID Solutions, LLC, Santa Clara, CA (US)

(72) Inventor: Anita P. Singh, Santa Clara, CA (US)

(21) Appl. No.: 16/359,927

(22) Filed: Mar. 20, 2019

Related U.S. Application Data
(60) Provisional application No. 62/645,660, filed on Mar. 20, 2018.

(51) Int. Cl. G16H 50/30; G16B 40/00; G16H 10/60

(52) U.S. Cl. CPC G16H 50/30; G16H 10/60; G16B 40/00

(57) ABSTRACT

Systems and methods for monitoring subjects for a variety of hereditary cancers include obtaining an electronic medical record associated with a subject. Data items are extracted from the medical record of the subject. The data items describe information of the electronic medical record and include at least an age of the subject as well as a cancer history of family of the subject. The data items are run against various filter sets. Each filter set is associated with a different hereditary cancer and has a corresponding alert. When a filter is fired, due to triggering the conditions of the filter, the subject is deemed to have an actionable risk of the hereditary cancer associated with the corresponding filter set. The alert, that includes the filter that is fired, is communicated accordingly.

### CROSS REFERENCE TO RELATED APPLICATION

This application claims priority to U.S. Provisional Application No. 62/645,660 entitled “Systems and Methods for Monitoring Subjects for Hereditary Cancers,” filed Mar. 20, 2018, which is hereby incorporated by reference.

### TECHNICAL FIELD

The present disclosure relates generally to systems and methods for monitoring subjects for hereditary diseases, e.g., by determining whether a first human has an actionable risk for each of a plurality of hereditary cancers. More particularly, the present disclosure relates generally to systems and methods for monitoring a subject for different hereditary cancers utilizing medical information from persons that the subject shares a consanguineous relationship.

### BACKGROUND

Adoption of the Health Information Technology for Economic and Clinical Health (HITECH) Act of 2009 has led to widespread use of electronic medical records (EMR), also known as electronic health records (HER). EMRs are configured to replace physical paper records with an electronic collection of personal health records, and are considered to be a significant tool for individualized disease prevention, diagnosis, and treatment.

EMRs include a family medical history of a subject, track data related to the subject over extended time periods, and monitor the subject and their relative physiological wellbeing as compared to known standards, such as vaccination rates or blood pressure readings. The family medical history portion of the EMR is utilized for collecting information related to the medical history and living status of various family members of the subject, such as which family members have been diagnosed with a disease or are susceptible to a cancer.

According to a recent study by the American Cancer Society, approximately five to ten percent of all cancers are hereditary cancers. Hereditary cancers are cancers that formed when a subject has inherited a cancer susceptibility gene from their family lineage. Of persons with genes susceptible to cancer, there is up to an eighty percent lifetime risk of cancer. Thus, EMRs are a valuable source of data pertaining to plausible familial risk factors for hereditary cancers (Chen et al., 2012, “Characterizing the Use and Contents of Free-Text Family History Comments in the Electronic Health Record,” AMIA Annu Symp Proc, p. 85).

Knowledge discovery and data mining have the potential to transform EMR data into actionable disease knowledge. The collection of family history data in EMR systems provides an opportunity for validating known associations and discovering novel interactions among familial risk factors and hereditary diseases (Chen et al., 2015, “Mining and Visualizing Family History Associations in the Electronic Health Record: A Case Study for Pediatric Asthma,” AMIA Annu Symp Proc., p. 396). However, significant challenges exist in extracting the data. These challenges include multiple data entry formats in a typical EMR, including structured and free-text forms, limited practitioner awareness and information, as well as time constraints of clinicians such as an inability to analyze the full family medical history of each subject. (Chen et al., 2012, “Characterizing the Use and Contents of Free-Text Family History Comments in the Electronic Health Record,” AMIA Annu Synp Proc, p. 85). For instance, according to a recent study, over one-half of the available time of a practitioner in a workday is spent interacting with EMRs (Arndt et al., 2017, “Tethered to the EHR: Primary Care Physician Workload Assessment Using HER Event Log data and Time-Motion Observations,” Ann. Fam. Med. 15, p. 419). Health care providers often cannot stay up-to-date with emerging genetic research in order to provide their patients and subjects with state of the art care and preventative medicine

The 2009 final statement of the National Institute of Health (NIH) called for meaningful outcomes of family history acquisition, interpretation, and application (Berg et al., 2009, “National Institutes of Health State-of-the-Science Conference Statement: Family History and Improving Health,” Ann Intern Med. 12, p. 872).

However, there are disadvantages to genetic testing, as well. For example, genetic testing can cause anxiety and stress, genetic testing is expensive, and even favorable outcomes from genetic testing do not reduce the risk of cancer. Moreover, genetic testing raises privacy and discriminatory concerns, both from a socioeconomic and healthcare/insurance perspectives. This, in turn, can lead to additional anxiety and stress. Accordingly, it is not feasible or desirable to genetically screen every person for increased cancer risks.

The information disclosed in this Background section is only for enhancement of understanding of the general background of the invention and should not be taken as an acknowledgement or any form of suggestion that this information forms the prior art already known to a person skilled in the art.

### SUMMARY

Given the above background, improved methods and systems are needed for economical extraction and efficient parsing of family medical history data relating to hereditary cancer risks, e.g., from EMRs. Particularly, these methods and systems should not increase the time burdens of health care staff and, ideally, actually reduce those burdens. Moreover, improved methods for appropriately selecting individuals for genetic testing are needed. Advantageously, the systems and methods for monitoring subjects for hereditary cancers detailed in the present disclosure address these and other shortcomings.

For instance, in some aspects, the methods and systems described herein leverage novel algorithms that extract relevant medical records, e.g., from EMRs, and apply those records to a series of filters that efficiently selects those patients who would benefit most from genetic screening, while reducing the burden on medical professionals and avoiding unnecessary genetic screening. In some embodiments, the novel algorithms described herein evaluate actionable hereditary cancer risks based on, at least, the identification of familial risk factors that meet a threshold level of consanguinity. In contrast, conventional methods for identifying patients for genetic screening do not use such algorithms, and rely more on the discretion of the patient, to determine whether genetic testing will be performed.

One aspect of the present disclosure provides methods, systems, and non-transitory computer readable storage medium for implementing monitoring of subjects for hereditary cancers. For instance, in some embodiments, a non-transitory computer readable storage medium stores instructions, which when executed, in response to a performance of a medical procedure on a subject by a medical practitioner, causes a device to obtain an electronic medical record (EMR) associated with the subject after the electronic medical record has been updated to reflect the medical procedure. Responsive to this, data items pertaining to the subject are extracted from the electronic medical record of the subject. These data items include an age of the subject as well as an indication of whether another human associated with the subject has been afflicted with a cancer (e.g., a hereditary cancer). In some instances, the other human associated with the subject shares a first degree, second degree, or third degree consanguineous relationship with the subject. For each respective hereditary cancer in a predetermined set of hereditary cancers, all or a portion of the data items retrieved from the subject are run against a corresponding filter set in a plurality of filter sets. Each respective filter set in the plurality of filter sets represents a different hereditary cancer. Furthermore, each respective filter set in the plurality of filter sets is associated with a corresponding alert in a plurality of alerts. When a respective filter in the corresponding filter set is fired, meaning that the query conditions or test conditions of the filter have been satisfied by or identified in the retrieved data, the subject is deemed to have an actionable risk of the hereditary cancer represented by the corresponding filter set. The non-transitory computer readable storage medium further stores instructions for communicating the alert associated with each respective filter set in the plurality of filter sets that includes a filter that have been fired.

In one aspect, the disclosure provides methods, systems, and computer non-transitory computer readable storage medium for determining whether a first human has an actionable risk for each of a plurality of hereditary cancers. The methods include, responsive to a request by a medical practitioner, obtaining an electronic medical record associated with the first human via a computer system having a processor programmed to receive medical records. The electronic medical record of the first human including a plurality of data items, wherein the plurality of data items includes an age of the first human, whether the first human has been diagnosed with a cancer, and an indication of whether a second human has been afflicted with a cancer, where the second human shares a first degree, second degree, or third degree consanguineous relationship with the first human. For each respective hereditary cancer in the plurality of hereditary cancers, the method includes applying an algorithm to the plurality of data items, via a computer system having a processor programmed to perform the algorithm, where the algorithm runs the plurality of data items against a corresponding filter set in a plurality of filter sets. Each respective filter set in the plurality of filter sets represents a different hereditary cancer in the plurality of hereditary cancers. Each respective filter set in the plurality of filter sets includes a respective filter that is configured to be fired at least when the plurality of data items indicates the first human has a familial risk factor for the corresponding hereditary cancer that satisfies a first threshold level of consanguinity. Each respective filter set in the plurality of filter sets is associated with a corresponding alert in a plurality of alerts. When a respective filter in the corresponding filter set is fired, the first human is deemed to have an actionable risk of the hereditary cancer represented by the corresponding filter set. The method then includes communicating a report to the medical professional, via a computer system having a processor programmed to communicate reports, the report comprising the alert associated with each respective filter set in the plurality of filter sets that included a respective filter that was fired in response to applying the algorithm to the plurality of data items of the electronic medical record of the first human. In some embodiments, the method also includes screening the human for a genetic predisposition to a hereditary cancer. In some embodiments, the method also includes, taking action in response to detecting a genetic predisposition to a hereditary cancer.

The systems and methods of the present disclosure have other features and advantages that will be apparent from, or are set forth in more detail in, the accompanying drawings, which are incorporated herein, and the following Detailed Description, which together serve to explain certain principles of exemplary embodiments of the present invention

### BRIEF DESCRIPTION OF THE DRAWINGS

FIG. 1 illustrates an exemplary system topology that includes an electronic medical record system for storing a plurality of medical records, a data parsing device for obtaining and parsing data items in the medical record, and medical practitioner devices associated with corresponding medical practitioners, where the above-identified components are interconnected, optionally through a communications network, in accordance with an embodiment of the present disclosure;

FIG. 2 illustrates an electronic medical record system for storing and communicating a plurality of medical records in accordance with an embodiment of the present disclosure;

FIG. 3 illustrates a data parsing device for obtaining and parsing medical record data in accordance with an embodiment of the present disclosure;

FIG. 4 illustrates a medical practitioner device for editing and receiving a medical record in accordance with an embodiment of the present disclosure;

FIG. 5 illustrates a general workflow for electronic medical record parsing in accordance with an embodiment of the present disclosure;

FIGS. 6A, 6B, 6C, 6D, 6E, 6F, 6G, 6H, 6I, and 6J collectively illustrate a flow chart of methods for monitoring subjects for hereditary cancers in accordance with an embodiment of the present disclosure, in which optional steps or embodiments are indicated by dashed boxes;

FIGS. 7A, 7B, 7C, 7D, and 7E illustrate an example code for API calls to an electronic medical records system in accordance with an embodiment of the present disclosure; and

FIGS. 8A, 8B, 8C, 8D, 8E, and 8F illustrate example codes for a variety of filters and a decision engine in accordance with an embodiment of the present disclosure.

FIG. 9 illustrates an example method for determining whether a first human has an actionable risk for each of a plurality of hereditary cancers, in accordance with some embodiments of the present disclosure.

The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Reference will now be made in detail to implementations, examples of which are illustrated in the accompanying drawings. In the following detailed description of implementations, numerous specific details are set forth in order to provide a thorough understanding of the present invention. However, it will be apparent to one of ordinary skill in the art that the present invention may be practiced without these specific details.

It will also be understood that, although the terms first, second, etc. may be used herein to describe various elements, these elements should not be limited by these terms. These terms are only used to distinguish one element from another. For example, a first filter could be termed a second filter, and, similarly, a second filter could be termed a first filter, without departing from the scope of the present disclosure. The first filter and the second filter are both filters, but they are not the same filter. Furthermore, the terms “subject,” “first human,” and “patient” are used interchangeably herein. Additionally, the terms “clinician,” “physician,” and “practitioner” are used interchangeably herein.

The terminology used in the present disclosure is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

In the interest of clarity, not all of the routine features of the implementations described herein are shown and described. It will be appreciated that, in the development of any such actual implementation, numerous implementation-specific decisions are made in order to achieve the specific goals of the design, such as compliance with use case- and business-related constraints, and that these specific goals will vary from one implementation to another and from one design to another. Moreover, it will be appreciated that such a design effort might be complex and time-consuming, but nevertheless be a routine undertaking of engineering for those of ordinary skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

In the present disclosure, various cancers and conditions are defined by their Systematized Nomenclature of Medicine-Clinical Terms (SNOMED-CT) values. Incorporated by reference is browser.ihtsdotools.org which provides listings of SNOMED-CT values. In some embodiments, SNOMED-CT values are in accordance with the March 2018 SNOMED CT United States (US) Edition release available on the Internet at nlm.nih.gov/healthit/snomedct/us_edition.html as of Mar. 17, 2018, distributed by the United States National Library of Medicine, which is hereby incorporated by reference. Examples of these SNOMED-CT values include, but are not limited to, age {397659008}, whether or not the subject is deceased {419099009), and a dependency of the subject, e.g., Ashkenazi Jewish {81706006, 15086000, 315237009}.

As used herein, the terms “consanguinity” and “consanguineous relationship” refer to relationships in which subjects share a significant portion of their autosomal genetic heritage, as opposed to traditional notions of consanguinity, which refer to the degree of a familial relationship as shown in a Table of consanguinity. Accordingly, whereas siblings share approximately the same percentage of autosomal genetic heritage as do a parent-child relationship, as used herein, sibling relationships and parental relationships are considered to have a same degree of consanguinity. This is in contrast with conventional notions of consanguinity, where the parental relationship is a first order consanguineous relationship and siblings share a second order consanguineous relationship.

As used herein, the term “first degree consanguineous relationship,” refers to a familial relationship in which two subjects share approximately 50% of the autosomal portion of their genetic heritage. Examples of relatives of a subject that share a first degree consanguineous relationship include a biological parent, a biological sibling, and a biological child.

As used herein, the term “second degree consanguineous relationship,” refers to a familial relationship in which two subjects share approximately 25% of the autosomal portion of their genetic heritage. Examples of relatives of a subject that share a second degree consanguineous relationship include a biological grandparent, a biological aunt, a biological uncle, a biological half-bother, a biological half-sister, a biological nephew, and a biological niece.

As used herein, the term “third degree consanguineous relationship,” refers to a familial relationship in which two subjects share from approximately 1% to approximately 12.5% of the autosomal portion of their genetic heritage. Examples of relatives of a subject that share a third degree consanguineous relationship include biological great grandparents, great nephews and nieces, cousins (e.g., first, second, third, and fourth cousins), etc.

In some embodiments, the systems and methods described herein for monitoring subjects for a variety of hereditary cancers include obtaining an electronic medical record associated with a subject. Data items are extracted from the electronic medical record of the subject. The data items typically include at least an age of the subject and an indication of whether a second consanguineous person (e.g., a blood related family member) has been afflicted with a cancer. The data items are run against a variety of filter sets. Each filter set has a corresponding alert and is associated a different hereditary cancer (e.g., filter set 218-1 is associated with colon cancer while filter set 218-2 is associated with breast cancer). When a filter is fired, the subject is deemed to have an actionable risk of the hereditary cancer associated with the corresponding filter set. The alert is communicated, which includes an indication of the corresponding fired filter.

Example System Topologies

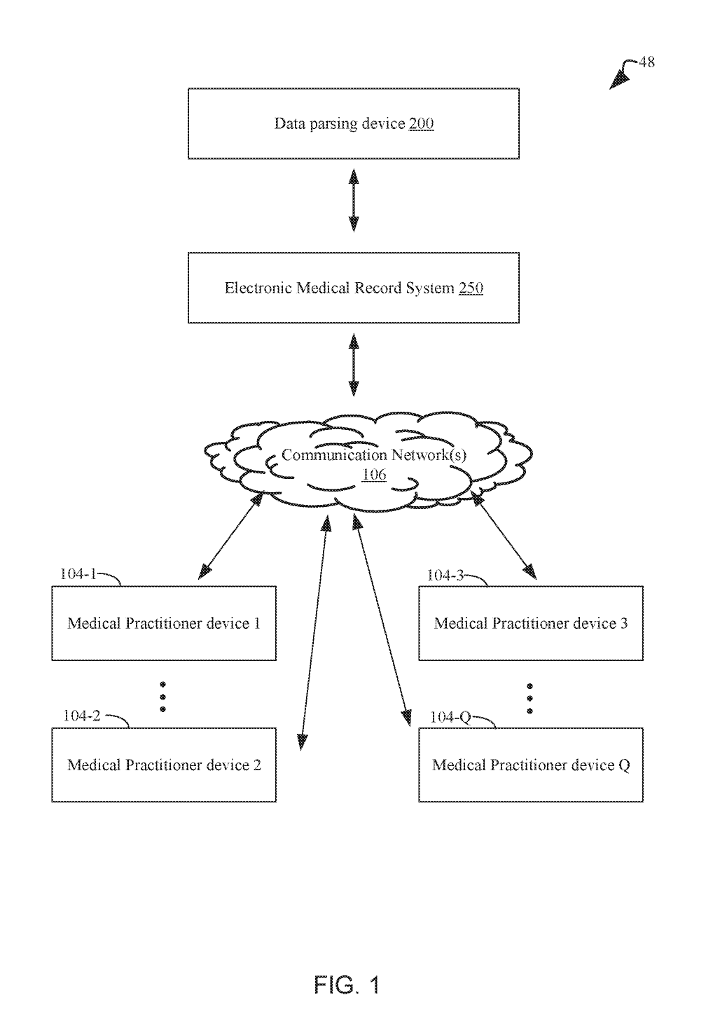

A detailed description of a system 48 for monitoring subjects for hereditary cancers in accordance with the present disclosure is detailed in conjunction with FIGS. 1 through 4. As such, FIGS. 1 through 4 collectively illustrate a topology of the system in accordance with some embodiments of the present disclosure. In the topology, there is an electronic medical record (EMR) system, or registry, 250 configured for receiving and storing a plurality of medical records from various medical practitioner devices 104. The system also includes a data parsing device 200 configured to communicate with the electronic medical record system 250. Throughout the present disclosure, the data parsing device 200 and the electronic medical record system 250 will be referenced as separate devices solely for the purpose of clarity. That is, the disclosed functionality of the data parsing device 200 and the disclosed functionality of the electronic medical record system 250 are contained in separate devices as illustrated in FIG. 1. However, it will be appreciated that, in fact, in some embodiments, the disclosed functionality of data parsing device 200 and the disclosed functionality of electronic medical record system 250 are contained in a single device.

Although not depicted in FIG. 1, in typical embodiments where the data parsing device 200 is not subsumed by the electronic medical record system 250, there is a single medical practitioner device 104 for each medical practitioner, and thus there typically exists a one-to-many relationship between the data parsing device 200 and the medical practitioner devices 104. Moreover, in some embodiments, there is a data parsing device 200 subsumed with each respective medical practitioner device 104, and thus there exists a one-to-one relationship between the data parsing device 200 and each medical practitioner device 104.

Referring to FIG. 1, the electronic medical record system 250 is configured to store a plurality of medical records. To do this, electronic medical record system 250, which is in communication with the medical practitioner devices 104, receives data elements originating from one or more medical practitioner devices 104 that have been provided to a registered medical practitioner. Each such data elements comprises a portion of a medical record of a subject of the medical practitioner. In some embodiments, the EMR system 250 communicates data elements wirelessly through radio-frequency (RF) signals. In some embodiments, such signals are in accordance with 802.11 (Wi-Fi), Bluetooth, or ZigBee standard. In some embodiments, the data parsing device 200 receives data elements from the EMR system 250, parses the data elements, and communicates the parsed data elements to the EMR system 250. In some embodiments, the data parsing device 200 receives data elements from the EMR system 250, parses the data elements, and communicates the parsed data elements to the corresponding medical practitioner device(s) 104.

In some embodiments, the data parsing device 200 and/or the EMR system 250 is not proximate to the medical practitioner devices 104 and/or does not have wireless capabilities or such wireless capabilities are not used for the purpose of acquiring data elements. In such embodiments, a communication network 106 is used to communicate data elements from the medical devices 104 to/from the data parsing device 200 and/or the EMR system 250.

Examples of networks 106 include, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

Of course, other topologies of the system 48 other than the one depicted in FIG. 1 are possible. For example, in some instances, rather than relying on a communications network 106, the one or more medical practitioner devices 104 wirelessly transmit information directly to the EMR system 250. Furthermore, in some instances, the data parsing device 200 and/or the EMR system 250 constitute a portable electronic device, a server computer, or in fact constitute several computers that are linked together in a network or a virtual machine or container in a cloud computing context. As such, the exemplary topology shown in FIG. 1 merely serves to describe the features of an exemplary embodiment of the present disclosure in a manner that will be readily understood to one of skill in the art.

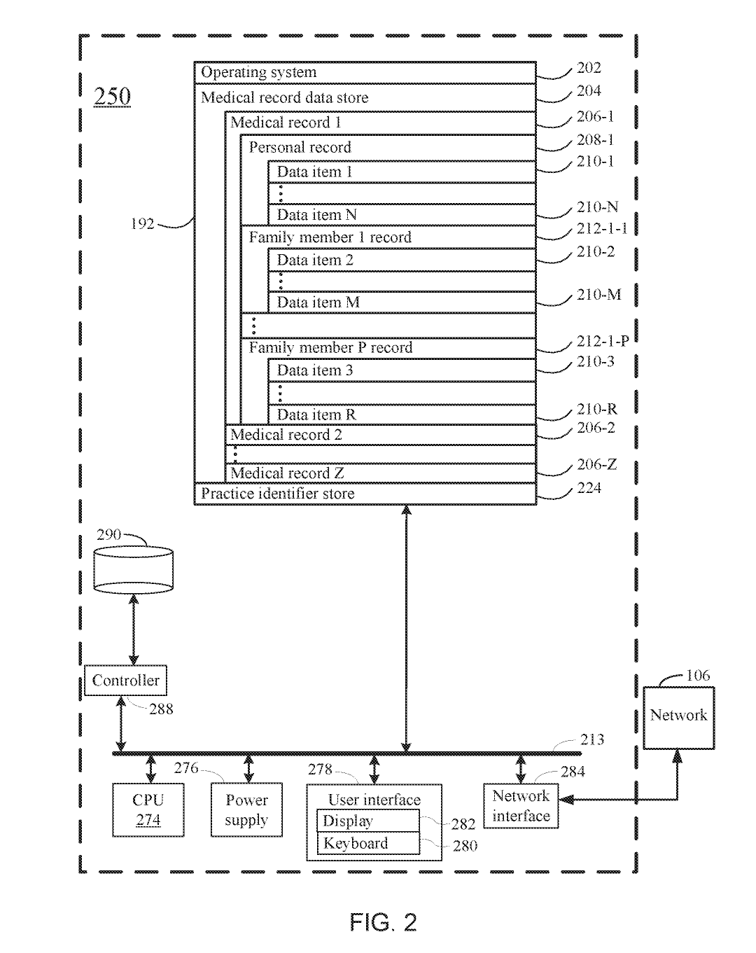

Referring to FIG. 2, in some embodiments, the EMR system 250 comprises one or more computers. For purposes of illustration in FIG. 2, the EMR system 250 is represented as a single computer that includes all of the functionality for monitoring subjects for hereditary cancers. However, the disclosure is not so limited. In some embodiments, the functionality for monitoring subjects for hereditary cancers is spread across any number of networked computers and/or resides on each of several networked computers and/or is hosted on one or more virtual machines at a remote location accessible across the communications network 106. One of skill in the art will appreciate that any of a wide array of different computer topologies are used for the application and all such topologies are within the scope of the present disclosure.

Turning to FIG. 2 with the foregoing in mind, an exemplary EMR system 250 for storing a plurality of medical records comprises one or more processing units (CPUs) 274, a network or other communications interface 284, a memory 192 (e.g., random access memory), one or more magnetic disk storage and/or persistent devices 290 optionally accessed by one or more controllers 288, one or more communication busses 213 for interconnecting the aforementioned components, a user interface 278, the user interface 278 including a display 282 and input 280 (e.g., keyboard, keypad, touch screen), and a power supply 276 for powering the aforementioned components. In some embodiments, data in memory 192 is seamlessly shared with non-volatile memory 290 using known computing techniques such as caching. In some embodiments, memory 192 and/or memory 290 includes mass storage that is remotely located with respect to the central processing unit(s) 274. In other words, in such instances, some data stored in memory 192 and/or memory 290 is in fact hosted on computers that are external to the EMR system 250, but also electronically accessed by the EMR 250 over an Internet, intranet, or other form of network or electronic cable (illustrated as element 106 in FIG. 2) using network interface 284.

In some embodiments, the memory 192 of the EMR system 250 for storing medical records stores:
an operating system 202 that includes procedures for handling various basic system services;
a medical record data store 204 that stores a plurality of medical records 206, where each respective medical record 206-1 in the plurality of medical records 206 is for a corresponding subject and stores the personal medical record 208 of a subject and medical records of subjects 212 that are consanguineous to the subject, where each record 208, 212 includes data items 210, each data item 210 reflecting various medical data; and
a practice identifier (ID) store 224 that stores identifiers for various practices, each identifier being uniquely associated with a corresponding practice or practitioner.

In some implementations, one or more of the above identified data elements or modules of the EMR system 250 are stored in one or more of the previously described memory devices, and correspond to a set of instructions for performing a function described above. The above-identified data items, modules or programs (e.g., sets of instructions) need not be implemented as separate software programs, procedures or modules, and thus various subsets of these modules may be combined or otherwise re-arranged in various implementations. In some implementations, the memory 192 and/or 290 optionally stores a subset of the modules and data structures identified above. Furthermore, in some embodiments the memory 192 and/or 290 stores additional modules and data structures not described above.

The data items 210 illustrated in FIG. 2 include medical information related to a subject and their family medical history. For instance, in some embodiments, a data item 210 comprises physiological measurements of the subject taken by a medical practitioner during an appointment. Each physiological measurement includes a measurement value or reference value related to the wellbeing of the subject. In some embodiments, the physiological measurement is a body temperature of the subject (e.g., the subject has a core body temperature of 93.6° F.). In some embodiments, the physiological measurement is a measurement of activity of the subject (e.g., the subject is not physically active). For instance, in some embodiments, the data item 210 includes the age of subject, the age of a family member of the subject, a living status of the subject (e.g., living or deceased), a medical procedure history of the subject (e.g., the subject received a flu shot this date), and the like. In some embodiments, a person that has a consanguineous relation to the subject of a personal record 208 have personal medical records 208 of their own. For instance, in one implementation the family member record 212-1-P in the medical record 206-1 is a copy of, or link to, the medical record 206-P of the person.

In some embodiments, a data item 210 comprises text in free-text form (e.g., the subject has been diagnosed with breast cancer), structured form (e.g., a predetermined matrix or spreadsheet to input values or make selections from a drop down window), or a combination thereof. In some embodiments, the data item 210 comprises a plurality of photos (e.g., photos of the subject taken from a camera), images (e.g., x-ray images of the subject), videos (e.g., colonoscopy camera feed recordings), or a combination thereof.

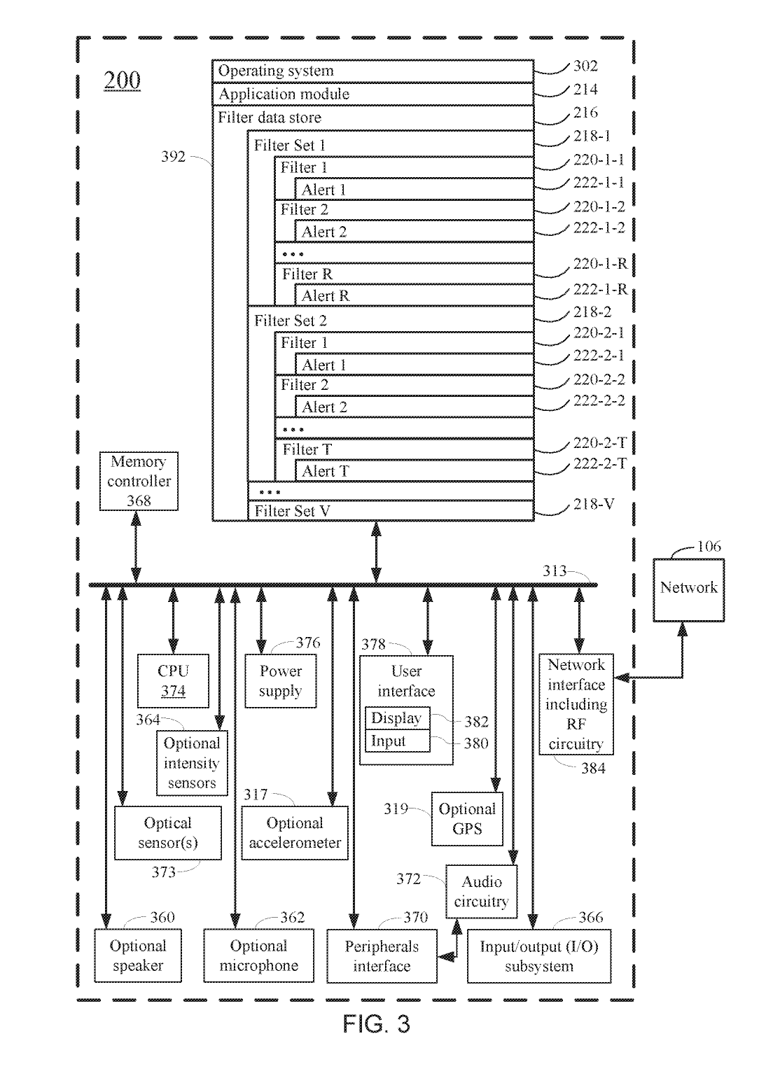

FIG. 3 illustrates an example data parsing device 200, in accordance with some embodiments of the present disclosure. The data parsing device 200 illustrated in FIG. 3 has one or more processing units (CPU's) 374, peripherals interface 370, memory controller 368, a network or other communications interface 384, a memory 392 (e.g., random access memory), a user interface 378, the user interface 378 including a display 382 and input 380 (e.g., keyboard, keypad, touch screen), an optional accelerometer 317, an optional GPS 319, optional audio circuitry 372, an optional speaker 360, an optional microphone 362, one or more optional intensity sensors 364 for detecting intensity of contacts on the data parsing device 200 (e.g., a touch-sensitive surface such as a touch-sensitive display system 382 of the data parsing device 200), an optional input/output (I/O) subsystem 366, one or more optional optical sensors 373, one or more communication busses 313 for interconnecting the aforementioned components, and a power supply 376 for powering the aforementioned components.

In some embodiments, the input 280 is a touch-sensitive display, such as a touch-sensitive surface. In some embodiments, the user interface 278 includes one or more soft keyboard embodiments. In some implementations, the soft keyboard embodiments include standard (QWERTY) and/or non-standard configurations of symbols on the displayed icons.

The data parsing device 200 illustrated in FIG. 3 optionally includes, in addition to accelerometer(s) 317, a magnetometer (not shown) and a GPS 319 (or GLONASS or other global navigation system) receiver for obtaining information concerning the location and orientation (e.g., portrait or landscape) of the data parsing device 200 and/or for determining an amount of physical exertion by the subject.

It should be appreciated that the data parsing device 200 illustrated in FIG. 3 is only one example of a multifunction device that may be used for parsing medical records 206 from the EMR system 250 and/or the medical device(s) 104 of a corresponding practice in a plurality of practices, and that the data parsing device 200 optionally has more or fewer components than shown, optionally combines two or more components, or optionally has a different configuration or arrangement of the components. In fact, as previously described, in some embodiments, the medical records 206 are acquired by the data parsing device 200 directly from the medical devices 104 without reliance on the EMR system 250.

The various components shown in FIG. 3 are implemented in hardware, software, firmware, or a combination thereof, including one or more signal processing and/or application specific integrated circuits.

Memory 392 of the data parsing device 200 illustrated in FIG. 3 optionally includes high-speed random access memory and optionally also includes nonvolatile memory, such as one or more magnetic disk storage devices, flash memory devices, or other non-volatile solid-state memory devices. Access to memory 392 by other components of the data parsing device 200, such as CPU(s) 274 is, optionally, controlled by the memory controller 368.

In some embodiments, the memory 392 of the data parsing device stores:
an operating system 302 that includes procedures for handling various basic system services;
an application module 214 for data parsing; and
a filter data store 216 that comprises a plurality of filter sets 218 and corresponding filters 220 and alerts 222.

In some such embodiments, the functionality of the instant application module 214 installed on the data parsing device 200 is limited to those that pertain to a corresponding single practitioner (e.g., single medical practitioner device 104) associated with the data parsing device 200. In many embodiments, the application module 214 is an instance of software utilized to implement methods of the present disclosure. In some embodiments, the application module 214 comprises an engine configured to run data items (e.g., data items 210 of FIG. 2) against a plurality of filter sets (e.g., filter sets 218 of FIG. 3). In such an implementation, the engine receives parsed data items 210 from the EMR system and returns an alert 220, or suggestion, to the EMR system of medical practitioner device(s) 104. In many embodiments, the engine is stored on a cloud service such as an elastic computer cloud (EC2 cloud).

In the present exemplary embodiment, the filters 220, or rules, of the engine is a decision tree classifier. However, the present disclosure is not limited thereto. Non-limiting examples of classifiers that may be used in conjunction with the present disclosure include a linear regression algorithm, a penalized linear regression algorithm, a non-linear regression algorithm, a support vector machine algorithm, an unsupervised clustering algorithm, a supervised clustering algorithm, a tree-based algorithm, a neural network, etc.

In some embodiments, each hereditary cancer in an enumerated set of hereditary cancers is associated with a filter set 218 in the filter data store 216. In some embodiments, there exists a one-to-one relationship between each filter set 218 and a corresponding hereditary cancer. However, the present disclosure is not limited thereto. In some embodiments, there exists a many-to-one relationship between the filter sets 218 and a corresponding hereditary cancer. In some embodiments, one or more filter sets 218 relates to a category of hereditary cancer, e.g., a hereditary non-polyposis colorectal cancer (HNPCC) related cancer or a hereditary breast and ovarian cancer syndrome (HBOC) related cancer.

Each respective filter set 218 comprises at least one filter 220. Each respective filter 220 specifies one or more alerts 222. The alerts 222 are activated when trigger conditions for the respective filter 220 are met, thereby firing the respective filter 220. For instance, in some embodiments a filter (e.g., filter 220-1 of FIG. 3) is configured to be run against the data items (e.g., data item 210-2, . . . , data item 210-M, data item 210-3, . . . , data item 210-R of FIG. 3) for two or more family members diagnosed with Lynch Syndrome of the subject. When the data items 210 trigger an indication that two or more family members of the subject have Lynch Syndrome, an alert 220 is fired which communicates the results of the filter to the medical practitioner device 104, the medical practitioner, and/or the subject “The subject has two or more family members with a history of Lynch Syndrome.”

In some embodiments, the peripherals interface 370 are used to couple input and output peripherals of the device to CPU(s) 374 and memory 392. The one or more processors 374 run or execute various software programs and/or sets of instructions stored in memory 392, such as the application module 214, to perform various functions for the data parsing device 200 and to process data.

In some embodiments, the peripherals interface 370, CPU(s) 374, and memory controller 368 are, optionally, implemented on a single chip. In some other embodiments, they are implemented on separate chips.

Radio frequency (RF) circuitry of network interface 384 receives and sends RF signals, also called electromagnetic signals. In some embodiments, the medical records 206 are received using this RF circuitry from one or more devices such as a medical device 104 associated with a corresponding practice. In typical embodiments, the medical records 206 are received using RF circuitry from the EMR system 250, as previously described. In some embodiments, the RF circuitry 384 converts electrical signals to/from electromagnetic signals and communicates with communications networks and other communications devices, medical devices 104 and/or the EMR system 250 via the electromagnetic signals. The RF circuitry 384 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 384 optionally communicates with the communication network 106. In some embodiments, the circuitry 384 does not include RF circuitry and, in fact, is connected to the network 106 through one or more hard wires (e.g., an optical cable, a coaxial cable, or the like).

In some embodiments, the audio circuitry 372, the optional speaker 360, and the optional microphone 362 provide an audio interface between the user and the data parsing device 200. The audio circuitry 372 receives audio data from the peripherals interface 370, converts the audio data to electrical signals, and transmits the electrical signals to the speaker 360. The speaker 360 converts the electrical signals to human-audible sound waves. The audio circuitry 372 also receives electrical signals converted by the microphone 362 from sound waves. The audio circuitry 372 converts the electrical signal to audio data and transmits the audio data to peripherals interface 370 for processing. Audio data is, optionally, retrieved from and/or transmitted to the memory 392 and/or the RF circuitry 384 by the peripherals interface 370.

In some embodiments, the power supply 376 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

In some embodiments, the data parsing device 200 optionally also includes one or more optical sensors 373. The optical sensor(s) 373 optionally include charge-coupled device (CCD) or complementary metal-oxide semiconductor (CMOS) phototransistors. The optical sensor(s) 373 receive light from the environment, projected through one or more lens, and converts the light to data representing an image. The optical sensor(s) 373 optionally capture still images and/or video.

As illustrated in FIG. 3, a data parsing device 200 preferably comprises an operating system 202 that includes procedures for handling various basic system services. The operating system 202 (e.g., iOS, DARWIN, RTXC, LINUX, UNIX, OS X, WINDOWS, or an embedded operating system such as Vx Works) includes various software components and/or drivers for controlling and managing general system tasks (e.g., memory management, storage device control, power management, etc.) and facilitates communication between various hardware and software components.

In some embodiments, the EMR system 250 has any or all of the circuitry, hardware components, and software components found in the parsing device 200 depicted in FIG. 3. In the interest of brevity and clarity, only a few of the possible components of the data parsing device 200 are shown in order to better emphasize the additional software modules that are installed on the data parsing device 200.

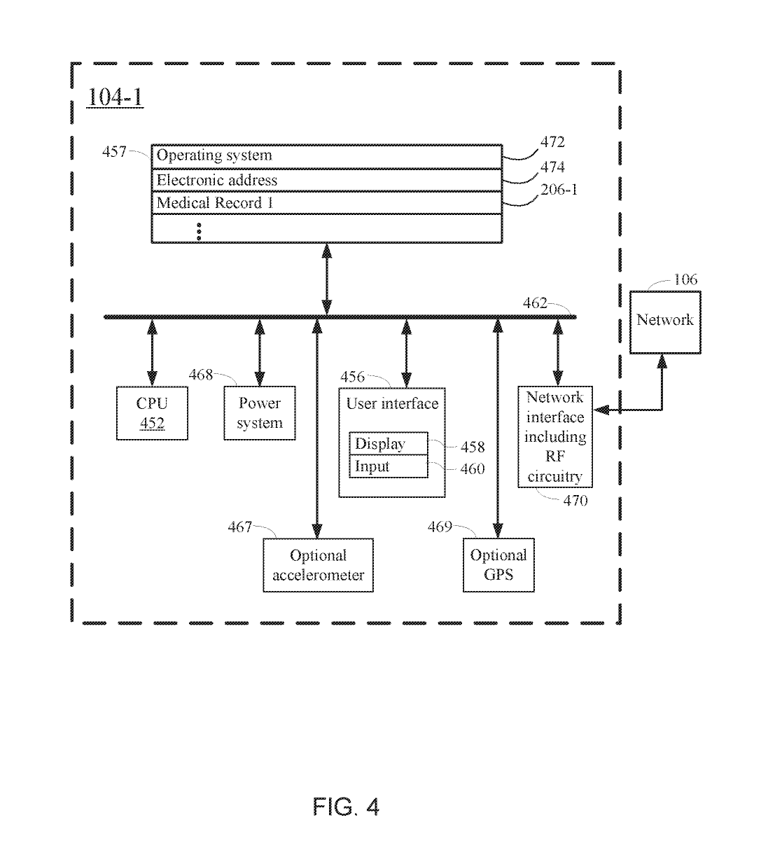

Moreover, health care providers, or health systems, delegate regular monitoring of subject data received by the disclosed systems and methods to personnel who monitor subjects closely and alert health care providers as needed. In some embodiments, referring to FIG. 4, a medical practitioner device 104 associated with a medical practitioner, is a desktop computer. In other embodiments, the medical practitioner device 104 is not a desktop computer but rather is a tablet computer, smart phone, emergency vehicle computer, or other form or wired or wireless networked device. In some embodiments, the medical practitioner device 104 has any or all of the circuitry, hardware components, and software components found in the parsing device 200 depicted in FIG. 3. In the interest of brevity and clarity, only a few of the possible components of the medical practitioner device 104 are shown in order to better emphasize the additional software modules that are installed on device 104.

In typical embodiments, the medical practitioner device 104 has one or more processing units (CPUs) 452, a network or other communications interface 470, a memory 457 (e.g., random access memory), a user interface 456, the user interface 456 including a display 458 and input 460 (e.g., keyboard, keypad, touch screen), an optional accelerometer 467, an optional GPS 469, one or more communication busses 462 for interconnecting the aforementioned components, and a power system 468 for powering the aforementioned components. In some embodiments, the input 460 is touch-sensitive display, such as a touch-sensitive surface. In some embodiments, the user interface 456 includes one or more soft keyboard embodiments. In further embodiments, the soft keyboard embodiments include standard (QWERTY) and/or non-standard configurations of symbols on the displayed icons.

Medical practitioner device 104 optionally includes, in addition to accelerometer(s) 467, a magnetometer (not shown) and a GPS 469 (or GLONASS or other global navigation system) receiver for obtaining information concerning the device 104.

It should be appreciated that the medical practitioner device 104 is only one example of a multifunction device, and that the medical practitioner device 104 optionally has more or fewer components than shown in FIG. 4 (or in FIG. 3), optionally combines two or more components, or optionally has a different configuration or arrangement of the components. The various components shown in FIG. 4 are implemented in hardware, software, firmware, or a combination thereof, including one or more signal processing and/or application specific integrated circuits. Memory 457 of the medical practitioner device 104 illustrated in FIG. 4 optionally includes high-speed random access memory and optionally also includes nonvolatile memory, such as one or more magnetic disk storage devices, flash memory devices, or other non-volatile solid-state memory devices. Access to memory 457 by other components of the medical practitioner device 104, such as CPU(s) 452.

In some embodiments, the memory 457 of the medical practitioner device stores:
an operating system 472 that includes procedures for handling various basic system services;
an electronic address 474 (e.g., a mobile phone number, social media account, or e-mail address) that is used by the data parsing device 200 to provide information pertaining to the practice; and
various medical records 206 of the subjects of the medical practitioner.

Example Methods

Now that details of systems 200, 250 for determining whether a first human has an actionable risk for each of a plurality of hereditary cancers has been disclosed, details regarding the processes and features of the system, in accordance with various embodiments of the present disclosure, are disclosed with reference to FIGS. 5 through 9. In some embodiments, such processes and features of the system are carried out by the various modules described in example systems 200, 250, as illustrated in FIGS. 1 through 4.

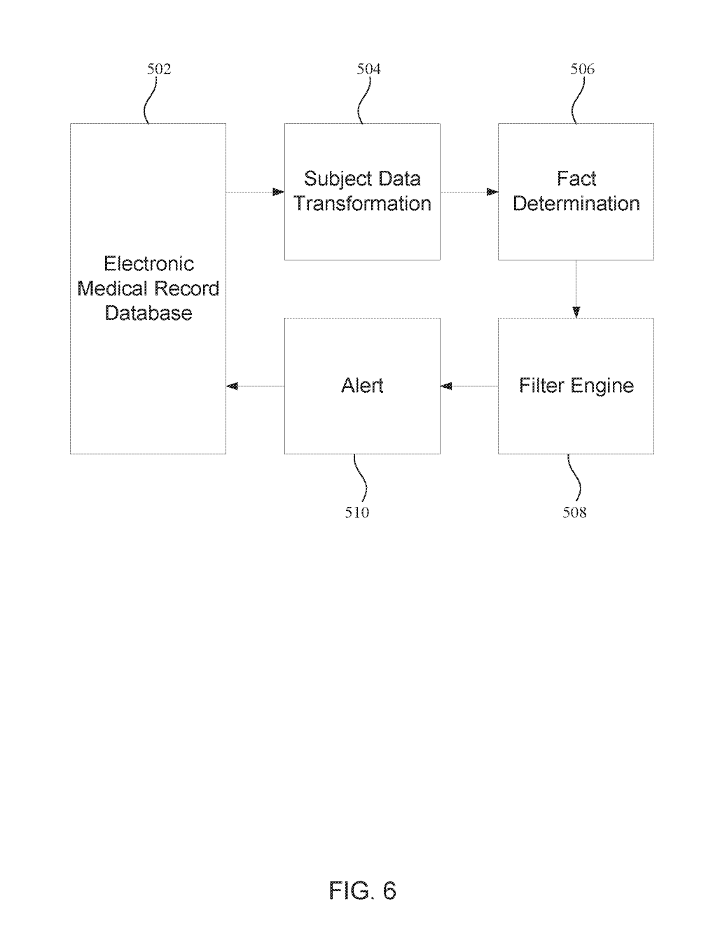

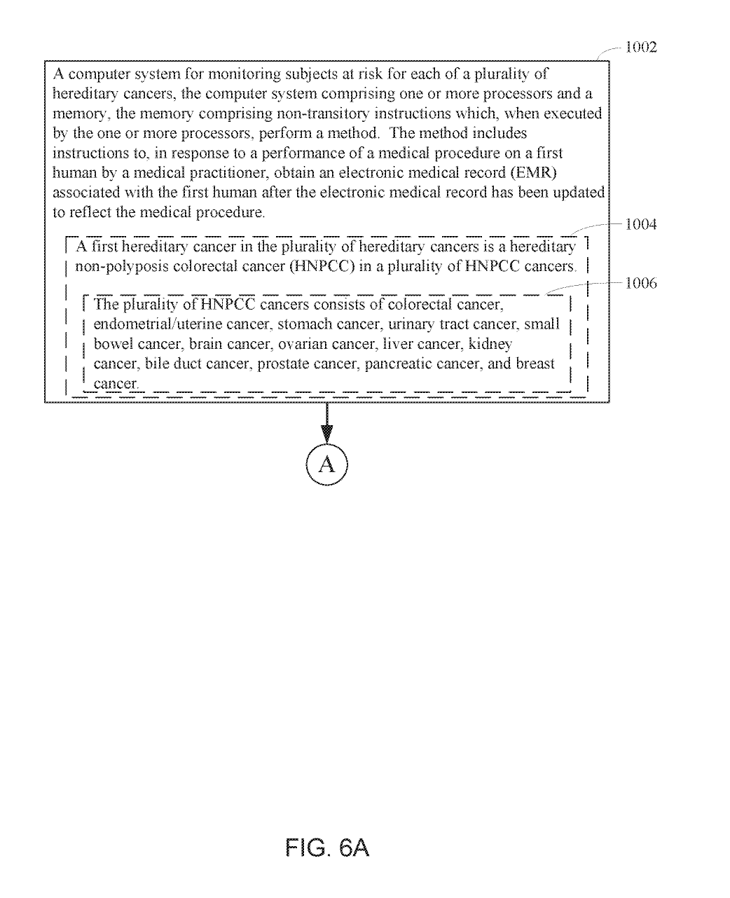

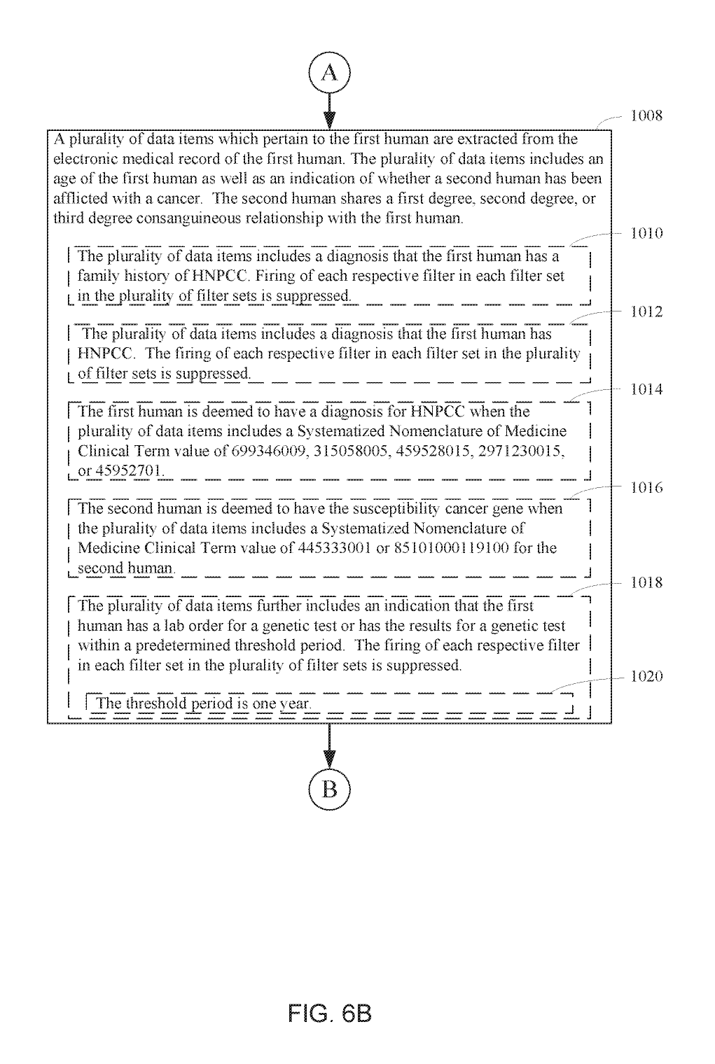

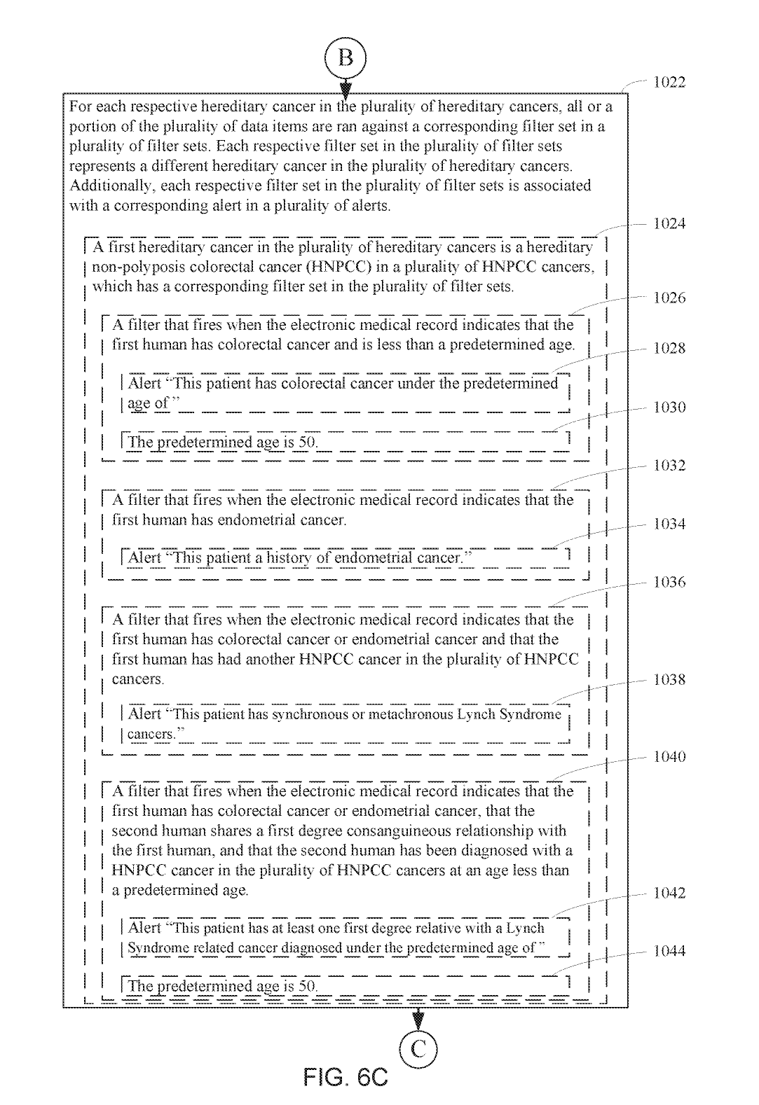

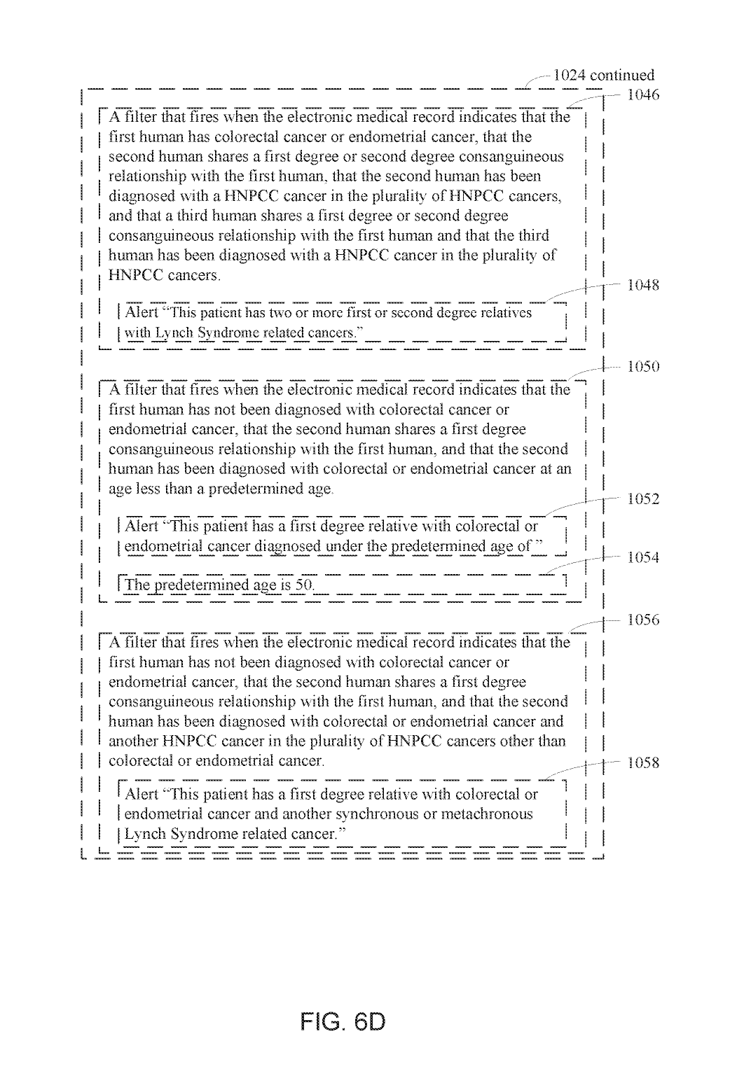

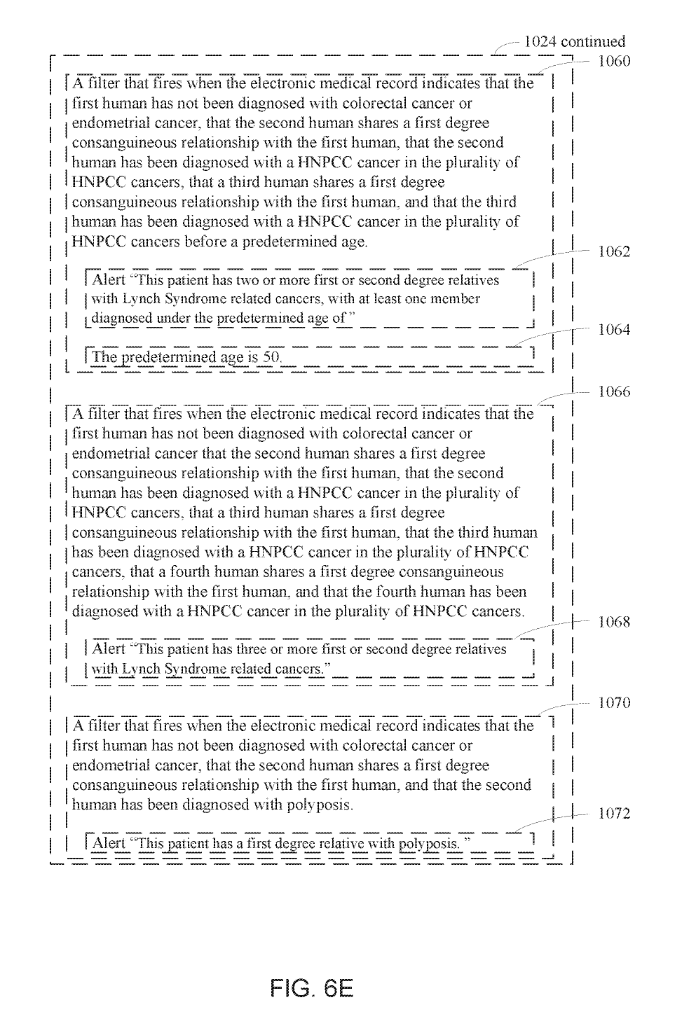

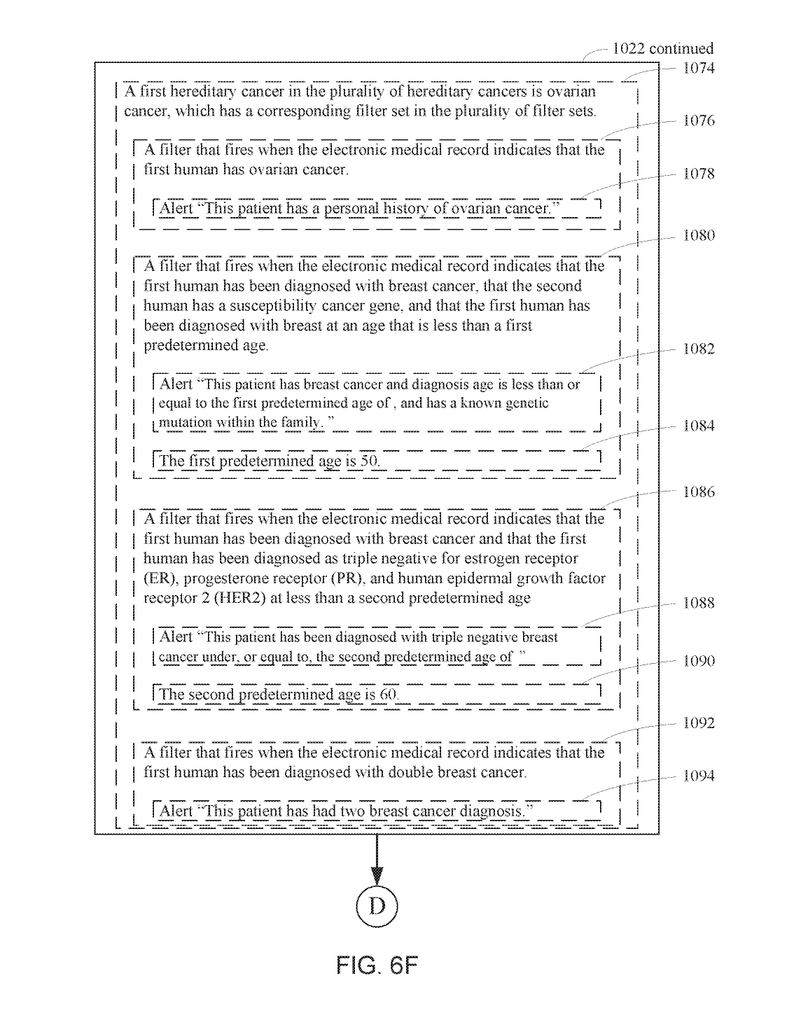

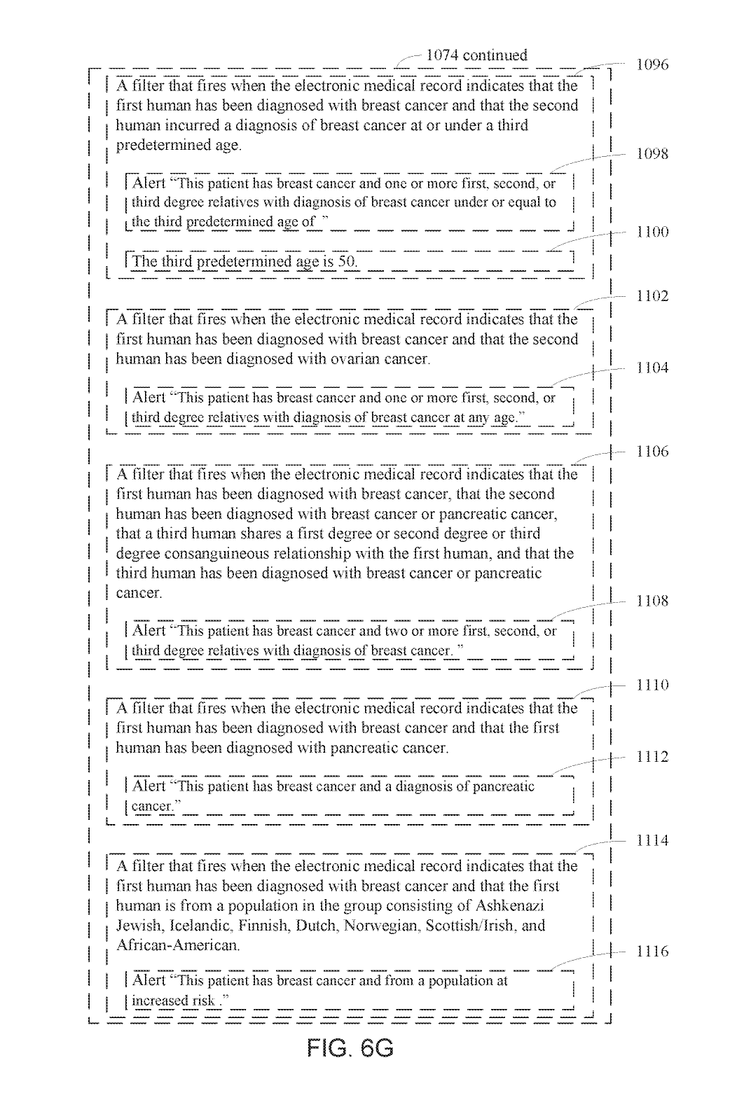

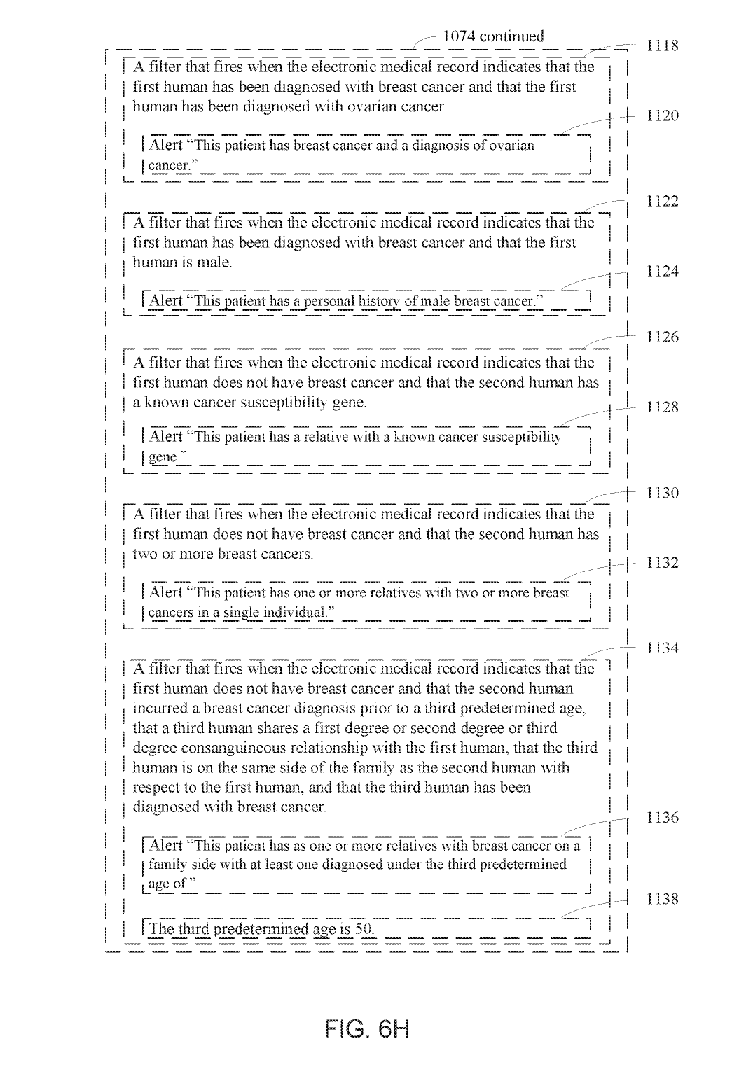

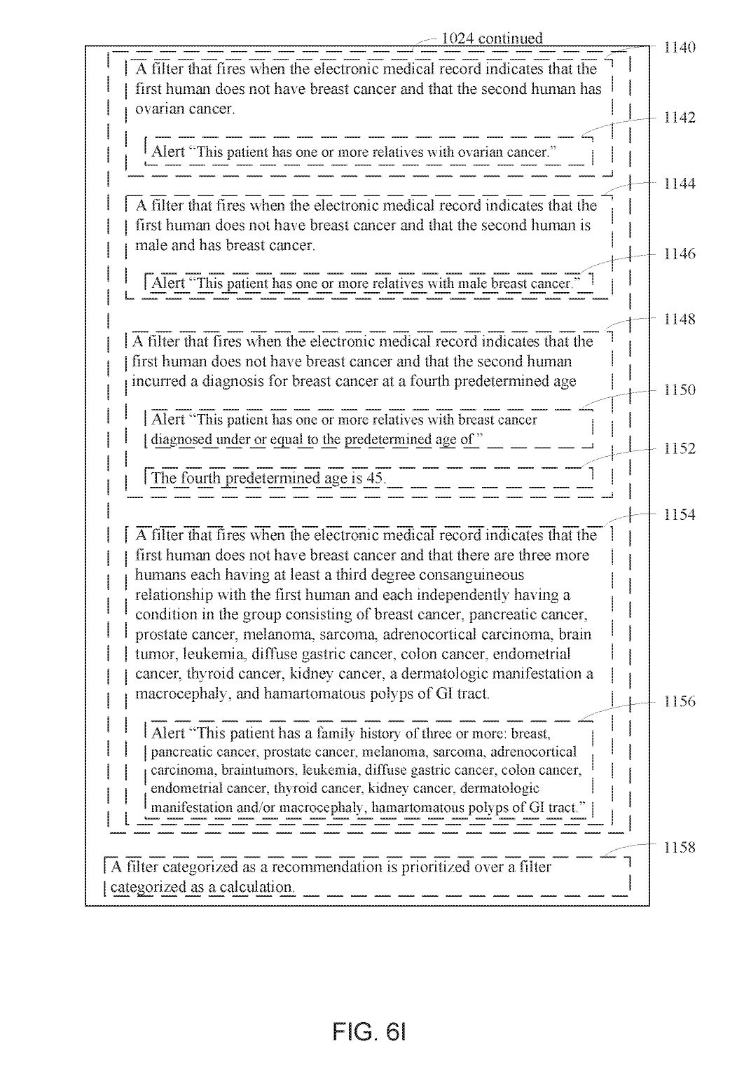

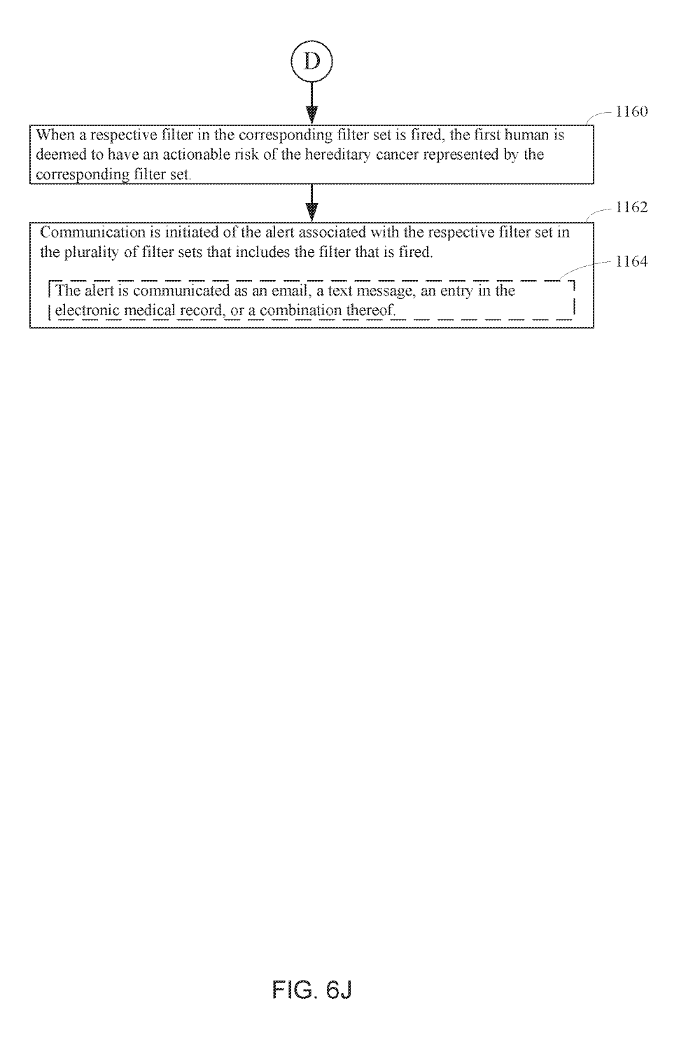

Referring to FIG. 5, a general workflow for monitoring a plurality of subjects for a plurality of hereditary diseases is described, e.g., in accordance with the methods 600, as illustrated in FIG. 6, and 900, as illustrated in FIG. 9. It should be understood that the present exemplary embodiment is intended for illustrative and descriptive purposes and is not intended to limit the spirit or scope of the present disclosure.

As described in FIG. 5, an electronic medical record database 204 includes a plurality of medical records. Each medical record 206 in the plurality of medical records is associated with a corresponding subject (502). A medical record is determined to be of interest, and an application programming interface (API) communication is opened with the EMR record database to extrapolate and parse subject data of the medical record into a desired format (504). From the translated subject data, at least one fact or condition is discovered (506). The at least one fact or condition is run through the filter data store 216 (e.g., filter engine) comprising a plurality of predetermined filters (508). When the at least one fact or condition triggers a filter in the plurality of filters, an alert is communicated to, e.g., a medical practitioner, and associated with the corresponding medical record (510). When the at least one fact or condition does not trigger a filter, the filter engine is temporarily suppressed, e.g., and an alert is not communicated. In some embodiments, when no filters are triggered by the medical information, a transmission is communicated, e.g., to a medical practitioner, indicating that no filter was triggered by the medical information.

Further details about processes and features of the systems of FIG. 1-4, in accordance with some embodiments of the present disclosure, are disclosed with reference to FIGS. 6A through 6J. As such, FIG. 6 illustrates methods for monitoring subjects for hereditary cancers, in accordance with some embodiments of the present disclosure.

FIG. 6 is a flow diagram illustrating a method 600 for determining whether a first human has an actionable risk for each of a plurality of hereditary cancers. Some or all portions of method 600 are performed at a computer system (e.g., computer system 200 or 250 in FIGS. 2 and 3) having one or more processors, and memory storing one or more programs for execution by the one or more processors for determining whether a first human has an actionable risk for each of a plurality of hereditary cancers. Some operations in method 600 are, optionally, combined and/or the order of some operations is, optionally, changed.

Block 1002.

With reference to block 1002 of FIG. 6A, the goal of one aspect of the present disclosure is to monitor subjects for hereditary cancers (e.g., by determining whether the subject has, or is likely to have, a hereditary risk for one or more types of cancers) using a computer system (e.g., device 200 of FIG. 3 and/or device 104 of FIG. 4).

In some embodiments, the methods disclosed herein rely on personal and/or familial medical information about a patient. In some embodiments, this familial medical history is obtained by asking the subject a series of questions, e.g., related to their personal medical history and/or family medical history. In other embodiments, familial medical information is obtained directly from a medical record associated with a family member of the subject, rather than directly from the subject. In some embodiments, the medical information is obtained prior to a medical consultation. In some embodiments, the medical information is obtained at a medical consultation. In some embodiments, the personal and familial medical information is obtained from a prior medical consultation. Example questions include, but are not limited, to:
Have you had breast cancer? If yes, what age?
Have you had colon cancer? If yes, what age?
Have you had endometrial cancer? If yes, what age?
Have you had ovarian cancer? If yes, what?
Has a member of your family had breast cancer? If yes, what age?
Has a member of your family had colon cancer? If yes, what age?
Has a member of your family had endometrial cancer? If yes, what age?
Has a member of your family had ovarian cancer? If yes, what?
   In some instances, a subset of these questions are asked. In some embodiments, more questions are asked. In some instance some of the above questions in addition to other questions are asked.

The medical practitioner then updates an EMR of the first subject (e.g., personal record 208 of FIG. 2) at a local device (e.g., medical practitioner device 104 of FIGS. 1 and 4) with information (e.g., data items 210) derived from the provided series of questions.

In some embodiments, instructions are sent to obtain the electronic medical record (EMR) associated with the first subject after the EMR has been updated, or created when the subject is a new client, to reflect the medical procedure and questions. In some embodiments, a request for medical information is sent prior to a medical consultation (e.g., and not in response to and/or immediately after updating the subject's medical information). In some embodiments, a request for medical information is sent following a medical consultation (e.g., and not in response to and/or immediately after updating the subject's medical information).

In some embodiments, the instructions are sent via an application programming interface (API) call to the EMR system (e.g., EMR system 250 of FIG. 2). However, the present disclosure is not limited thereto. For example, in some embodiments the instructions are sent directly to the EMR system 250.

Blocks 1004-1006.

Referring to blocks 1004-1006 of FIG. 6A, in some embodiments, a first hereditary cancer is a hereditary non-polyposis colorectal cancer (HNPCC) related cancer, e.g., from among an enumerated list of HNPCC cancers. In some embodiments, the enumerated list of HNPCC cancers includes any two or more, any three or more, any four or more, or any five or more cancers selected from among colorectal cancer, endometrial/uterine cancer, stomach cancer, urinary tract cancer, small bowel cancer, brain cancer, ovarian cancer, liver cancer, kidney cancer, bile duct cancer, prostate cancer, pancreatic cancer, and breast cancer. In some embodiments, the enumerated list of HNPCC cancers includes colorectal cancer, endometrial/uterine cancer, stomach cancer, urinary tract cancer, small bowel cancer, brain cancer, ovarian cancer, liver cancer, kidney cancer, bile duct cancer, prostate cancer, pancreatic cancer, and breast cancer. Moreover, the present disclosure is not limited to the present listing of HNPCC cancers, as continual research and development allows support of the present disclosure in relation to other additional diseases. For instance, when links between data items 210 of the subject and family members are discovered to relate to a new hereditary disease, this new hereditary disease is implemented with the systems and methods of the present disclosure.

Block 1008.

Referring to block 1008 of FIG. 6B, in the method data items 210 that pertain to the subject are extracted (e.g., retrieved) from the electronic medical record of the subject. In some embodiments, the data items (e.g., data 210 of FIG. 2) include at least an age of the subject, answers to the previously described series of questions, as well as an indication of whether another human has been afflicted with a cancer. For instance, in some embodiments, the data items include a cancer diagnosis status (or an indication that that the subject has not been previously diagnosed with cancer, e.g., by the lack of information on a previous cancer diagnosis) and a familial history of hereditary cancer (or an indication that there is no familial history of cancer, e.g., by the lack of information on a hereditary cancer diagnosis for a family member). However, the present disclosure is not limited thereto as the contents of the data items 210 are not constrained to specific types of information. In some embodiments, the other human shares a first degree, second degree, or third degree consanguineous relationship with the subject, and thus is referred to herein as a family member 212 of the subject 208.

In some embodiments, e.g., after data items 210 have been input to the EMRs of the subjects (e.g., personal records 208), upcoming appointments, e.g., for the next day, are determined and EMRs 206 of the subjects corresponding to the upcoming appointments, e.g., for the next day, are parsed prior to the appointments. In some embodiments, the data retrieved from the medical records are applied to an algorithm (e.g., as described herein with reference to FIGS. 5 and/or 9) that evaluates whether a subject is at an elevated risk for a hereditary cancer (e.g., has a risk that is greater than the average population risk), and communicates a recommendation as to whether the subject should undergo genetic screening for one or more hereditary cancer risk factors is provided to a medical practitioner. In this fashion, the medical practitioner can recommend genetic screening to the subject at their upcoming medical consultation, e.g., as opposed to in a follow-up communication. Advantageously, this expedites the screening process, as the subject is able to discuss with benefits and risks of genetic screening with the medical practitioner during the consultation and/or provide a biological sample (e.g., a saliva sample) at their consultation. In some embodiments, e.g., at the end of each day or appointment, the data parsing device 200 scans the medical record data store 204 and/or individual medical records 206 to determine any further suggestions, recommendations, and/or alerts 222 to communicate to the medical practitioner, the medical practitioner device 104, and/or the subject.

In some embodiments, a data parsing device (e.g., data parsing device 200 of FIG. 3) retrieves the data items (e.g., data item 210 of FIG. 2) through various API calls to an electronic medical records system (e.g., EMR system 250 of FIG. 2). The API calls are through an API connection. In some embodiments, API connection is communicated with an Athena health platforms. In some embodiments, the API connection(s) is authenticated using basic authentication, including, but not limited to, key, secret, and version specified authentication. When a GET, a POST, or similar HTTP request method is called, the argument is converted into the proper form for a request, and the subsequent result is decoded from JavaScript Object Notation (JSON) and returned as either a JSON object or a JSON array.

In some embodiments, in order to get a patient list via a new appointment schedule before the schedule occurs, the following commands are input:
GET /v1/{practiceid}/appointments/booked
GET /patientID

In some embodiments, in order to retrieve data items 210, which include demographics, social history, family history, problems list, past medical history, lab results, lab orders, encounters, diagnosis charted during an encounter, and the like from each patients EMR 206, the following commands are input:
GET /v1/{practiceid}/patients
GET /chart/{patientid}/socialhistory
GET /v1/{practiceid}/chart/{patientid}/familyhistory
GET /v1/{practiceid}/chart/{patientid}/problems
GET /chart/configuration/medicalhistory
GET /v1/{practiceid}/chart/{patientid}/analytes
GET /v1/{practiceid}/reference/order/lab
GET /chart/patientid/encounters
GET /chart/encounter/encounterid/diagnosis

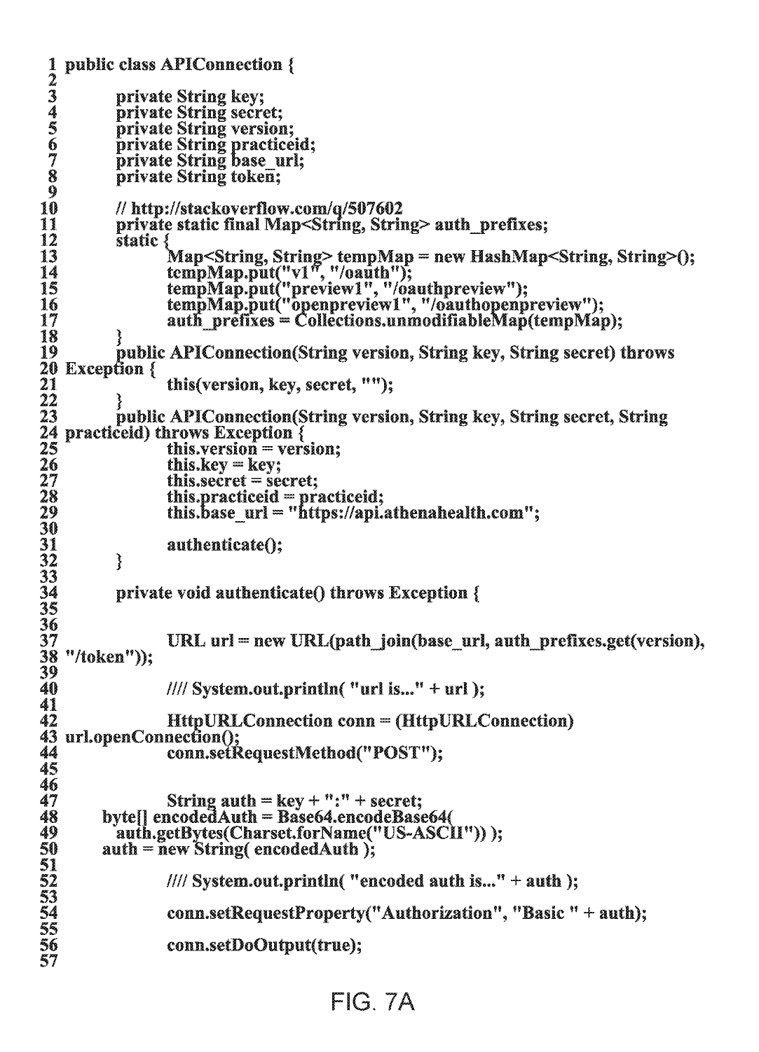

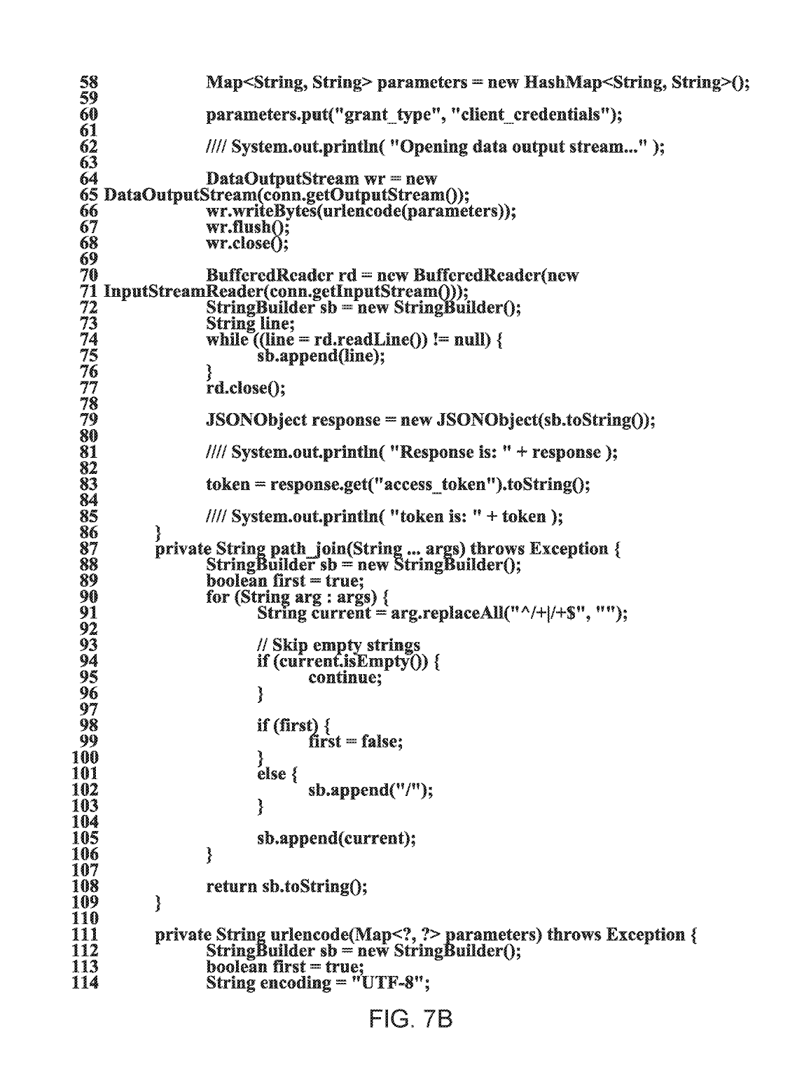

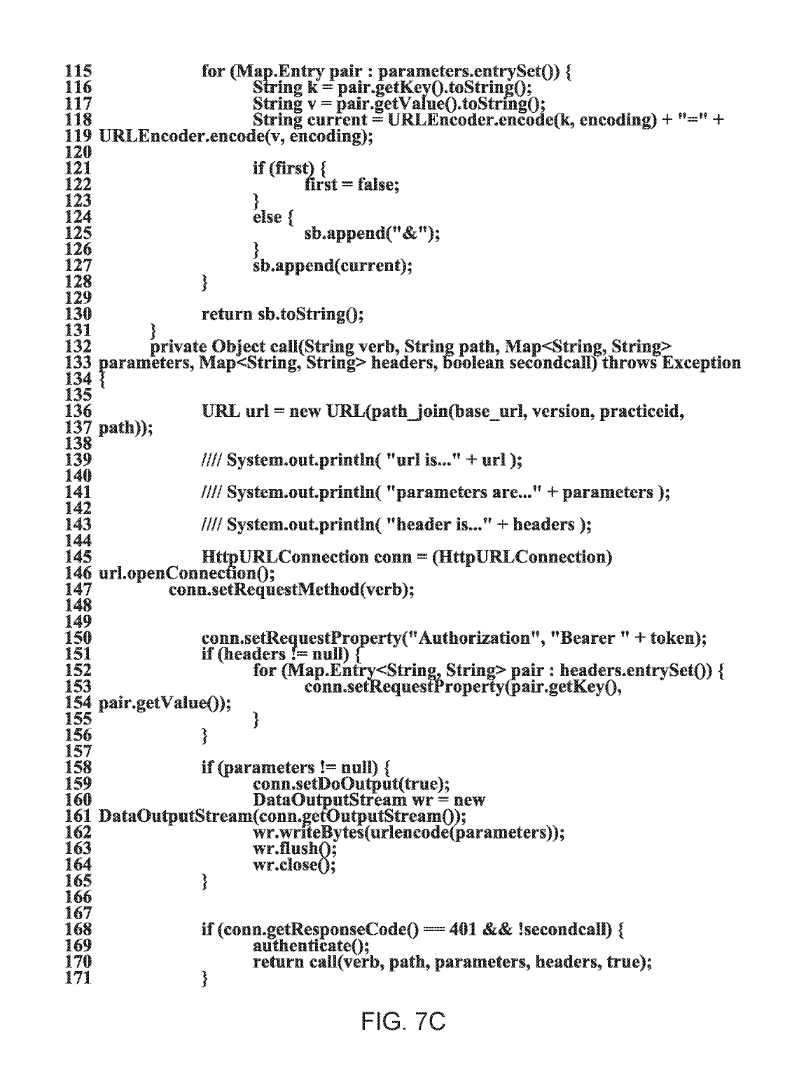

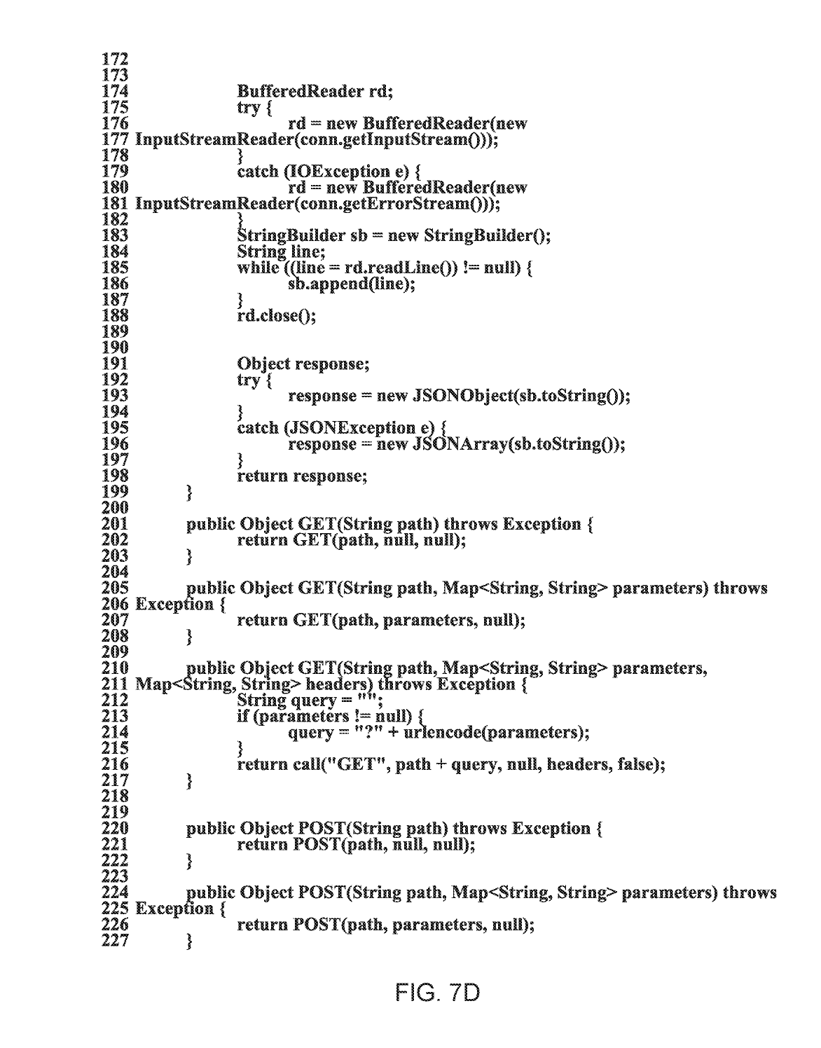

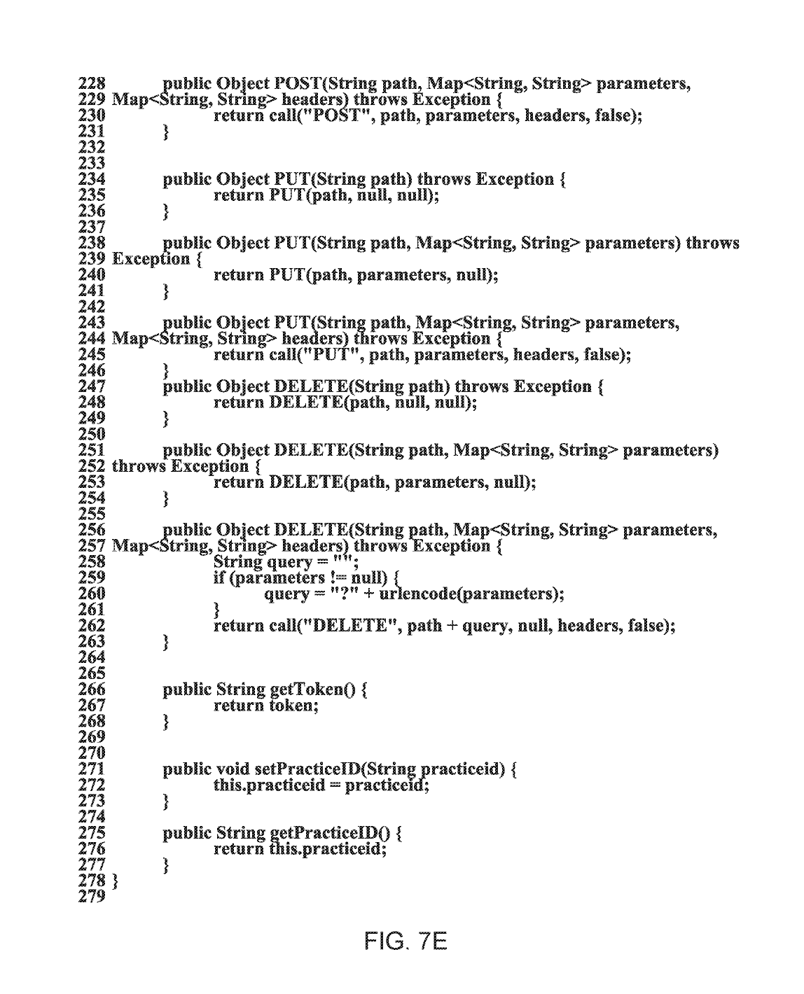

Briefly referring to FIG. 7, JAVA code describing the extraction of the data items 210 from the EMR system 250 will be described. Referring to FIGS. 7A and 7B, lines 1 through 86 are configured to parse the hypertext transfer protocol (HTTP) connection and basic authentication from the API calls. When an object is constructed, authentication is attempted using a key, secret, and specified version. Access tokens are stored for later use. Referring to FIGS. 7B and 7C, lines 87 through 131 are configured to join a variety of arguments into a valid logic path. Parameters, which are keys, values, and data items 210, are converted into a URL query string. Referring to FIGS. 7C and 7D, lines 132 through 199 are configured to perform the API call. Specifically, lines 158 through 165 illustrate code to set the request parameters. When the API call returns a failure or error, the call is repeated once more, as illustrated by lines 168 through 171. Depending on the result of the call, the API response of the call is determined to be in an input stream on success and in an error stream on failure, as illustrated by lines 174 through 188 of FIG. 7D. Furthermore, referring to FIGS. 7D and 7E, lines 201 through 263 illustrate a variety of requests including DELETE, GET, POST, and PUT which are utilized to extract the data items 210.

Blocks 1010-1020.

In some embodiments, the data items 210 include a diagnosis that the subject has a family history of HNPCC. In such embodiments, the data items 210 includes a diagnosis that the subject has HNPCC. In some embodiments, the subject is deemed to have a diagnosis for HNPCC when a data item 210 in the personal record 208 of the subject includes a Systematized Nomenclature of Medicine Clinical Term value of 699346009, 315058005, 459528015, 2971230015, or 45952701. In some embodiments, the family member is deemed to have a susceptibility cancer gene when the data items 210 of the family member includes a Systematized Nomenclature of Medicine Clinical Term value of 445333001 or 85101000119100. In some embodiments, as described in detail herein, when the family member is deemed to have a hereditary cancer susceptibility gene (e.g., an allele associated with a hereditary cancer), there is an increased risk that the subject will have the corresponding cancer in their lifetime. For instance, in some embodiments, when mutations in the BRCA1 and BRCA2 genes are discovered in a family member of the subject, there is an increased susceptibility for the subject to have breast cancer, ovarian cancer, and other various cancers (Brody et al., 1998, “Breast Cancer Susceptibility Genes,” BRCA1 and BRCA2,” Medicine 77, p. 208).

In some embodiments, the data items 210 include a race of the subject, a smoking status of the subject, an allergy status of the subject, a diabetes status of the subject, a blood pressure of the subject, a cholesterol level of the subject, an organ problem history of the subject, a metabolite level of the subject, an electrolyte level of the subject, a white blood cell count of the subject, or a bone marrow cell count of the subject.

In some embodiments, the data items 210 further include an indication that the subject has a lab order for a genetic test or has the results for a genetic test within a predetermined threshold period. For example, in some embodiments the data items 210 indicate the results and/or scheduling of a complete blood count test, the results and/or scheduling of a blood protein test, the results and/or scheduling of a tumor marker test, the results and/or scheduling of a circulating tumor cell test, the results and/or scheduling of a urinalysis test, the results and/or scheduling of a cancer gene mutation test, the results and/or scheduling of a cytogenetic analysis, and/or the results and/or scheduling of an immunophenotyping. However, the present disclosure is not limited thereto as other lab tests and results are included in accordance with a design of the present disclosure. In such embodiments, the predetermined threshold period is six months. In some embodiments, the predetermined threshold period is one year. In some embodiments, the predetermined threshold period is two years, three years, four years, or five years. In some embodiments, the predetermined threshold period is ten years, twenty years, or fifty years.

As previously described, an aspect of the present disclosure relates to the ability to extract and parse information from an EMR 206 into a standard format for analysis. For instance, in some embodiments information of a data item 210 in the EMR 206 is in the form of a slide bar, an input field, a drop down menu, a variety of radio buttons (e.g., option buttons), a free-text form, a structured-text form, a picture, a video, or the like. Likewise, not only does the precise form of data entry vary from field to field and EMR to EMR, but also from clinician to clinician. Thus, as a process of extracting the data, the data much be translated into a standard format for analysis. Such a process will be described in greater detail infra.

Block 1022.

Referring to block 1022 of FIG. 6C, in the method, for each respective hereditary cancer, all or a portion of the data items 210 are run against a corresponding filter set (e.g., filter set 218-1 of FIG. 3) in a plurality of filter sets (e.g., filter sets 218 of FIG. 3). Each respective filter set in the plurality of filter sets represents a different hereditary cancer. In some embodiments, at least one filter set in the plurality of filter sets represents at least two hereditary cancers (e.g., filter set 218-3 is associated with breast cancer and colon cancer). Likewise, in some embodiments, at least one filter set in the plurality of filter sets represents no hereditary cancers (e.g., filter set 218-4 is associated with a lack of hereditary cancers in a subject). In many embodiments, the filters 220 are categorized as a recommendation or a calculation. As used herein, a filter categorized as a recommendation, when fired, will suggest or promote a cause to action by the subject or medical practitioner. For instance, in one implementation a recommendation causes communication of “This patient should be genetically screened for a disease.” Likewise, as used herein, a filter categorized as a calculation, when fired, will recite a fact regarding the subject or family history of the subject. For instance, in another implementation a calculation causes communication of “This patient has two or more second or third degree family members with cancer susceptibility genes.” In some embodiments, the filters 220 consider indications of a familial cancer syndrome, including but not limited to identification of a variety of cases of a same or similar type of cancer, cancers occurring at younger ages than typically expected in a population, at least two types of cancer in an individual, cancers occurring in both pairs of an organ (e.g., ocular melanoma in both the left eye and the right eye), cancers occurring in multiple generations (e.g., son diagnosed with colon cancer, father diagnosed with colon cancer, and grandfather diagnosed with colon cancer). In some embodiments, each respective filter set 218 in the plurality of filter sets is associated with at least one corresponding alert 222. In some embodiments, the alert 222 is communicated by an email to the subject, the medical practitioner device 104, and/or the medical practitioner, a text message to the subject, the medical practitioner device 104, and/or the medical practitioner, a computer generated audio message to the subject, the medical practitioner device 104, and/or the medical practitioner, an input in the EMR 206 of the subject (e.g., data item 210 of FIG. 2), or a similar form of communication to the subject, the medical practitioner device 104, and/or the medical practitioner. In some embodiments, an alert 222 is communicated indicating a consult note for any subjects that need screening following implementation of the systems and methods of the present disclosure. In some embodiments, a practitioner or provider includes a referral note in the alert 222. In typical embodiments, the referral note supplements a consult note. In some embodiments, in order to send results to a provider or practitioner, the following command is input:
POST /v1/{practiced}/patients/{patientid}/documents

In some embodiments, firing of a respective filter 220 in each filter set 218-1 in the plurality of filter sets is suppressed when at least one of the above data items 210 are parsed, e.g., when the algorithm identifies an indication that the subject has already been diagnosed as having a hereditary cancer risk and/or is already scheduled to undergo further cancer screening, e.g., genetic or otherwise. In some embodiments, the data items 210 include various physiological measurements. In some embodiments, the data items 210 includes insurance information of the subject, lab test information of the subject include test results, prescription medication information of the subject including current and past prescriptions, mental health information of the subject, a history of emergency room visits of the subject, a history of hospitalizations of the subject, and general biometric information of the subject (e.g., height of the subject, eye color of the subject, hair color of the subject).

In some embodiments, the data items 210 includes data items from the EMR of family members (e.g., family member record 212 of FIG. 2) and/or information pertaining to a family member in an EMR 206 of a subject (e.g., data item 210 of FIG. 2), e.g., information that tends to indicate whether the family member has been previously been diagnosed with a hereditary cancer, such as insurance information of the family of the subject, lab test information of the family of the subject, prescription medication information of the family of the subject, mental health information of the family of the subject, and general biometric information of the family of the subject.

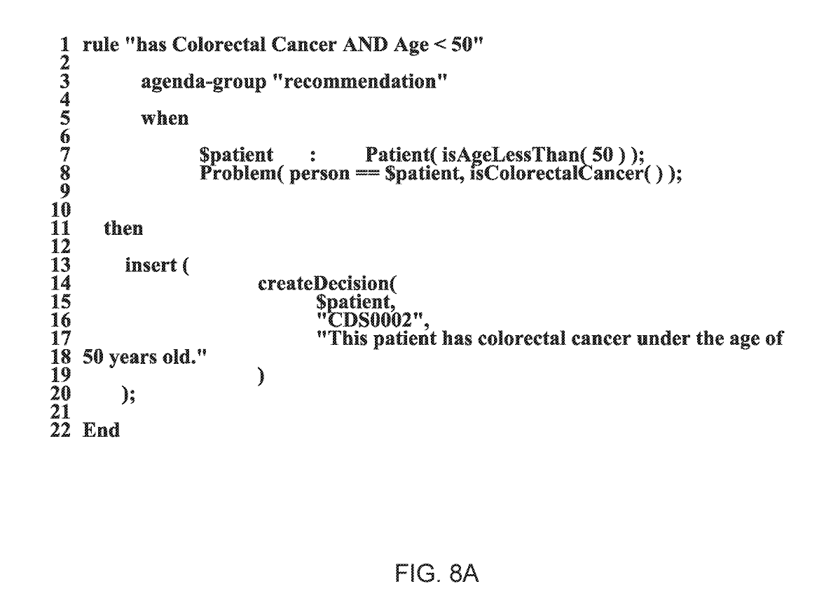

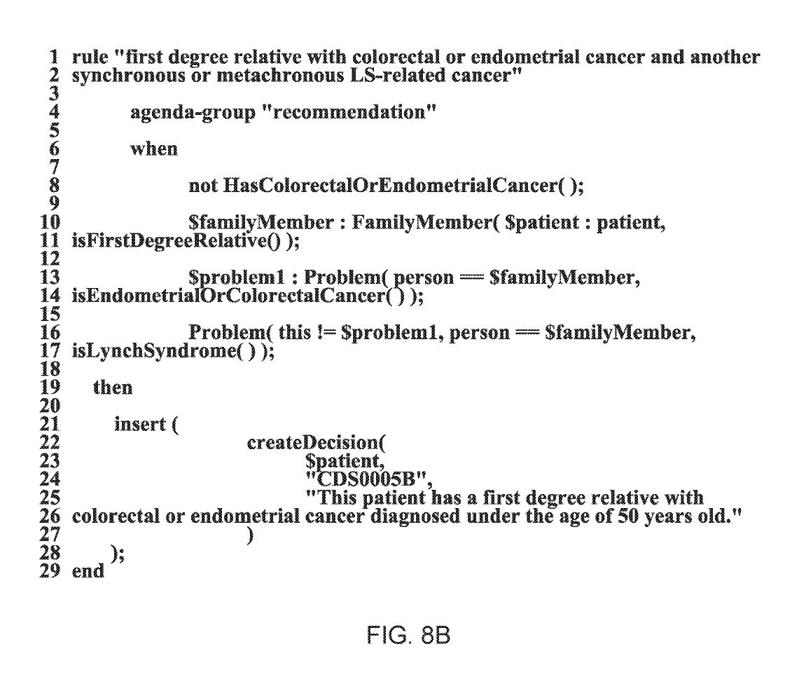

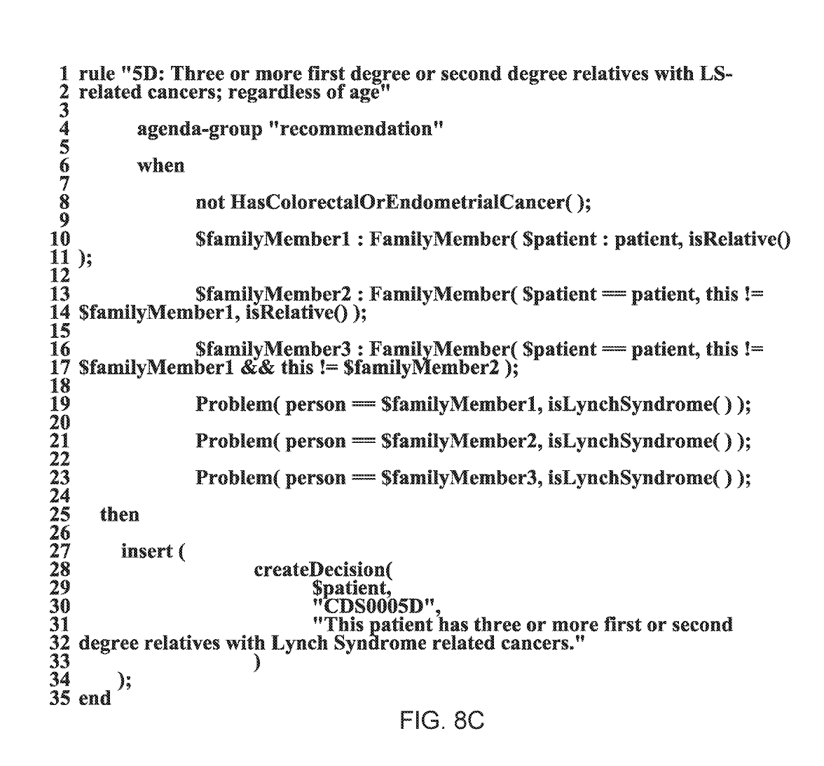

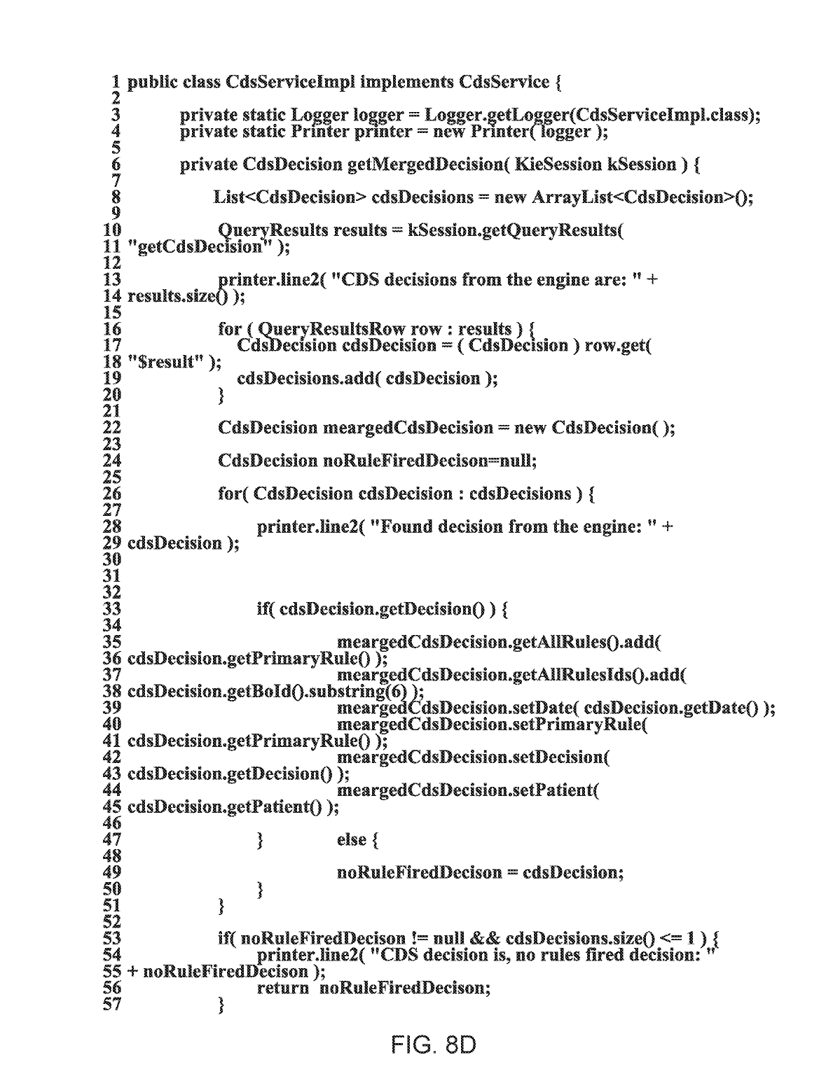

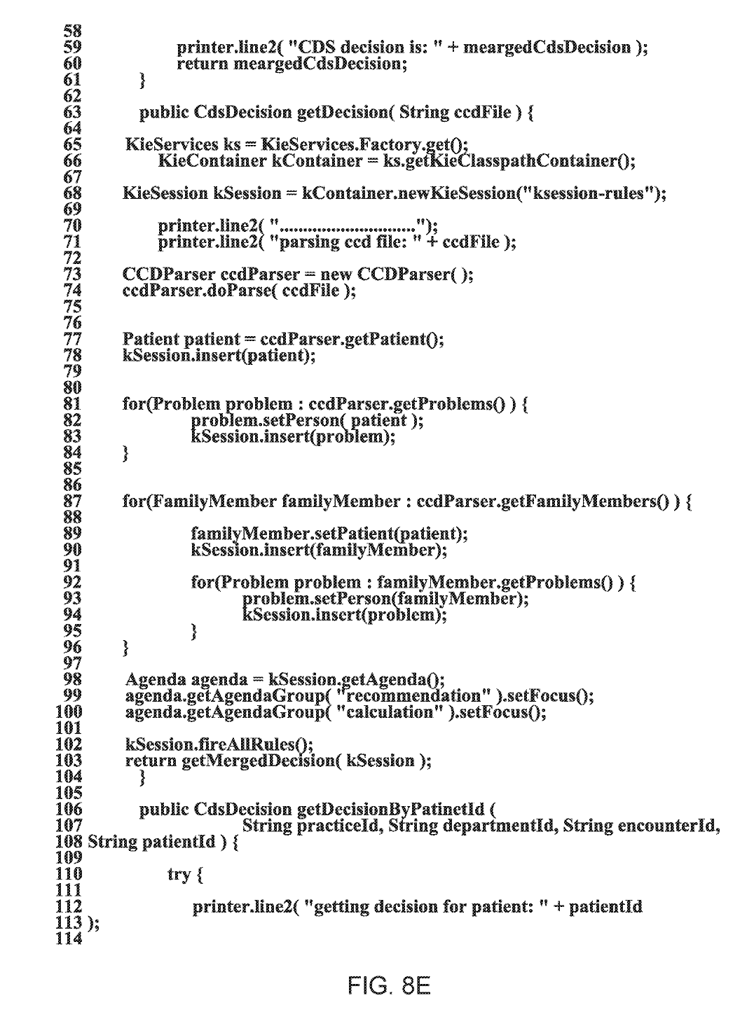

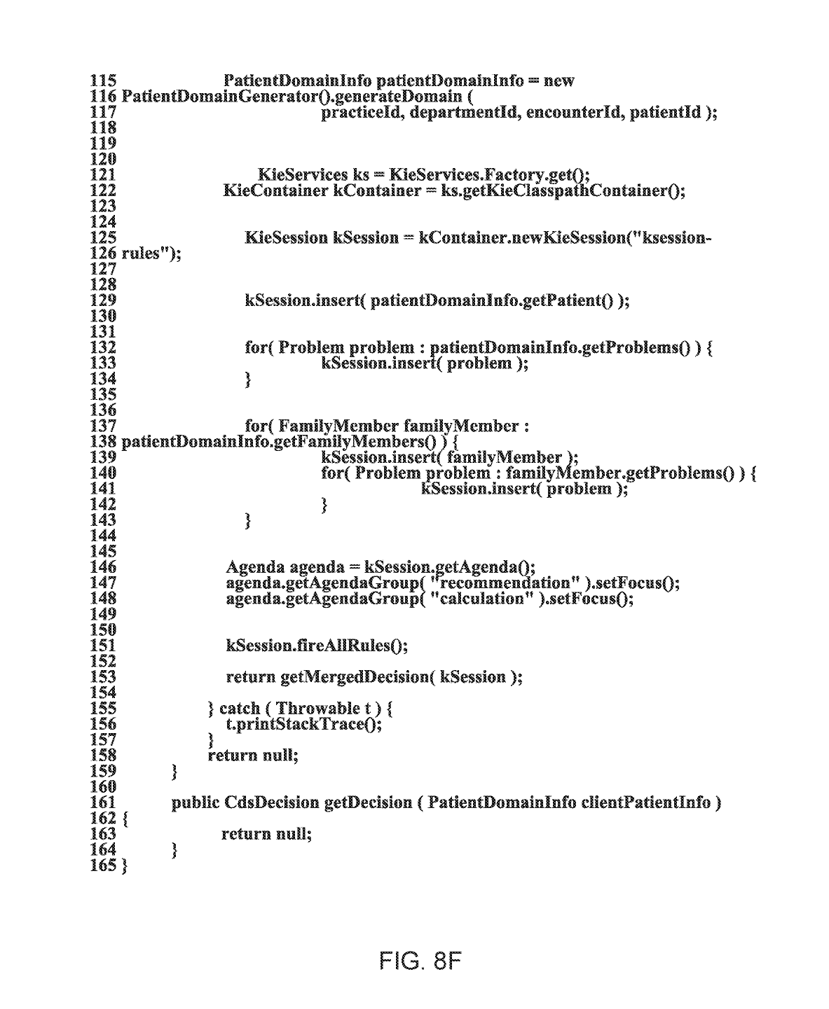

As previously described, in some embodiments, various filters 220 are associated with alerts 222 identifying actionable risks related to the health of a subject (e.g., an increased risk for one or more hereditary cancers). In some embodiments, filters 220 are designed with Boolean operators (e.g., IF condition W AND {condition X OR condition Y) THEN condition Z). Referring to FIG. 8, JAVA code is shown for an example implementation of an algorithm for identifying an increased risk of a hereditary cancer risk. As shown in FIG. 8, Boolean operators are used in this example. However, the present disclosure is not limited thereto. Furthermore, in the example implementation presented, only a single subject is in a knowledge base for clarity and simplicity. However, the present disclosure is not limited thereto. For example, in some embodiments hundreds of thousands of subjects, or more, are in a knowledge base.

In the example implementation described below, varieties of exemplary alerts 220 are presented. However, the foregoing alerts are in no way limiting of the present disclosure and are intended to explain the principles of the present disclosure.

Block 1024.

In some embodiments, a first hereditary cancer is a hereditary non-polyposis colorectal cancer (HNPCC) in an enumerated list of HNPCC cancers (e.g., Lynch Syndrome related cancers). Examples of Lynch Syndrome related cancers include colorectal cancer, endometrial/uterine cancer, stomach cancer, urinary tract cancer, small bowel cancer, brain cancer, ovarian cancer, liver cancer, kidney cancer, bile duct cancer, prostate cancer, pancreatic cancer, and breast cancer. In some embodiments, the first hereditary cancer (e.g., an HNPCC related cancer) has a corresponding filter set in the plurality of filter sets (e.g., filter set 218-1 of filter sets 218 of FIG. 3).

Blocks 1026-1030.

In some embodiments, a filter 220 fires when the electronic medical record 206 (e.g., information extracted from the medical record) of the subject indicates that the subject has colorectal cancer and is less than a predetermined age (e.g., the subject was diagnosed with colorectal cancer at a premature age). In some embodiments, the predetermined age is fifty years old, however, the skilled artisan will know how to appropriately select an age to serve the purpose of a particular implementation. E.g., in some embodiments, e.g., where more aggressive recommendations for genetic screening are desired, the predetermined age may be set at an older age, e.g., sixty years of age. In contrast, in some embodiments, e.g., where less aggressive recommendations for genetic screening are desired, the predetermined age may be set at a younger age, e.g., forty years of age. In some embodiments, when the filter 220 is fired, a corresponding alert 222 is communicated to the medical practitioner device 104, the medical record 206, and/or the subject. For example, “This patient has colorectal cancer under the age of 50,” and/or “It is recommended that this patient is genetically screened for a predisposition for HNPCC related cancers. In some embodiments, a filter 220 fires when the subject has colorectal cancer and is under (e.g., was diagnosed as a premature age) a predetermined age (e.g., fifty years old). Again, the skilled artisan will know how to select the predetermined age to meet the goals of a particular implementation. E.g., in some embodiments, the predetermined age is forty years old, while in other embodiments, the predetermined age is sixty years old. As shown in FIG. 8A, the above filter is described in Boolean operations as: IF subject HAS PROBLEM: colorectal cancer AND Age <50 THEN suggest testing. In some embodiments, this filter 220 is coded in JAVA, as shown in FIG. 8A.

Blocks 1032-1034.

In some embodiments, a filter 220 fires when the electronic medical record 206 indicates that the subject has endometrial cancer. In an example of such an embodiment, a corresponding alert 222 communicates to the medical practitioner device 104, the medical record 206, and/or the subject “This patient a history of endometrial cancer” to the subject and/or the medical practitioner.

Blocks 1036-1038.

In some embodiments, a filter 220 fires when the electronic medical record 206 indicates that the subject has colorectal cancer or endometrial cancer and another HNPCC cancer. In such embodiments, a corresponding alert 222 communicates to the medical practitioner device 104, the medical record 206, and/or the subject “This patient has synchronous or metachronous Lynch Syndrome cancers” to the subject and/or the medical practitioner.

Blocks 1040-1044.

In some embodiments, a filter 220 fires when the electronic medical record 206 indicates that the subject has colorectal cancer or endometrial cancer, the other human shares a first degree consanguineous relationship with the subject, and the other human has been diagnosed with a HNPCC cancer younger than a predetermined age (e.g., data item 210-3 indicates family member P is less than thirty-five and has a HNPCC cancer). In typical embodiments, the predetermined age is fifty years old. In some embodiments, the predetermined age is forty years old. In some embodiments, the predetermined age is sixty years old. In such embodiments, a corresponding alert 222 communicates to the medical practitioner device 104, the medical record 206, and/or the subject “This patient has at least one first degree relative with a Lynch Syndrome related cancer diagnosed under the age of 50” to the subject and/or the medical practitioner.

Blocks 1046-1048.

Referring to blocks 1046-1048 of FIG. 6D, in the method, in some embodiments, a filter 220 fires when the electronic medical record 206 indicates that the subject has colorectal cancer or endometrial cancer, the other human shares a first degree or second degree consanguineous relationship with the subject, the other human has been diagnosed with a HNPCC cancer in the plurality of HNPCC cancers, and an additional human has been diagnosed with a HNPCC cancer and shares a first degree or second degree consanguineous relationship with the subject. In such embodiments, a corresponding alert 222 communicates to the medical practitioner device 104, the medical record 206, and/or the subject “This patient has two or more first or second degree relatives with Lynch Syndrome related cancers.”

Blocks 1050-1054.

In some embodiments, a filter 220 fires when the electronic medical record 208 indicates that the subject has not been diagnosed with colorectal cancer or endometrial cancer (e.g., the filter is not associated with a hereditary cancer). In some embodiments, the filter 200 fires when the other human shares a first degree consanguineous relationship with the subject and has been diagnosed with colorectal or endometrial cancer younger than a predetermined age (e.g., equal to or less than forty-four years old). In some embodiments, the filter 220 fires when the subject has not been diagnosed with colorectal cancer or endometrial cancer and the other human has colorectal or endometrial cancer prior to a predetermined age (e.g., before the family member is fifty years old). In typical embodiments, the predetermined age is fifty years old. However, the present disclosure is not limited thereto. In some embodiments, the predetermined age is forty years old. In some embodiments, the predetermined age is sixty years old. In some embodiments, the predetermined age is twenty-one years old. Accordingly, a corresponding alert 222 communicates to the medical practitioner device 104, the medical record 206, and/or the subject “This patient has a first degree relative with colorectal or endometrial cancer diagnosed under the age of 50.” In other words, this filter is described by Boolean operations as: IF first degree relative with colorectal OR endometrial cancer AND Age of Diagnosis <50 THEN suggest testing. In some embodiments, this filter 220 is coded in JAVA, as shown in FIG. 8B.

Blocks 1056-1058.

In some embodiments, a filter 220 fires when the electronic medical record 206 indicates that the subject has not been diagnosed with colorectal cancer or endometrial cancer. The filter 220 also requires that the other human share a first degree consanguineous relationship with the subject and the other human has been diagnosed with colorectal or endometrial cancer as well as a different HNPCC cancer other than colorectal or endometrial cancer. In such embodiments, a corresponding alert 222 communicates to the medical practitioner device 104, the medical record 206, and/or the subject “This patient has a first degree relative with colorectal or endometrial cancer and another synchronous or metachronous Lynch Syndrome related cancer.”

Blocks 1060-1064.

Referring to blocks 1060-1064 of FIG. 6E, in the method, in some embodiments, a filter 220 fires when the electronic medical record 206 indicates that the subject has not been diagnosed with colorectal cancer or endometrial cancer. The filter 220 requires that the other human share a first degree consanguineous relationship with the subject, and has been diagnosed with a HNPCC cancer. Additionally, the filter 220 requires that an additional human shares a first degree consanguineous relationship with the subject, and has been diagnosed with a HNPCC cancer before a predetermined age. In typical embodiments, the predetermined age is fifty years old. However, the present disclosure is not limited thereto. In some embodiments, the predetermined age is thirty years old. In some embodiments, the predetermined age is forty years old. In some embodiments, the predetermined age is sixty years old. In such embodiments, a corresponding alert 222 communicates to the medical practitioner device 104, the medical record 206, and/or the subject “This patient has two or more first or second degree relatives with Lynch Syndrome related cancers, with at least one member diagnosed under the age of 50.”

Blocks 1066-1068.

In some embodiments, a filter 220 fires when the electronic medical record 206 indicates that the subject has not been diagnosed with colorectal cancer or endometrial cancer. The filter 220 also requires that the other human share a first degree consanguineous relationship with the subject, and has been diagnosed with a HNPCC cancer. Additionally, the filter 220 requires that an additional human share a first degree consanguineous relationship with the subject, and has been diagnosed with a HNPCC cancer in the plurality of HNPCC cancers. Furthermore, the filter 220 requires that a fourth human shares a first degree consanguineous relationship with the subject, and that the fourth human has been diagnosed with a HNPCC cancer in the plurality of HNPCC cancers. In such embodiments, a corresponding alert 222 communicates to the medical practitioner device 104, the medical record 206, and/or the subject “This patient has three or more first or second degree relatives with Lynch Syndrome related cancers.” In other words, two OR more first degree OR second degree relatives with Lynch Syndrome related cancers. An example code for the above is shown in FIG. 8C.

Blocks 1070-1072.

In some embodiments, a filter 220 fires when the electronic medical record 206 indicates that the subject has not been diagnosed with colorectal cancer or endometrial cancer, and that the other human shares a first degree consanguineous relationship with the subject and has been diagnosed with polyposis. In such embodiments, a corresponding alert 222 communicates to the medical practitioner device 104, the medical record 206, and/or the subject “This patient has a first degree relative with polyposis.”

Block 1074.

Referring to block 1074 of FIG. 6F, in the method, in some embodiments, a first hereditary cancer is ovarian cancer. When the hereditary cancer is ovarian cancer, a corresponding filter set (e.g., filter set 218-1 of FIG. 3) in the plurality of filter sets 218 is directed to ovarian cancer.

Blocks 1076-1078.

In some embodiments, a filter 220 fires when the electronic medical record 206 indicates that the subject has ovarian cancer. In such embodiments, a corresponding alert 224 communicates to the medical practitioner device 104, the medical record 206, and/or the subject “This patient has a personal history of ovarian cancer.”

Blocks 1080-1084.

In some embodiments, a filter 220 fires when the electronic medical record 206 indicates that the subject has been diagnosed with breast cancer. The filter 220 further requires that the other human has a susceptibility cancer gene, such as, but not limited to, BRCA1 or BRCA2. Additionally, the filter 220 requires that the subject has been diagnosed with breast cancer younger than a predetermined age. In typical embodiments, the predetermined age is fifty years old. In some embodiments, the predetermined age is thirty years old. In some embodiments, the predetermined age is forty years old. In some embodiments, the predetermined age is sixty years old. In such embodiments, a corresponding alert 222 communicates to the medical practitioner device 104, the medical record 206, and/or the subject “This patient has breast cancer and diagnosis age is less than or equal to the age of 50, and has a known genetic mutation within the family.”

Blocks 1086-1090.

In some embodiments, a filter 220 fires when the electronic medical record 206 indicates that the subject has been diagnosed with breast cancer as well as diagnosed as triple negative for estrogen receptor (ER), progesterone receptor (PR), and human epidermal growth factor receptor 2 (HER2) younger than a predetermined age. In typical embodiments, the second predetermined age is sixty years old. In some embodiments, the predetermined age is thirty years old. In some embodiments, the predetermined age is forty years old. In some embodiments, the predetermined age is fifty years old. In such embodiments, a corresponding alert 222 communicates to the medical practitioner device 104, the medical record 206, and/or the subject “This patient has been diagnosed with triple negative breast cancer under, or equal to, the age of 60.”

Blocks 1092-1094.

In some embodiments, a filter 220 fires when the electronic medical record 206 indicates that the subject has been diagnosed with double breast cancer. In such embodiments, a corresponding alert 222 communicates to the medical practitioner device 104, the medical record 206, and/or the subject “This patient has had two breast cancer diagnosis.”

Blocks 1096-1100.

Referring to block 1096-1100 of FIG. 6G, in the method, in some embodiments, a filter 220 fires when the electronic medical record 206 indicates that the subject has been diagnosed with breast cancer and that the other human incurred a diagnosis of breast cancer at or younger than a predetermined age. In typical embodiments, the predetermined age is fifty years old. In some embodiments, the predetermined age is thirty years old. In some embodiments, the predetermined age is forty years old. In some embodiments, the predetermined age is sixty years old. In such embodiments, a corresponding alert 222 communicates to the medical practitioner device 104, the medical record 206, and/or the subject “This patient has breast cancer and one or more first, second, or third degree relatives with diagnosis of breast cancer under or equal to the age of 50.”

Blocks 1102-1104.

In some embodiments, a filter 220 fires when the electronic medical record indicates that the subject has been diagnosed with breast cancer. The filter 220 further requires that the other human has been diagnosed with ovarian cancer. In such embodiments, a corresponding alert 222 communicates to the medical practitioner device 104, the medical record 206, and/or the subject “This patient has breast cancer and one or more first, second, or third degree relatives with diagnosis of breast cancer at any age.”

Blocks 1106-1108.

In some embodiments, a filter 220 fires when the electronic medical record 206 indicates that the subject has been diagnosed with breast cancer. The filter 220 also requires that the other human has been diagnosed with breast cancer or pancreatic cancer. Furthermore, the filter 220 requires that an additional human share a first degree, second degree, or third degree consanguineous relationship with the subject, and has been diagnosed with breast cancer or pancreatic cancer. In such embodiments, a corresponding alert 222 communicates to the medical practitioner device 104, the medical record 206, and/or the subject “This patient has breast cancer and two or more first, second, or third degree relatives with diagnosis of breast cancer.”

Blocks 1110-1112.

In some embodiments, a filter 220 fires when the electronic medical record 206 indicates that the subject (e.g., personal record 208) has been diagnosed with breast cancer and pancreatic cancer. In such embodiments, a corresponding alert 222 communicates to the medical practitioner device 104, the medical record 206, and/or the subject “This patient has breast cancer and a diagnosis of pancreatic cancer.”

Blocks 1114-1116.

In some embodiments, a filter 220 fires when the electronic medical record 206 indicates that the subject (e.g., personal record 208) has been diagnosed with breast cancer and is from a predetermined population. Typically, the population comprises Ashkenazi Jewish, Icelandic, Finnish, Dutch, Norwegian, Scottish/Irish, and African-American. However, the present disclosure is not limited thereto. In some embodiments, additional ancestry to subject health associations are formed and incorporated into the filter 220. In such embodiments, a corresponding alert 222 communicates to the medical practitioner device 104, the medical record 206, and/or the subject “This patient has breast cancer and from a population at increased risk (Ashkenazi Jewish, Icelandic, Finnish, Dutch, Norwegian, Scottish/Irish, and African-American).”

Blocks 1118-1120.

Referring to block 1118-1120 of FIG. 6H, in the method, in some embodiments, a filter 220 fires when the electronic medical record 206 indicates that the subject has been diagnosed with breast cancer and ovarian cancer. In such embodiments, a corresponding alert 222 communicates to the medical practitioner device 104, the medical record 206, and/or the subject “This patient has breast cancer and a diagnosis of ovarian cancer.”

Blocks 1122-1124.

In some embodiments, a filter 220 fires when the electronic medical record 206 indicates that the subject has been diagnosed with breast cancer and is a male. In such embodiments, a corresponding alert 222 communicates to the medical practitioner device 104, the medical record 206, and/or the subject “This patient has a personal history of male breast cancer.”

Blocks 1126-1128.

In some embodiments, a filter 220 fires when the electronic medical record 206 indicates that the subject does not have breast cancer while the other human has a known cancer susceptibility gene. In such embodiments, a corresponding alert 222 communicates to the medical practitioner device 104, the medical record 206, and/or the subject “This patient has a relative with a known cancer susceptibility gene.”

Blocks 1130-1132.

In some embodiments, a filter 220 fires when the electronic medical record 206 indicates that the subject does not have breast cancer while the other human has two or more breast cancers. In such embodiments, a corresponding alert 222 communicates to the medical practitioner device 104, the medical record 206, and/or the subject “This patient has one or more relatives with two or more breast cancers in a single individual.”

Blocks 1134-1138.

In some embodiments, a filter 220 fires when the electronic medical record 206 indicates that the subject does not have breast cancer while the other human incurred a breast cancer diagnosis prior to a predetermined age. The filter 220 also requires that an additional human, who has been diagnosed with breast cancer, share a first degree, second degree, or third degree consanguineous relationship with the subject, while also being on the same side of the family as the other human with respect to the subject (e.g., a common paternal ancestry. In typical embodiments, the third predetermined age is fifty years old. In such embodiments, a corresponding alert 222 communicates to the medical practitioner device 104, the medical record 206, and/or the subject “This patient has as one or more relatives with breast cancer on a family side with at least one diagnosed under the age of 50.”

Blocks 1140-1142.

Referring to blocks 1140-1142 of FIG. 6I, in the method, in some embodiments, a filter 220 fires when the electronic medical record 206 indicates that the subject does not have breast cancer. The filter 220 also requires and that the other human has ovarian cancer. In such embodiments, a corresponding alert 224 communicates to the medical practitioner device 104, the medical record 206, and/or the subject “This patient has one or more relatives with ovarian cancer.”

Blocks 1144-1146.

In some embodiments, a filter 220 fires when the electronic medical record 206 indicates that the subject does not have breast cancer and that the other human is male and has breast cancer. In such embodiments, a corresponding alert 222 communicates to the medical practitioner device 104, the medical record 206, and/or the subject “This patient has one or more relatives with male breast cancer.”

Blocks 1148-1152.

In some embodiments, a filter 220 fires when the electronic medical record 206 indicates that the subject does not have breast cancer. The filter 220 also requires that the other human incurred a diagnosis for breast cancer at a predetermined age. In typical embodiments, the predetermined age is forty-five years old. In some embodiments, the predetermined age is thirty years old. In some embodiments, the predetermined age is fifty years old. In some embodiments, the predetermined age is sixty years old. In some embodiments, the predetermined age is seventy-five years old. In such embodiments, a corresponding alert 220 communicates to the medical practitioner device 104, the medical record 206, and/or the subject “This patient has one or more relatives with breast cancer diagnosed under or equal to the age of 45.”

Blocks 1154-1156.

In some embodiments, a filter 220 fires when the electronic medical record 206 indicates that the subject does not have breast cancer. The filter 220 also requires that there are three more humans which each having at least a third degree consanguineous relationship with the subject. Each of the three humans independently have at least one condition of breast cancer, pancreatic cancer, prostate cancer, melanoma, sarcoma, adrenocortical carcinoma, brain tumor, leukemia, diffuse gastric cancer, colon cancer, endometrial cancer, thyroid cancer, kidney cancer, a dermatologic manifestation a macrocephaly, and hamartomatous polyps of GI tract. However, the present disclosure is not limited thereto. In such embodiments, a corresponding alert 222 communicates to the medical practitioner device 104, the medical record 206, and/or the subject “This patient has a family history of three or more: breast, pancreatic cancer, prostate cancer, melanoma, sarcoma, adrenocortical carcinoma, brain tumors, leukemia, diffuse gastric cancer, colon cancer, endometrial cancer, thyroid cancer, kidney cancer, dermatologic manifestation and/or macrocephaly, and hamartomatous polyps of GI tract.”

Block 1158.

In some embodiments, filters are categorized as a recommendation or a calculation. In typical embodiments, filters categorized as a recommendation are prioritized over, and thus run before, filters categorized as a calculation. However, the present disclosure is not limited thereto. In some embodiments, filters categorized as a calculation are prioritized over, and thus ran before, filters categorized as a calculation. In some embodiments, each filter in the plurality of filters is denoted by a weighted value, and the plurality of filters are executed according an arrangement of the weighted values. Likewise, in other embodiments, each filter in the plurality of filters is denoted by a tier value, and the plurality of filters are executed according to an arrangement of the tier values. The above prioritizations are not limited to specific filters, but could embody filter sets in the plurality of filter sets.

In addition to the above coded filters, or rules, a decision engine must be formed. The JAVA code, as shown in FIGS. 8D through 8F, is an exemplary embodiment of such a service.

In many embodiments, the referenced CdsDecision is held on a cloud server, such as a EC2 cloud, however, the present disclosure is not limited thereto. In some embodiments, the decision engine is held locally on a device.

Block 1160.

Referring to block 1160 of FIG. 6J, in the method when a respective filter in the corresponding filter set is fired, the subject is deemed to have an actionable risk of the hereditary cancer represented by the corresponding filter set. In some embodiments, when a predetermined condition or data item is met and/or parsed, no actionable risk of hereditary cancer is found and the alerts are suppressed.

Blocks 1162-1164.

Referring to blocks 1162-1164, in some embodiments communication of an alert 222 is initiated when the respective filter set (e.g., filter set 218-1) in the plurality of filter sets that includes the corresponding filter 220 is fired. In some embodiments, the alert 220 is communicated as an email to the medical practitioner device 104, the medical record 206, and/or the subject, a text message to the medical practitioner device 104 and/or the subject, a computer generated audio message to the medical practitioner device 104, the medical record 206, and/or the subject, an input in the EMR 206 of the subject (e.g., data 210 of FIG. 2), or a similar form of communication. For instance, in one implementation the alert 222 communicates an email to the medical practitioner device 104 and/or the subject which includes a message in accordance with the corresponding fired filter 220.

It should be understood that the particular order in which the operations in FIGS. 6A-6J have been described is merely an example and is not intended to indicate that the described order is the only order in which the operations could be performed. One of ordinary skill in the art would recognize various ways to reorder the operations described herein. Additionally, it should be noted that details of other processes described herein with respect to other methods described herein (e.g., method 900) are also applicable in an analogous manner to method 600 described above with respect to FIGS. 6A-6J. Further, in some embodiments, method 600 can be used in conjunction with any other method described herein (e.g., method 900). The operations in the information processing methods described above are, optionally implemented by running one or more functional modules in information processing apparatus such as general purpose processors (e.g., as described above with respect to FIGS. 2-4) or application specific chips.

FIG. 9 is a flow diagram illustrating a method 900 for determining whether a first human has an actionable risk for each of a plurality of hereditary cancers. Some or all portions of method 900 are performed at a computer system (e.g., computer system 200 or 250 in FIGS. 2 and 3) having one or more processors, and memory storing one or more programs for execution by the one or more processors for determining whether a first human has an actionable risk for each of a plurality of hereditary cancers. Some operations in method 900 are, optionally, combined and/or the order of some operations is, optionally, changed.

In some embodiments, method 900 is at least partially performed at a computer system comprising one or more processors, and memory storing one or more programs for execution by the one or more processors. The method includes obtaining (902) medical information about a subject. For example, in some embodiments, responsive to a request, e.g., prior to or following a medical consultation of the first human, by a medical practitioner, an electronic medical record associated with the first human is obtained via a computer system having a processor programmed to receive medical records. The electronic medical record of the first human include a plurality of data items, wherein the plurality of data items includes one or more of an age of the first human, whether the first human has been diagnosed with a cancer, and an indication of whether a second human has been afflicted with a cancer, wherein the second human shares a first degree, second degree, or third degree consanguineous relationship with the first human. In some embodiments, the entirety of an electronic medical record (EMR) is not retrieved or accessed but, rather, specific queries are made of a medical records database and requested information from the EMR of the subject is transmitted from the medical records database. In some embodiments, the information query is performed in anticipation of a medical consultation. In some embodiments, the information query is performed following a medical consultation or procedure, e.g., after the electronic medical record of the subject has been updated to reflect the medical consultation or procedure.

In some embodiments, the medical information includes information related to a hereditary cancer status of the subject and/or a family member, e.g., hereditary non-polyposis colorectal cancer (HNPCC) related cancers, hereditary breast and ovarian cancer syndrome (HBOC) related cancer, Cowden syndrome related cancers, and/or Li-Fraumeni syndrome related cancers. That is, the system queries the medical records database for any medical information in the subject's medical record related to these cancer statuses. The output from the medical database, therefore, includes information about these cancer status regardless of whether diagnoses are included in the medical records. That is, the lack of information about a cancer diagnosis is indicative that the subject and/or family member has not been diagnosed with a hereditary cancer.

Method 900 then includes applying (904) the medical information, e.g., about the subject and/or family members, against a first filter set associated with one or more hereditary cancers. Method 900 also includes applying (906) the medical information, e.g., about the subject and/or family members, against a second filter set associated with one or more hereditary cancers. Generally, each respective filter of a filter set is used to identify conditions (e.g., medical information about the subject and/or family members) that indicate the subject has a higher probability (e.g., as compared to the probability in the general population) of carrying an allele associated with an increased risk of developing a hereditary cancer.

For example, in some embodiments, for each respective hereditary cancer in the plurality of hereditary cancers, the method includes applying an algorithm to the plurality of data items, via a computer system having a processor programmed to perform the algorithm, where the algorithm runs the plurality of data items against a corresponding filter set in a plurality of filter sets. each respective filter set in the plurality of filter sets represents a different hereditary cancer in the plurality of hereditary cancers. For example, in some embodiments, a first filter set is associated with identifying risk factors for a hereditary non-polyposis colorectal cancer (HNPCC) related cancer and a second filter set is associated with identifying risk factors for a hereditary breast and ovarian cancer syndrome (HBOC) related cancer. Each respective filter set in the plurality of filter sets includes respective filters that are configured to be fired at least when the plurality of data items indicates the first human has a risk factor for the corresponding hereditary cancer.

For example, in some embodiments, the medical information is applied against a first filter set associated with identifying risk factors for a hereditary non-polyposis colorectal cancer (HNPCC) related cancer. In some embodiments, the first filter set includes one or more filters selected from the filters listed in Table 1, shown below. E.g., in some embodiments, the first filter set includes any 1, 2, 3, 4, 5, 6, 7, 8, 9, or all 10 of the filters listed in Table 1.

TABLE 1
Example HNPCC related cancer filters.

| Filter | Conditions of Filter |
|---|---|
| 1a | (i) subject has not been diagnosed with colorectal |
| | cancer or endometrial cancer, |
| | (ii) first degree consanguineous relationship |
| | (iii) premature familial colorectal or endometrial |
| | cancer diagnosis |
| 2a | (i) subject has not been diagnosed with colorectal |
| | cancer or endometrial cancer |
| | (ii) first degree consanguineous relationship |
| | (iii) familial diagnosis of colorectal or endometrial |
| | cancer and another HNPCC related cancer |
| 3a | (i) subject has not been diagnosed with colorectal |
| | cancer or endometrial cancer |
| | (ii) first degree or second degree consanguineous |
| | relationship |
| | (iii) two familial HNPCC related cancer diagnoses |
| | (iv) at least one premature diagnosis |
| 4a | (i) subject has not been diagnosed with colorectal |
| | cancer or endometrial cancer |
| | (ii) first degree or second degree consanguineous |
| | relationship |
| | (iii) three familial HNPCC related cancer diagnoses |
| 5a | (i) subject has not been diagnosed with colorectal |
| | cancer or endometrial cancer |
| | (ii) first degree consanguineous relationship |
| | (iii) familial polyposis diagnosis |
| 6a | (i) subject diagnosed with colorectal cancer |
| | (ii) premature diagnosis |
| 7a | (i) subject diagnosed with endometrial cancer |
| 8a | (i) subject diagnosed with colorectal cancer or |
| | endometrial cancer |
| | (ii) subject diagnosed with another HNPCC |
| | related cancer |
| 9a | (i) subject diagnosed with colorectal cancer or |
| | endometrial cancer |
| | (ii) first degree consanguineous relationship |
| | (iii) premature familial HNPCC related cancer |
| | diagnosis |
| 10a | (i) subject diagnosed with colorectal cancer or |
| | endometrial cancer |
| | (ii) first degree or second degree consanguineous |
| | relationship |
| | (iii) two familial HNPCC related cancer diagnoses |

Accordingly, it is contemplated that in some embodiments the first set of filters includes any sub-set of the filters provided in Table 1. It is also contemplated that any filters provided in Table 2 may be combined and or applied as part of a single filter. Likewise, in some embodiments the skilled artisan will know of other filters, not provided in Table 1, which may be combined with any subset of the filters in Table 1 to form the first filter set used in the method as described herein. For brevity, all possible combinations of the filters provided in Table 1 are not specifically delineated here.

For example, in some embodiments, the first filter set includes a first filter (e.g., filter 1a) that fires when the electronic medical record indicates (i) that the first human has not been diagnosed with colorectal cancer or endometrial cancer, (ii) that the second human shares a first degree consanguineous relationship with the first human, and (iii) that the second human was diagnosed with colorectal or endometrial cancer at an age less than a first predetermined age, e.g., as exemplified in the code shown in FIG. 8.

For example, in some embodiments, the first filter set includes a second filter (e.g., filter 2a) that fires when the electronic medical record indicates (i) that the first human has not been diagnosed with colorectal cancer or endometrial cancer, (ii) that the second human shares a first degree consanguineous relationship with the first human, and (iii) that the second human has been diagnosed with colorectal or endometrial cancer and another HNPCC related cancer in the plurality of HNPCC related cancers other than colorectal or endometrial cancer, e.g., as exemplified in the code shown in FIG. 8.

For example, in some embodiments, the first filter set includes a third filter (e.g., filter 3a) that fires when the electronic medical record indicates (i) that the first human has not been diagnosed with colorectal cancer or endometrial cancer, (ii) that the second human shares a first degree or second degree consanguineous relationship with the first human, (iii) that the second human has been diagnosed with an HNPCC related cancer in the plurality of HNPCC related cancers, (iv) that a third human shares a first degree or second degree consanguineous relationship with the first human, and (v) that the third human was diagnosed with an HNPCC related cancer in the plurality of HNPCC related cancers before a first predetermined age, e.g., as exemplified in the code shown in FIG. 8.

For example, in some embodiments, the first filter set includes a fourth filter (e.g., filter 4a) that fires when the electronic medical record indicates (i) that the first human has not been diagnosed with colorectal cancer or endometrial cancer (ii) that the second human shares a first degree or second degree consanguineous relationship with the first human, (iii) that the second human has been diagnosed with an HNPCC related cancer in the plurality of HNPCC related cancers, (iv) that a third human shares a first degree or second consanguineous relationship with the first human, (v) that the third human has been diagnosed with an HNPCC related cancer in the plurality of HNPCC related cancers, (vi) that a fourth human shares a first degree or second degree consanguineous relationship with the first human, and (vii) that the fourth human has been diagnosed with an HNPCC related cancer in the plurality of HNPCC related cancers, e.g., as exemplified in the code shown in FIG. 8.

For example, in some embodiments, the first filter set includes a fifth filter (e.g., filter 5a) that fires when the electronic medical record indicates (i) that the first human has not been diagnosed with colorectal cancer or endometrial cancer, (ii) that the second human shares a first degree consanguineous relationship with the first human, and (iii) that the second human has been diagnosed with polyposis, e.g., as exemplified in the code shown in FIG. 8.

For example, in some embodiments, the first filter set includes a sixth filter (e.g., filter 6a) that fires when the electronic medical record indicates (i) that the first human has been diagnosed with colorectal cancer and (i) the first human is younger than a first predetermined age, e.g., as exemplified in the code shown in FIG. 8.

For example, in some embodiments, the first filter set includes a seventh filter (e.g., filter 7a) that fires when the electronic medical record indicates that the first human has been diagnosed with endometrial cancer, e.g., as exemplified in the code shown in FIG. 8.

For example, in some embodiments, the first filter set includes an eighth filter (e.g., filter 8a) that fires when the electronic medical record indicates (i) that the first human has been diagnosed with colorectal cancer or endometrial cancer and (ii) that the first human has been diagnosed with another HNPCC related cancer in the plurality of HNPCC related cancers, e.g., as exemplified in the code shown in FIG. 8.

For example, in some embodiments, the first filter set includes a ninth filter (e.g., filter 9a) that fires when the electronic medical record indicates (i) that the first human has colorectal cancer or endometrial cancer, (ii) that the second human shares a first degree consanguineous relationship with the first human, and (iii) that the second human was diagnosed with an HNPCC related cancer in the plurality of HNPCC related cancers at an age less than a first predetermined age, e.g., as exemplified in the code shown in FIG. 8.

For example, in some embodiments, the first filter set includes a tenth filter (e.g., filter 10a) that fires when the electronic medical record indicates (i) that the first human has colorectal cancer or endometrial cancer, (ii) that the second human shares a first degree or second degree consanguineous relationship with the first human, (iii) that the second human has been diagnosed with an HNPCC related cancer in the plurality of HNPCC related cancers, and (iv) that a third human shares a first degree or second degree consanguineous relationship with the first human, and (v) that the third human has been diagnosed with an HNPCC related cancer in the plurality of HNPCC related cancers, e.g., as exemplified in the code shown in FIG. 8.

In some of the above embodiments, the first predetermined age is 50. In other embodiments, the first predetermined age is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, etc.

In some embodiments, the plurality of data items further includes an indication that the first human has a lab order for a genetic test or has the results for a genetic test within a predetermined threshold period, and the method includes suppressing the firing of each respective filter in each filter set in the plurality of filter sets. In some embodiments, the threshold period is one year. In other embodiments, the threshold period is 1, 2, 3, 4, 5, 6, 7, 8, 9, 10, 11, 12, 13, 14, 15, 16, 17, 18, 19, 20, 21, 22, 23, 24, or more months.

In some embodiments, the plurality of HNPCC related cancers consists of colorectal cancer, endometrial/uterine cancer, stomach cancer, urinary tract cancer, small bowel cancer, brain cancer, ovarian cancer, liver cancer, kidney cancer, bile duct cancer, prostate cancer, pancreatic cancer, and breast cancer. For more information on hereditary non-polyposis colorectal cancer see, for example, Yurgelun M B, Hampel H. Recent Advances in Lynch Syndrome: Diagnosis, Treatment, and Cancer Prevention, Am Soc Clin Oncol Educ., (38):101-09 (2018), the content of which is incorporated herein by reference, in its entirety, for all purposes.

In some embodiments, the plurality of data items includes a diagnosis that the first human has a family history of HNPCC, and the method includes suppressing the firing of each respective filter in each filter set in the plurality of filter sets.

In some embodiments, the plurality of data items includes a diagnosis that the first human has HNPCC, and the method includes suppressing the firing of each respective filter in each filter set in the plurality of filter sets.

In some embodiments, the first human is deemed to have a diagnosis for HNPCC when the plurality of data items includes a Systematized Nomenclature of Medicine Clinical Term value of 699346009, 315058005, 459528015, 2971230015, or 459527013.

Similarly, in some embodiments, the medical information is applied against a second filter set associated with identifying risk factors for a hereditary breast and ovarian cancer syndrome (HBOC) related cancer. In some embodiments, the first filter set includes one or more filters selected from the filters listed in Table 2, shown below. E.g., in some embodiments, the first filter set includes any 1, 2, 3, 4, 5, 6, 7, 8, 9, 10, 11, 12, 13, 14, 15, 16, 17, 18, 19, or all 20 of the filters listed in Table 2.

TABLE 2
Example HBOC related cancer filters.

| Filter | Conditions of Filter |
|---|---|
| 1b | (i) subject has been diagnosed with breast cancer |
| | (ii) first degree, second degree, or third degree |
| | consanguineous relationship |
| | (iii) familial carrier of HBOC related cancer |
| | susceptibility gene |
| 2b | (i) subject has been diagnosed with ovarian cancer |
| 3b | (i) subject has been diagnosed with breast cancer |
| | (ii) premature diagnosis |
| 4b | (i) subject has been diagnosed with triple negative |
| | breast cancer |
| | (ii) premature diagnosis |
| 5b | (i) subject has been twice diagnosed with breast cancer |
| 6b | (i) subject has been diagnosed with breast cancer |
| | (ii) first degree, second degree, or third degree |
| | consanguineous relationship |
| | (iii) premature familial breast cancer diagnosis |
| 7b | (i) subject diagnosed with breast cancer |
| | (ii) first degree, second degree, or third degree |
| | consanguineous relationship |
| | (iii) familial ovarian cancer diagnosis |
| 8b | (i) subject has been diagnosed with breast cancer |
| | (ii) first degree, second degree, or third degree |
| | consanguineous relationship |
| | (iii) premature familial breast cancer diagnosis |
| 9b | (i) subject has been diagnosed with breast cancer |
| | (ii) first degree, second degree, or third degree |
| | consanguineous relationship |
| | (iii) two familial breast cancer or pancreatic |
| | cancer diagnoses |
| 10b | (i) subject has been diagnosed with breast cancer |
| | (ii) subject has been diagnosed with pancreatic cancer |
| 11b | (i) subject has been diagnosed with breast cancer |
| | (ii) Ashkenazi Jewish, Icelandic, Finnish, Dutch, |
| | Norwegian, Scottish, Irish, or African-American decent |
| 12b | (i) subject has been diagnosed with breast cancer |
| | (ii) subject has been diagnosed with ovarian cancer |
| 13b | (i) subject has been diagnosed with breast cancer |
| | (ii) subject is male |
| 14b | (i) subject has not been diagnosed with breast cancer |
| | (ii) first degree, second degree, or third degree |
| | consanguineous relationship |
| | (iii) familial carrier of HBOC related cancer |
| | susceptibility gene |
| 15b | (i) subject has not been diagnosed with breast cancer |
| | (ii) first degree, second degree, or third degree |
| | consanguineous relationship |
| | (iii) familial history of twice diagnosed breast cancer |
| 16b | (i) subject has not been diagnosed with breast cancer |
| | (ii) first degree, second degree, or third degree |
| | consanguineous relationship |
| | (iii) premature familial breast cancer diagnosis |
| | (iv) second familial breast cancer diagnosis |
| 17b | (i) subject has not been diagnosed with breast cancer |
| | (ii) first degree, second degree, or third degree |
| | consanguineous relationship |
| | (iii) familial ovarian cancer diagnosis |
| 18b | (i) subject has not been diagnosed with breast cancer |
| | (ii) first degree, second degree, or third degree |
| | consanguineous relationship |
| | (iii) familial male breast cancer diagnosis |
| 19b | (i) subject has not been diagnosed with breast cancer |
| | (ii) first degree or second degree consanguineous |
| | relationship |
| | (iii) premature familial breast cancer diagnosis |
| 20b | (i) subject has not been diagnosed with breast cancer |
| | (ii) first degree, second degree, or third degree |
| | consanguineous relationship |
| | (iii) three familial diagnoses of breast cancer, pancreatic |
| | cancer, prostate cancer, melanoma, sarcoma, adrenocortical |
| | carcinoma, brain tumor, leukemia, diffuse gastric cancer, |
| | colon cancer, endometrial cancer, thyroid cancer, kidney |
| | cancer, a dermatologic manifestation a macrocephaly, or |
| | hamartomatous polyps of GI tract |

Accordingly, it is contemplated that in some embodiments the first set of filters includes any sub-set of the filters provided in Table 2. It is also contemplated that any filters provided in Table 2 may be combined and or applied as part of a single filter. E.g., the combination of filters 1b and 14b into a single filter requiring (i) a first degree, second degree, or third degree consanguineous relationship, and (ii) a familial carrier of HBOC related cancer susceptibility gene. Likewise, in some embodiments the skilled artisan will know of other filters, not provided in Table 2, which may be combined with any subset of the filters in Table 2 to form the first filter set used in the method as described herein. For brevity, all possible combinations of the filters provided in Table 2 are not specifically delineated here.

For example, in some embodiments, the first filter set includes a first filter (e.g., filter 1b) that fires when the electronic medical record indicates (i) that the first human has been diagnosed with breast cancer, (ii) that the second human shares a first degree, second degree, or third degree consanguineous relationship with the first human, and (iii) that the second human carries an HBOC related cancer susceptibility gene, e.g., as exemplified in the code shown in FIG. 8.

For example, in some embodiments, the first filter set includes a second filter (e.g., filter 2b) that fires when the electronic medical record indicates that the first human has been diagnosed with ovarian cancer, e.g., as exemplified in the code shown in FIG. 8.

For example, in some embodiments, the first filter set includes a third filter (e.g., filter 3b) that fires when the electronic medical record indicates (i) that the first human has been diagnosed with breast cancer, and (ii) that the first human no older than a second predetermined age when diagnosed with breast cancer, e.g., as exemplified in the code shown in FIG. 8.

For example, in some embodiments, the first filter set includes a fourth filter (e.g., filter 4b) that fires when the electronic medical record indicates (i) that the first human has been diagnosed with breast cancer, (ii) that the breast cancer of the first human has been diagnosed as triple negative for estrogen receptor (ER), progesterone receptor (PR), and human epidermal growth factor receptor 2 (HER2), and (iii) that the first human was no older than a third predetermined age when diagnosed with triple negative breast cancer, e.g., as exemplified in the code shown in FIG. 8.

For example, in some embodiments, the first filter set includes a fifth filter (e.g., filter 5b) that fires when the electronic medical record indicates that the first human has been diagnosed with breast cancer twice, e.g., as exemplified in the code shown in FIG. 8.

For example, in some embodiments, the first filter set includes a sixth filter (e.g., filter 6b) that fires when the electronic medical record indicates (i) that the first human has been diagnosed with breast cancer, (ii) that the second human shares a first degree, a second degree, or a third degree consanguineous relationship with the first human, and (iii) that the second human was diagnosed with a breast cancer at an age of no more than a second predetermined age, e.g., as exemplified in the code shown in FIG. 8.

For example, in some embodiments, the first filter set includes a seventh filter (e.g., filter 7b) that fires when the electronic medical record indicates (i) that the first human has been diagnosed with breast cancer, (ii) that the second human shares a first degree, a second degree, or a third degree consanguineous relationship with the first human, and (iii) that the second human has been diagnosed with ovarian cancer, e.g., as exemplified in the code shown in FIG. 8.

For example, in some embodiments, the first filter set includes an eighth filter (e.g., filter 8b) that fires when the electronic medical record indicates (i) that the first human has been diagnosed with breast cancer, (ii) that the second human shares a first degree, a second degree, or a third degree consanguineous relationship with the first human, and (iii) that the second human was diagnosed with a breast cancer at an age of no more than a second predetermined age, e.g., as exemplified in the code shown in FIG. 8.

For example, in some embodiments, the first filter set includes a ninth fifth filter (e.g., filter 9b) that fires when the electronic medical record indicates (i) that the first human has been diagnosed with breast cancer, (ii) that the second human shares a first degree, a second degree, or a third degree consanguineous relationship with the first human, (iii) that the second human was diagnosed with breast cancer or pancreatic cancer, (iv) that a third human shares a first degree, a second degree, or a third degree consanguineous relationship with the first human, and (v) that the third human was diagnosed with breast cancer or pancreatic cancer, e.g., as exemplified in the code shown in FIG. 8.

For example, in some embodiments, the first filter set includes a tenth filter (e.g., filter 10b) that fires when the electronic medical record indicates (i) that the first human has been diagnosed with breast cancer, and (ii) that the first human has been diagnosed with pancreatic cancer, e.g., as exemplified in the code shown in FIG. 8.

For example, in some embodiments, the first filter set includes an eleventh filter (e.g., filter 11b) that fires when the electronic medical record indicates (i) that the first human has been diagnosed with breast cancer, and (ii) that the first human is from a population selected from the group consisting of Ashkenazi Jewish, Icelandic, Finnish, Dutch, Norwegian, Scottish, Irish, and African-American, e.g., as exemplified in the code shown in FIG. 8.

For example, in some embodiments, the first filter set includes a twelfth filter (e.g., filter 12b) that fires when the electronic medical record indicates (i) that the first human has been diagnosed with breast cancer, and (ii) that the first human has been diagnosed with ovarian cancer, e.g., as exemplified in the code shown in FIG. 8.

For example, in some embodiments, the first filter set includes a thirteenth filter (e.g., filter 13b) that fires when the electronic medical record indicates (i) that the first human has been diagnosed with breast cancer, and (ii) that the first human is male, e.g., as exemplified in the code shown in FIG. 8.

For example, in some embodiments, the first filter set includes a fourteenth filter (e.g., filter 14b) that fires when the electronic medical record indicates (i) that the first human has not been diagnosed with breast cancer, (ii) that the second human shares a first degree, second degree, or third degree consanguineous relationship with the first human, and (iii) that the second human carries an HBOC related cancer susceptibility gene, e.g., as exemplified in the code shown in FIG. 8. In some embodiments, the second human is deemed to carry an HBOC related cancer susceptibility gene when the plurality of data items includes a Systematized Nomenclature of Medicine Clinical Term value of 445333001 or 85101000119100 for the second human.

For example, in some embodiments, the first filter set includes a fifteenth filter (e.g., filter 15b) that fires when the electronic medical record indicates (i) that the first human has not been diagnosed with breast cancer, (ii) that the second human shares a first degree, second degree, or third degree consanguineous relationship with the first human, and (iii) that the second human has been diagnosed with breast cancer twice, e.g., as exemplified in the code shown in FIG. 8.

For example, in some embodiments, the first filter set includes a sixteenth filter (e.g., filter 16b) that fires when the electronic medical record indicates (i) that the first human does not have breast cancer, (ii) that the second human shares a first degree, second degree, or third degree consanguineous relationship with the first human, (iii) that the second human was diagnosed with breast cancer at an age of nor more than a second predetermined age, (iv) that a third human shares a first degree, second degree, or third degree consanguineous relationship with the first human, (v) that the third human is on the same side of the family as the second human with respect to the first human, and (vi) that the third human has been diagnosed with breast cancer, e.g., as exemplified in the code shown in FIG. 8.

For example, in some embodiments, the first filter set includes a seventeenth filter (e.g., filter 17b) that fires when the electronic medical record indicates (i) that the first human does not have breast cancer, (ii) that the second human shares a first degree, second degree, or third degree consanguineous relationship with the first human, (iii) that the second human has been diagnosed with ovarian cancer, e.g., as exemplified in the code shown in FIG. 8.

For example, in some embodiments, the first filter set includes an eighteenth filter (e.g., filter 18b) that fires when the electronic medical record indicates (i) that the first human does not have breast cancer, (ii) that the second human shares a first degree, second degree, or third degree consanguineous relationship with the first human, (iii) that the second human has been diagnosed with male breast cancer, e.g., as exemplified in the code shown in FIG. 8.

For example, in some embodiments, the first filter set includes a nineteenth filter (e.g., filter 19b) that fires when the electronic medical record indicates (i) that the first human does not have breast cancer, (ii) that the second human shares a first degree or second degree consanguineous relationship with the first human, (iii) that the second human was diagnosed with breast cancer at an age of no more than a fourth predetermined age, e.g., as exemplified in the code shown in FIG. 8.

For example, in some embodiments, the first filter set includes a twentieth filter (e.g., filter 20b) that fires when the electronic medical record indicates (i) that the first human does not have breast cancer, (ii) that there are three more humans, each sharing a first degree, second degree, or third degree consanguineous relationship with the first human, and (iii) that each of the three or more humans have been diagnosed with a condition selected from the group consisting of breast cancer, pancreatic cancer, prostate cancer, melanoma, sarcoma, adrenocortical carcinoma, brain tumor, leukemia, diffuse gastric cancer, colon cancer, endometrial cancer, thyroid cancer, kidney cancer, a dermatologic manifestation a macrocephaly, and hamartomatous polyps of GI tract, e.g., as exemplified in the code shown in FIG. 8. For more information on hereditary breast and ovarian cancer syndrome (HBOC) see, for example, Petrucelli N. et al., “BRCA1- and BRCA2-Associated Hereditary Breast and Ovarian Cancer,” Gene Reviews, University of Washington, Dec. 15, 2016, the content of which is incorporated herein by reference, in its entirety, for all purposes.

In some of the embodiments above, the second predetermined age is 50 years old. In other embodiments, the second predetermined age is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, etc.

In some of the embodiments above, the third predetermined age is 60 years old. In other embodiments, the third predetermined age is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, etc.

In some of the embodiments above, the fourth predetermined age is 45 years old. In other embodiments, the third predetermined age is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, etc.

In some embodiments, each respective filter set in the plurality of filter sets includes at least one filter the is configured to be fired at least when the plurality of data items indicates the first human has a familial risk factor for the corresponding hereditary cancer that satisfies a first threshold level of consanguinity.

In some embodiments, method 900 includes determining (908) whether a filter has been fired. In no filter was fired, the method includes not making a recommendation (910) for genetic screening (e.g., recommending that the subject does not undergo genetic screening for an allele associated with an increased risk of a hereditary cancer). In some embodiments, when no filters in a corresponding filter set are fired, the first human is deemed to not have an actionable risk of the hereditary cancer.

If a filter was fired, the method includes making a recommendation that the subject undergo genetic screening for an allele associated with an increased risk of a hereditary cancer. In some embodiments, the recommendation is sent in the form of an alert. In some embodiments, the alert is in the form of a recommendation or a calculation. In some embodiments, the alert includes a recitation of a data item in the plurality of data items. In some embodiments, alerts that are recommendations are prioritized over alerts that are calculations. In some embodiments, the alert is in the form of an email, a text message, a document, an audio signal, an entry in the electronic medical record, or a combination thereof.

In some embodiments, where a filter was fired, method 900 includes performing (914) the genetic screening. In some embodiments, the method also includes taking action (916) on the results of the genetic screening.

It should be understood that the particular order in which the operations in FIG. 9 have been described is merely an example and is not intended to indicate that the described order is the only order in which the operations could be performed. One of ordinary skill in the art would recognize various ways to reorder the operations described herein. Additionally, it should be noted that details of other processes described herein with respect to other methods described herein (e.g., method 600) are also applicable in an analogous manner to method 900 described above with respect to FIG. 9. Further, in some embodiments, method 900 can be used in conjunction with any other method described herein (e.g., method 600). The operations in the information processing methods described above are, optionally implemented by running one or more functional modules in information processing apparatus such as general purpose processors (e.g., as described above with respect to FIGS. 2-4) or application specific chips.

Example Medical Conditions

In some embodiments, the goal of one aspect of the present disclosure is to monitor a subject for a disease condition such as irritable bowel disease (IBD). For instance, in some embodiments, the goal of one aspect of the present disclosure is to monitor a subject for a condition described by the SNOMED-CT value 24526004, 22207007, 34000006, 426549001, 413276006, 397172008, 397173003, 1085901000119101, 1085751000119100, 1085801000119106, 1085851000119105, 7620006, 50440006, 3815005, 71833008, 414153008, 56287005, 38106008, 196977009, 91390005, 414154002, 359664009, 359656003, 235607002, 235664007, 70622003, 61424003, 64766004, 10928410, 00119100, 10851310, 00119100, 10852310, 00119100, 128600008, 444546002, 13470001, 442159003, 52506002, 78324009, 78712000, 14311001, 414156000, 410484008, 445243001, 441971007, 697969008, and/or 444548001. In some embodiments, the goal of one aspect of the present disclosure is to monitor a subject for a condition described by any combination of such SNOMED-CT values. In some embodiments, the goal of one aspect of the present disclosure is lynch syndrome. For instance, in some embodiments, the goal of one aspect of the present disclosure is to monitor a subject for a condition described by the SNOMED-CT value 716318002. In some embodiments, the goal of one aspect of the present disclosure is a family history of lynch syndrome. For instance, in some embodiments, the goal of one aspect of the present disclosure is to monitor a subject for a condition described by the SNOMED-CT value 1098871000119109.

In some embodiments, the goal of one aspect of the present disclosure is endometrial cancer. For instance, in some embodiments, the goal of one aspect of the present disclosure is to monitor a subject for a condition described by the SNOMED-CT value 123845008, 188192002, 254878006, 107791000119107, 107771000119106, 93781006, 699356008, 699358009, 699357004, 107751000119102, 447266004, 94281004, 727212012, 192334017, 205166013, 571453011, 289190019, 645833015, 379735014, 3287875016, 3287940015, 837587018, 510605012, 3012523015, 2984051018, 2983988013, 3078433017, 2984007010, 2984017017, 2983996015, 2984010015, 3287629014, 3287687013, 3287682019, 3287662018, 2881235017, 2883566019, 838192013, 156021017, 156022012, 371973000, 446022000, 371972005, 188195000, 369493004, 369579009, 369494005, 369495006, 369496007, 10708511000119108, 25477001, 946655001, 309245001, 449073009, 188190005, 188189001, 188192002, 188196004, 188193007, 371971003, 94215000, 447390000, 702369008, 93844001, 372024009, 723077004, 447389009, 447266004, 838112014, 155841016, 155842011, 155844012, 155843018, 371972005, 1197265012, 1210561014, 1229104018, 1218022011, 1216532017, 371973000, 1197266013, 1210562019, 1229105017, 3036191014, 1218023018, 1216533010, 3036277011, 126909004, 730934010, 197018, 126908007, 730933016, 196010, 473932011, 473933018, 92788005, 836384019, 153406013, 3036511019, 123841004, 727206019, 192330014, 1217417017, 1215914017, 92470003, 92787000, 423973006, 160299006, 672281000119109, 428941002, and/or 126915004. In some embodiments, the goal of one aspect of the present disclosure is to monitor a subject for a condition described by any combination of such SNOMED-CT values.

In some embodiments, the goal of one aspect of the present disclosure is colorectal cancer. For instance, in some embodiments, the goal of one aspect of the present disclosure is to monitor a subject for a condition described by the SNOMED-CT value 126847008, 126838000, 109838007, 312112002, 312113007, 312114001, 312115000, 314965007, 363406005, 363407001, 363408006, 363409003, 363410008, 363412000, 363413005, 363414004, 363510005, 425178004, 449218003, 93683002, 93761005, 93771007, 93826009, 93980002, 94006002, 94072004, 94105000, 94179005, 94260004, 94271003, 94328005, 94509004, 94538001, 94604000, 94643001, 94105000, 94179005, 94260004, 94271003, 94328005, 94509004, 94538001, 94604000, 94643001, 133751000119102, 1701000119104, 837563015, 510572019, 662407016, 403809019, 570967019, 288670017, 459528015, 2971230015, 459527013, 13531000119117, 2915311014, 408645001, 2152477017, 2160192016, 9.9111E+13, 3285527015, 3285526012, 837563015, 510572019, 93854002, 837675017, 510741016, 371992003, 93984006, 837832014, 510954015, 363351006, 713573006, 9.6281E+13, 93980002, 837827013, 510950012, 269544008, 662419012, 403829018, 3036985011, 3037113016, 363508008, 755340019, 482803010, 1228621019, 1228618016, 1228620018, 1228616017, 1228619012, 1228617014, 482802017, 428905002, 2689992014, 2693067015, 448675008, 2897596011, 2901970012, 2901969011, 838067013, 155753013, 155754019, 838166018, 155967013, 155970012, 94365007293013, 156249017, 3036520011, 1235836010, 156252013, 94346004, 838270019, 156195015, 156198018, 94313005, 838231013, 156111011, 511257013, 156114015, 1495525014, 3285556015, 3285543010, 276815004, 669776012, 413127011, 413128018, 449072004, 2897664013, 2901944011, 2901945012, 708747014, 455778014, 708746017, 455777016, 708748016, 455779018, 312111009, 708745018, 455776013, 708750012, 455780015, 755228017, 482613016, 1228537017, 3288712016, 482614010, 755229013, 482616012, 1228538010, 482615011, 3288737014, 755230015, 482617015, 3289030012, 482618013, 755231016, 482620011, 482619017, 3288335015, 755233018, 482624019, 3288993017, 482623013, 755234012, 482625018, 1228542013, 482626017, 3288598016, 755236014, 482628016, 482627014, 3288987013, 2639834014, 2643851011, 2897917011, 2902237012, 837468010, 510451019, 837641018, 510695019, 837827013, 510950012, 837939016, 511081012, 838248017, 156153012, 156154018, 838180013, 155995013, 155996014, 838467015, 156619014, 3036192019, 156620015, 838502013, 156687015, 156688013, 838583017, 156839010, 156840012, 838630018, 156933012, 156934018, 837979013, 511132012, 838067013, 155753013, 155754019, 838180013, 155995013, 155996014, 838467015, 156619014, 3036192019, 156620015, 838502013, 156687015, 156688013, 126837005, 126769007, 276816003, 92617001, 363508008, 126837005, 126832004, 94878004, 413214004, 254532005, 109850001, 255077007, 126850006, 126853008, 126769007, 448908007, 254533000, 92568009, 429699009, and/or 126842002}. In some embodiments, the goal of one aspect of the present disclosure is to monitor a subject for a condition described by any combination of such SNOMED-CT values.

In some embodiments, the goal of one aspect of the present disclosure is biliary tract cancer. For instance, in some embodiments, the goal of one aspect of the present disclosure is to monitor a subject for a condition described by the SNOMED-CT value 92530007, 92545000, 92570000, 92574009, 92589000, 92599005, 92618006, 94349006, 109842005, 187773007, 187776004, 187777008, 187784000, 189243008, 189246000, 253017000, 255087006, 314962005, 363353009, 363415003, 363416002, 372139008, 372140005, 447109003, 702712006, 9541000119105, 94185003, 94291005, 94262007, 94164003, 94270002, 94312000, 94349006. In some embodiments, the goal of one aspect of the present disclosure is to monitor a subject for a condition described by any combination of such SNOMED-CT values.

In some embodiments, the goal of one aspect of the present disclosure is pancreatic cancer. For instance, in some embodiments, the goal of one aspect of the present disclosure is to monitor a subject for a condition described by the SNOMED-CT value 363418001, 713189001, 314964006, 187798008, 187791002, 254611009, 255088001, 363419009, 187792009, 372003004, 372142002, 254609000, 363368005, 254612002, 326072005, 363369002, 109848009, 93715005, 372119009, 93843007, 93939009, 371967001, 94082003, 94459006, 285614004, 94212002, 94325008, 94354002, 94460001, 94164003, 94618007, 363418001, 700423003, 473419009, 1651000119109, 235966007, 208061000119101, 92264007, 254613007, 92547008, 92672004, 473418001, 143401000119106, 126861003, 126860002, 126859007, and/or 126862005. In some embodiments, the goal of one aspect of the present disclosure is to monitor a subject for a condition described by any combination of such SNOMED-CT values.

In some embodiments, the goal of one aspect of the present disclosure is gastric cancer. For instance, in some embodiments, the goal of one aspect of the present disclosure is to monitor a subject for a condition described by the SNOMED-CT value 126827000, 363349007, 428905002, 448882009, 255077007, 126824007, 408647009, 276809004, 276811008, 276810009, 447785000, 314961003, 187738005, 187742008, 187732006, 255078002, 187741001, 269460009, 269459004, 187740000, 187736009, 372014001, 94606003, 275108004, 473061005, and/or 367651003. In some embodiments, the goal of one aspect of the present disclosure is to monitor a subject for a condition described by any combination of such SNOMED-CT values.

In some embodiments, the goal of one aspect of the present disclosure is ureter cancer. For instance, in some embodiments, the goal of one aspect of the present disclosure is to monitor a subject for a condition described by the SNOMED-CT value 363458004.

In some embodiments, the goal of one aspect of the present disclosure is renal pelvis cancer. For instance, in some embodiments, the goal of one aspect of the present disclosure is to monitor a subject for a condition described by the SNOMED-CT value 363457009, 448215006, 188253000, 93985007, and/or 94514000. In some embodiments, the goal of one aspect of the present disclosure is to monitor a subject for a condition described by any combination of such SNOMED-CT values.

In some embodiments, the goal of one aspect of the present disclosure is overian cancer. For instance, in some embodiments, the goal of one aspect of the present disclosure is to monitor a subject for a condition described by the SNOMED-CT values 363443007, 369526004, 369533004, 254849005, 254869000, 369524001, 369525000, 369526004, 369527008, 369528003, 369559001, 369531002, 369532009, 369533004, 369534005, 369566000, 369567009, 93934004, 423627007, 94455000, 314191009, 254852002, 359987004, 416274001, 254856004, 254870004, 254872007, 254876005, 702368000, 314191009, 254852002, 254863004, 254871000, 254860001, 448376000, 422782004, 722685004, 722684000, 416274001, 369522002, 369529006, 716855006, 254856004, 359987004, 369523007, 369530001, 92669006, 424600001, 424486004, 423480004, and/or 429090009. In some embodiments, the goal of one aspect of the present disclosure is to monitor a subject for a condition described by any combination of such SNOMED-CT values.

In some embodiments, the goal of one aspect of the present disclosure is breast cancer. For instance, in some embodiments, the goal of one aspect of the present disclosure is to monitor a subject for a condition described by the SNOMED-CT value 415076002, 126926005, 254837009, 254838004, 286892007, 254841008, 286896005, 286897001, 286894008, 286893002, 286895009, 444604002, 372096000, 94297009, 706970001, 254843006, 417181009, 314955001, 278052009, 188050009, 372094002, 373080008, 373081007, 373082000, 373083005, 372064008, 372095001, 372137005, 278050001, 94544002, 708921005, 447782002, 372096000, 716593008, 427685000, 431396003, 408643008, 254840009, 278053004, 713609000, 403458008, 444604002, 444712000, 254839007, 427685000, 188153009, 373090000, 188155002, 373091001, 188152004, 373089009, 188154003, 373088001, 447782002, 188156001, 188151006, 188159008, 188153009, 188155002, 188147009, 188152004, 188154003, 93796005, 448449001, 94297009, 188168005, 188163001, 93884005, 448257000, 94401004, 716593008, 408643008, 278054005, 254840009, 188157005, 254844000, 444604002, 444712000, 721576006, 372092003, 373090000, 373091001, 373089009, 373088001, 93796005, 93884005, 93925009, 94012007, 722832009, 254839007, 708921005, 448731000124102, 92714001, 705089007, 189336000, 428748002, 286897001, 429087003, 408643008, 278054004, 93680004, 93924008, 109888004, 372064008, 93884005, 94012007, 94297009, 134405005, 126927001, 126937006, 126510002, 269497004, 92593006, 109890003, 109889007, 722523004, 721594000, 721595004, 92102001, 126929003, 126930008, 126936002, 126932000, 126934004, 126928006, 94836005, 126931007, 126933005, 92206006, 92652009, and/or 126939009. In some embodiments, the goal of one aspect of the present disclosure is to monitor a subject for a condition described by any combination of such SNOMED-CT values.

In some embodiments, the goal of one aspect of the present disclosure is triple negative breast cancer. For instance, in some embodiments, the goal of one aspect of the present disclosure is to monitor a subject for a condition described by the SNOMED-CT value 706970001.

In some embodiments, the goal of one aspect of the present disclosure is glioblastoma. For instance, in some embodiments, the goal of one aspect of the present disclosure is to monitor a subject for a condition described by the SNOMED-CT value 126961004, 428061005, 393563007, 147131000119101, 276828006, 276829003, and/or 63634009. In some embodiments, the goal of one aspect of the present disclosure is to monitor a subject for a condition described by any combination of such SNOMED-CT values.

In some embodiments, the goal of one aspect of the present disclosure is turcot syndrome. For instance, in some embodiments, the goal of one aspect of the present disclosure is to monitor a subject for a condition described by the SNOMED-CT value 61665008.

In some embodiments, the goal of one aspect of the present disclosure is small intestinal cancer. For instance, in some embodiments, the goal of one aspect of the present disclosure is to monitor a subject for a condition described by the SNOMED-CT values 363509000, 92750008, 94580002, 109837002, 424440001, 448664009, 449074003, 408644002, 709517003, 93775003, 93846004, and/or 94357009. In some embodiments, the goal of one aspect of the present disclosure is to monitor a subject for a condition described by any combination of such SNOMED-CT values.

In some embodiments, the goal of one aspect of the present disclosure is sebaceous gland adenomas. For instance, in some embodiments, the goal of one aspect of the present disclosure is to monitor a subject for a condition described by the SNOMED-CT value 403824007.

In some embodiments, the goal of one aspect of the present disclosure is kerataoacanthomas-muir-torre syndrome. For instance, in some embodiments, the goal of one aspect of the present disclosure is to monitor a subject for a condition described by the SNOMED-CT value 403824007. In some embodiments, the goal of one aspect of the present disclosure is synchronous or metachronous lynch syndrome. For instance, in some embodiments, the goal of one aspect of the present disclosure is to monitor a subject for a condition described by the SNOMED-CT value 315058005.

In some embodiments, the goal of one aspect of the present disclosure is polyposis. For instance, in some embodiments, the goal of one aspect of the present disclosure is to monitor a subject for a condition described by the SNOMED-CT value 301797007, 72900001, 87252009, 254590000, 254589009, 9273005, 709090007, and/or 138771000119100. In some embodiments, the goal of one aspect of the present disclosure is to monitor a subject for a condition described by any combination of such SNOMED-CT values.

In some embodiments, the goal of one aspect of the present disclosure is demoed tumor and/or desmoid tumor. For instance, in some embodiments, the goal of one aspect of the present disclosure is to monitor a subject for a condition described by the SNOMED-CT value 400153009.

In some embodiments, the goal of one aspect of the present disclosure is multifocal congenital hypertrophy of the retinal pigment epithelium (CHRPE). For instance, in some embodiments, the goal of one aspect of the present disclosure is to monitor a subject for a condition described by the SNOMED-CT value 232074003.

In some embodiments, the goal of one aspect of the present disclosure is bilateral CHRPE. For instance, in some embodiments, the goal of one aspect of the present disclosure is to monitor a subject for a condition described by the SNOMED-CT value 232074003.

In some embodiments, the goal of one aspect of the present disclosure is cribriform-morular variant. For instance, in some embodiments, the goal of one aspect of the present disclosure is to monitor a subject for a condition described by the SNOMED-CT values 255029007 or 708971008.

In some embodiments, the goal of one aspect of the present disclosure is papillary thyroid cancer. For instance, in some embodiments, the goal of one aspect of the present disclosure is to monitor a subject for a condition described by the SNOMED-CT values 255029007 or 708971008.

In some embodiments, the goal of one aspect of the present disclosure is hepablastoma. For instance, in some embodiments, the goal of one aspect of the present disclosure is to monitor a subject for a condition described by the SNOMED-CT value 109843000.

In some embodiments, the goal of one aspect of the present disclosure is adenomatous polyps. For instance, in some embodiments, the goal of one aspect of the present disclosure is to monitor a subject for a condition described by the SNOMED-CT value 82375006, 428054006, 29421000119105, 7401000119101, 60286009, 255196008, 128653004, 43233001, 36162004, 429047008, 430483006, 443157008 and/or 443734007. In some embodiments, the goal of one aspect of the present disclosure is to monitor a subject for a condition described by any combination of such SNOMED-CT values.

In some embodiments, the goal of one aspect of the present disclosure are cancer susceptibility gene marks. For instance, in some embodiments, the goal of one aspect of the present disclosure is to monitor a subject for a condition described by the SNOMED-CT values 445333001 or 85101000119100.

In some embodiments, the goal of one aspect of the present disclosure is kidney cancer. For instance, in some embodiments, the goal of one aspect of the present disclosure is to monitor a subject for a condition described by the SNOMED-CT values 363518003 or 415081006.

In some embodiments, the goal of one aspect of the present disclosure is leukemia. For instance, in some embodiments, the goal of one aspect of the present disclosure is to monitor a subject for a condition described by the SNOMED-CT value 93143009.

In some embodiments, the goal of one aspect of the present disclosure is bladder cancer. For instance, in some embodiments, the goal of one aspect of the present disclosure is to monitor a subject for a condition described by the SNOMED-CT value 399326009.

In some embodiments, the goal of one aspect of the present disclosure is melanoma. For instance, in some embodiments, the goal of one aspect of the present disclosure is to monitor a subject for a condition described by the SNOMED-CT values 372130007 or 372244006.

In some embodiments, the goal of one aspect of the present disclosure is prostate cancer. For instance, in some embodiments, the goal of one aspect of the present disclosure is to monitor a subject for a condition described by the SNOMED-CT value 254900004.

### EXAMPLE—CASE STUDIES OF IMPLEMENTATION OF THE PRESENT DISCLOSURE

Systems and methods of the present disclosure were shared with some predetermined practitioners in order to verify the monitoring of subjects for hereditary cancers. Data was collected and aggregated for approximately two to three months, depending on the practitioner, and ran against a plurality of filters for Lynch Syndrome, colon cancer, breast cancer, endometrial cancer, and/or ovarian cancer as well as if and/or when genetic testing is conducted.

TABLE 3

| | Number of | | | | | | Genetic | |
|---|---|---|---|---|---|---|---|---|
| Date | Patients | Lynch | Colon | Breast | Endometrial | Ovarian | Tested | Total |
| 4-Oct | 8 | 1 | | 1 | | | 2 | 2 |
| 5-Oct | 15 | | 1 | 1 | 2 | | 2 | 4 |
| 6-Oct | 5 | | 2 | | | 1 | 2 | 3 |
| 9-Oct | 16 | | 1 | 1 | | 2 | 3 | 4 |
| 10-Oct | 12 | | 1 | 1 | | | 2 | 2 |
| 11-Oct | 10 | 1 | | | | | 1 | 1 |
| 18-Oct | 9 | | 1 | 1 | | | 0 | 2 |
| 19-Oct | 14 | | 2 | | | 1 | 2 | 3 |
| 20-Oct | 11 | | | 1 | | | 1 | 1 |
| 23-Oct | 12 | 1 | | | | | | 1 |
| 24-Oct | 11 | 2 | 1 | 1 | | 1 | 2 | 5 |
| 25-Oct | 7 | 1 | | | | | | 1 |
| 26-Oct | 13 | | | | 1 | 1 | 0 | 2 |
| 27-Oct | 12 | | | | | | | 0 |
| 30-Oct | 15 | | | | | | | 0 |
| 31-Oct | 14 | | 1 | 1 | | 1 | | 3 |
| 1-Nov | 6 | | | | 1 | | | 1 |
| 2-Nov | 13 | 1 | | 1 | 3 | 1 | | 6 |
| 3-Nov | 9 | | | 1 | | 1 | 1 | 2 |
| 6-Nov | 16 | | 1 | | 1 | | | 2 |
| 7-Nov | 11 | 1 | 2 | | | | | 3 |
| 8-Nov | 7 | | 1 | | | | | 1 |
| 9-Nov | 15 | | 2 | 1 | | | | 3 |
| 10-Nov | 9 | | 1 | | 1 | 1 | | 3 |
| 13-Nov | 17 | | 2 | | | | | 2 |
| 14-Nov | 13 | | | | 2 | | | 2 |
| 15-Nov | 4 | | | | | | | 0 |
| 16-Nov | 12 | | 1 | | | | | 1 |
| 17-Nov | 7 | | 1 | | | | | 1 |
| 20-Nov | 15 | | | | | 2 | | 2 |
| 21-Nov | 15 | | 1 | | | | | 2 |
| 22-Nov | 15 | | | | | 1 | | 1 |
| 23-Nov | 0 | | | | | | | 0 |
| 24-Nov | 0 | | | | | | | 0 |
| 27-Nov | 0 | | | | | | | 0 |
| 28-Nov | 0 | | | | | | | 0 |
| 29-Nov | 0 | | | | | | | 0 |
| 30-Nov | 0 | | | | | | | 0 |
| 1-Dec | 0 | | | | | | | 0 |
| 4-Dec | 0 | | | | | | | 0 |
| 5-Dec | 0 | | | | | | | 0 |
| 6-Dec | 0 | | | | | | | 0 |
| 7-Dec | 0 | | | | | | | 0 |
| 8-Dec | 0 | | | | | | | 0 |
| 11-Dec | 0 | | | | | | | 0 |
| 12-Dec | 3 | | | | | | | 0 |
| 13-Dec | 12 | | | | | 1 | | 1 |
| 14-Dec | 8 | | | 1 | | | | 1 |
| 15-Dec | 9 | | | 1 | | 2 | | 3 |
| 18-Dec | 12 | | | | | 1 | | 1 |
| 19-Dec | 14 | | | | | | | 0 |
| 20-Dec | 4 | | | | | | | 0 |
| 21-Dec | 13 | | | 2 | | | | 2 |
| 22-Dec | 12 | | | | | | | 0 |
| 25-Dec | 0 | | | | | | | 0 |
| 26-Dec | 15 | | 1 | | | | | 1 |
| 27-Dec | 13 | | 1 | | | | | 1 |
| 28-Dec | 15 | | | 3 | | | | 3 |
| 29-Dec | 12 | | 1 | | | | | 1 |
| 30-Dec | 0 | | | | | | | 0 |
| 31-Dec | 0 | | | | | | | 0 |
| Totals | 510 | 8 | 25 | 18 | 11 | 17 | 18 | 80 |
| Proportion | | | | | | | 0.035294118 | 0.156862745 |
| of Patients | | | | | | | | |

TABLE 4

| | Number of | | | | | | Genetic | |
|---|---|---|---|---|---|---|---|---|
| Date | Patients | Lynch | Colon | Breast | Endometrial | Ovarian | Tested | Total |
| 26-Oct | 24 | | | 2 | | | 1 | 3 |
| 27-Oct | 12 | | | 1 | | 2 | | 3 |
| 30-Oct | 10 | | | 1 | | 2 | | 3 |
| 31-Oct | 13 | | | 5 | | | | 5 |
| 1-Nov | 1 | | | | | | | 0 |
| 2-Nov | 23 | | | 5 | | 1 | | 6 |
| 3-Nov | 23 | 1 | | 1 | | 1 | | 3 |
| 6-Nov | 7 | | | | 1 | 1 | | 2 |
| 7-Nov | 24 | | | 2 | | | | 2 |
| 8-Nov | 2 | | | | | | | 0 |
| 9-Nov | 23 | | 2 | 2 | | 1 | | 5 |
| 10-Nov | 20 | | | 2 | | 1 | | 3 |
| 13-Nov | 13 | | | 1 | | 2 | | 3 |
| 14-Nov | 14 | | | | | 2 | | 2 |
| 15-Nov | 0 | | | | | | | 0 |
| 16-Nov | 20 | | | 4 | | 1 | | 5 |
| 17-Nov | 17 | | | 1 | | 4 | | 5 |
| 20-Nov | 19 | | | 2 | | 2 | | 4 |
| 21-Nov | 16 | | | 2 | | 4 | | 6 |
| 22-Nov | 14 | | | | | 1 | | 1 |
| 23-Nov | 0 | | | | | | | 0 |
| 24-Nov | 0 | | | | | | | 0 |
| 27-Nov | 24 | | | 6 | | 2 | | 8 |
| 28-Nov | 15 | | 1 | 3 | | 1 | | 5 |
| 29-Nov | 5 | | | 2 | | | | 2 |
| 30-Nov | 26 | | | 2 | | 3 | | 5 |
| 1-Dec | 15 | | | 2 | | 1 | | 3 |
| 4-Dec | 15 | | | 1 | 1 | 1 | | 3 |
| 5-Dec | 22 | | | | 1 | 5 | | 6 |
| 6-Dec | 2 | | | | | | | 0 |
| 7-Dec | 21 | | | 2 | | 1 | | 3 |
| 8-Dec | 14 | | | 2 | | | | 2 |
| 11-Dec | 20 | | | | 2 | | | 2 |
| 12-Dec | 9 | | | 1 | 1 | 1 | | 3 |
| 13-Dec | 14 | | | | | 1 | | 1 |
| 14-Dec | 25 | | | 2 | 1 | | | 3 |
| 15-Dec | 19 | | | 2 | | 2 | | 4 |
| 18-Dec | 0 | | | | | | | 0 |
| 19-Dec | 0 | | | | | | | 0 |
| 20-Dec | 0 | | | | | | | 0 |
| 21-Dec | 0 | | | | | | | 0 |
| 22-Dec | 0 | | | | | | | 0 |
| 25-Dec | 0 | | | | | | | 0 |
| 26-Dec | 0 | | | | | | | 0 |
| 27-Dec | 18 | | | 1 | 1 | | | 2 |
| 28-Dec | 26 | | 1 | 4 | | | | 5 |
| 29-Dec | 18 | | | | | | | 0 |
| 30-Dec | 0 | | | | | | | 0 |
| 31-Dec | 0 | | | | | | | 0 |
| Totals | 603 | 0 | 0 | 9 | 0 | 4 | 1 | 118 |
| Proportion | | | | | | | | 0.19568823 |
| of Patients | | | | | | | | |

TABLE 5

| | Number of | | | | | | Genetic | |
|---|---|---|---|---|---|---|---|---|
| Date | Patients | Lynch | Colon | Breast | Endometrial | Ovarian | Tested | Total |
| 24-Oct | 9 | | 1 | | | | | 1 |
| 25-Oct | 10 | | | 3 | | 2 | | 5 |
| 26-Oct | 7 | 1 | 1 | | | | | 2 |
| 27-Oct | 7 | | | | | 1 | | 1 |
| 30-Oct | 7 | | | | | | | 2 |
| 31-Oct | 8 | | | 1 | 1 | 2 | | 4 |
| 1-Nov | 5 | | | 1 | | | | 1 |
| 2-Nov | 9 | | | | | | | 1 |
| 3-Nov | 11 | | | | | | | 2 |
| 6-Nov | 10 | | 1 | | | | | 1 |
| 7-Nov | 9 | | | 1 | 1 | | | 2 |
| 8-Nov | 9 | | 1 | | | | | 1 |
| 9-Nov | 10 | | | | | | | 0 |
| 10-Nov | 1 | | | | | | | 0 |
| 13-Nov | 10 | | 1 | 1 | | | | 2 |
| 14-Nov | 13 | | | 2 | | | | 2 |
| 15-Nov | 9 | | | 1 | | | | 1 |
| 16-Nov | 7 | | | 1 | 1 | | | 2 |
| 17-Nov | 0 | | | | | | | 0 |
| 20-Nov | 9 | | | 2 | | | | 2 |
| 21-Nov | 8 | | | 2 | | | | 2 |
| 22-Nov | 6 | | | 1 | | | | 1 |
| 23-Nov | 6 | | | | | | | 0 |
| 24-Nov | 0 | | | | | | | 0 |
| 27-Nov | 8 | | | 2 | | | | 2 |
| 28-Nov | 6 | | | | | | | 0 |
| 29-Nov | 10 | | | 4 | | | | 4 |
| 30-Nov | 8 | | 1 | | | | | 1 |
| 1-Dec | 7 | | | 1 | | | | 1 |
| 4-Dec | 7 | | 1 | 1 | | | | 2 |
| 5-Dec | 6 | | | 1 | | | | 1 |
| 6-Dec | 7 | | | 2 | | | | 2 |
| 7-Dec | 7 | | 1 | | | | | 1 |
| 8-Dec | 9 | | | 1 | | 1 | | 2 |
| 11-Dec | 6 | | 1 | 1 | | | | 2 |
| 12-Dec | 8 | | | 1 | | | | 1 |
| 13-Dec | 9 | | 1 | | 1 | | | 2 |
| 14-Dec | 11 | | 1 | | | 1 | | 2 |
| 15-Dec | 10 | | | 1 | | | | 1 |
| 18-Dec | 7 | | | 1 | | 1 | | 2 |
| 19-Dec | 10 | | 3 | 1 | | 1 | | 5 |
| 20-Dec | 10 | | | | | 2 | | 2 |
| 21-Dec | 6 | | | | | | | 0 |
| 22-Dec | 6 | | | 1 | | 1 | | 2 |
| 23-Dec | 2 | | | 1 | | | | 1 |
| 24-Dec | 0 | | | | | | | 0 |
| 25-Dec | 0 | | | | | | | 0 |
| 26-Dec | 0 | | | | | | | 0 |
| 27-Dec | 14 | | 1 | | | | | 1 |
| 28-Dec | 8 | | | 1 | | | | 1 |
| 29-Dec | 0 | | | | | | | 0 |
| 30-Dec | 0 | | | | | | | 0 |
| 31-Dec | 0 | | | | | | | 0 |
| Totals | 362 | 1 | 15 | 36 | 4 | 12 | 0 | 73 |
| Proportion of | | | | | | | | 0.201657459 |
| Patients | | | | | | | | |

TABLE 6

| | Number of | | | | | | Genetic | |
|---|---|---|---|---|---|---|---|---|
| Date | Patients | Lynch | Colon | Breast | Endometrial | Ovarian | Tested | Total |
| 13-Nov | 3 | | | | | | | 0 |
| 14-Nov | 5 | | | | | | | 0 |
| 15-Nov | 3 | | 1 | | | | | 1 |
| 16-Nov | 8 | | | 1 | 1 | | | 2 |
| 17-Nov | 7 | | 1 | | | | | 1 |
| 20-Nov | 0 | | | | | | | 0 |
| 21-Nov | 0 | | | | | | | 0 |
| 22-Nov | 0 | | | | | | | 0 |
| 23-Nov | 0 | | | | | | | 0 |
| 24-Nov | 0 | | | | | | | 0 |
| 27-Nov | 7 | | | 1 | | | | 1 |
| 28-Nov | 7 | | 1 | | | | | 1 |
| 29-Nov | 7 | | | 1 | | | | 1 |
| 30-Nov | 6 | | | | | | | 0 |
| 1-Dec | 5 | | 1 | | | 1 | | 2 |
| 4-Dec | 4 | | | | | | | 0 |
| 5-Dec | 4 | | | | | | | 0 |
| 6-Dec | 4 | | | 1 | | | | 1 |
| 7-Dec | 3 | | | | | | | 0 |
| 8-Dec | 3 | | | | | | | 0 |
| 11-Dec | 5 | | | 1 | | | | 1 |
| 12-Dec | 4 | | 1 | | | | | 1 |
| 13-Dec | 7 | | 1 | | | | | 1 |
| 14-Dec | 4 | | | 1 | | | | 1 |
| 15-Dec | 6 | | | | | | | 0 |
| 18-Dec | 4 | | | 2 | | | | 2 |
| 19-Dec | 7 | | | 1 | | | | 1 |
| 20-Dec | 8 | | | | | | | 0 |
| 21-Dec | 0 | | | | | | | 0 |
| 22-Dec | 0 | | | | | | | 0 |
| 25-Dec | 0 | | | | | | | 0 |
| 26-Dec | 2 | | | | | | | 2 |
| 27-Dec | 6 | | | 1 | | | | 1 |
| 28-Dec | 9 | | | | | | | 0 |
| 29-Dec | 5 | | | | | | | 0 |
| 30-Dec | 0 | | | | | | | 0 |
| 31-Dec | 0 | | | | | | | 0 |
| Totals | 143 | 0 | 6 | 10 | 1 | 1 | 0 | 20 |
| Proportion | | | | | | | | 0.13986014 |
| of Patients | | | | | | | | |

The results of Tables 3-5 are from studies where a clinician was guided through the present disclosure and presented with a listing of sample questions to ask patients prior to screening. Table 6 is a study where a clinician was not guided through the present disclosure and was not presented with a listing of sample questions to ask patients prior to screening, thus the study of Table 6 was conducted to verify the systems and methods of the present disclosure. As shown, patients of the clinician of Table 6 were still properly screened at a rate equivalent to the other case studies.

The combined case studies revealed a total of 291 patients were identified to have an actionable risk of a hereditary cancer from a total of 1618 patients, thus having a combined average of 18.0% detection. Previously, none of these patients would have been detected without meticulous data mining and time consumption. Of the first hundred patients scanned, twenty-two individuals were alerted for further genetic screening or testing. Of these twenty-two individuals, fifteen carried genes for hereditary cancer, yielding a 68.2% accuracy of detection.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in any combination of FIGS. 1 through 5 and/or described in FIG. 6 and/or FIG. 9. These program modules can be stored on a CD-ROM, DVD, magnetic disk storage product, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A method for determining whether a first human has an actionable risk for each of a plurality of hereditary cancers, comprising:
a) responsive to a request by a medical practitioner, obtaining an electronic medical record associated with the first human, via a computer system having a processor programmed to receive medical records, the electronic medical record of the first human comprising a plurality of data items, wherein the plurality of data items includes:
an age of the first human,
whether the first human has been diagnosed with a cancer, and
an indication of whether a second human has been afflicted with a cancer,
wherein the second human shares a first degree, second degree, or third degree consanguineous relationship with the first human;
b) for each respective hereditary cancer in the plurality of hereditary cancers, applying an algorithm to the plurality of data items, via a computer system having a processor programmed to perform the algorithm, wherein the algorithm runs the plurality of data items against a corresponding filter set in a plurality of filter sets, wherein
each respective filter set in the plurality of filter sets represents a different hereditary cancer in the plurality of hereditary cancers,
each respective filter set in the plurality of filter sets includes a respective filter that is configured to be fired at least when the plurality of data items indicates the first human has a familial risk factor for the corresponding hereditary cancer that satisfies a first threshold level of consanguinity,
each respective filter set in the plurality of filter sets is associated with a corresponding alert in a plurality of alerts, and
when a respective filter in the corresponding filter set is fired, the first human is deemed to have an actionable risk of the hereditary cancer represented by the corresponding filter set; and
c) communicating a report to the medical professional, via a computer system having a processor programmed to communicate reports, the report comprising the alert associated with each respective filter set in the plurality of filter sets that included a respective filter that was fired in response to applying the algorithm to the plurality of data items of the electronic medical record of the first human.

2. The method of claim 1, wherein
a first hereditary cancer in the plurality of hereditary cancers is a hereditary non-polyposis colorectal cancer (HNPCC) related cancer in a plurality of HNPCC related cancers, and
the corresponding filter set in the plurality of filter sets comprises:
a first filter that fires when the electronic medical record indicates (i) that the first human has not been diagnosed with colorectal cancer or endometrial cancer, (ii) that the second human shares a first degree consanguineous relationship with the first human, and (iii) that the second human was diagnosed with colorectal or endometrial cancer at an age less than a first predetermined age.

3. The method of claim 1, wherein
a first hereditary cancer in the plurality of hereditary cancers is a hereditary non-polyposis colorectal cancer (HNPCC) related cancer in a plurality of HNPCC related cancers, and
the corresponding filter set in the plurality of filter sets comprises:
a second filter that fires when the electronic medical record indicates (i) that the first human has not been diagnosed with colorectal cancer or endometrial cancer, (ii) that the second human shares a first degree consanguineous relationship with the first human, and (iii) that the second human has been diagnosed with colorectal or endometrial cancer and another HNPCC related cancer in the plurality of HNPCC related cancers other than colorectal or endometrial cancer.

4. The method of claim 1, wherein
a first hereditary cancer in the plurality of hereditary cancers is a hereditary non-polyposis colorectal cancer (HNPCC) related cancer in a plurality of HNPCC related cancers, and
the corresponding filter set in the plurality of filter sets comprises:
a third filter that fires when the electronic medical record indicates (i) that the first human has not been diagnosed with colorectal cancer or endometrial cancer, (ii) that the second human shares a first degree or second degree consanguineous relationship with the first human, (iii) that the second human has been diagnosed with an HNPCC related cancer in the plurality of HNPCC related cancers, (iv) that a third human shares a first degree or second degree consanguineous relationship with the first human, and (v) that the third human was diagnosed with an HNPCC related cancer in the plurality of HNPCC related cancers before a first predetermined age.

5. The method of claim 1, wherein
a first hereditary cancer in the plurality of hereditary cancers is a hereditary non-polyposis colorectal cancer (HNPCC) related cancer in a plurality of HNPCC related cancers, and
the corresponding filter set in the plurality of filter sets comprises:
a fourth filter that fires when the electronic medical record indicates (i) that the first human has not been diagnosed with colorectal cancer or endometrial cancer (ii) that the second human shares a first degree or second degree consanguineous relationship with the first human, (iii) that the second human has been diagnosed with an HNPCC related cancer in the plurality of HNPCC related cancers, (iv) that a third human shares a first degree or second consanguineous relationship with the first human, (v) that the third human has been diagnosed with an HNPCC related cancer in the plurality of HNPCC related cancers, (vi) that a fourth human shares a first degree or second degree consanguineous relationship with the first human, and (vii) that the fourth human has been diagnosed with an HNPCC related cancer in the plurality of HNPCC related cancers.

6. The method of claim 1, wherein
a first hereditary cancer in the plurality of hereditary cancers is a hereditary non-polyposis colorectal cancer (HNPCC) related cancer in a plurality of HNPCC related cancers, and
the corresponding filter set in the plurality of filter sets comprises:
a fifth filter that fires when the electronic medical record indicates (i) that the first human has not been diagnosed with colorectal cancer or endometrial cancer, (ii) that the second human shares a first degree consanguineous relationship with the first human, and (iii) that the second human has been diagnosed with polyposis.

7. The method of claim 1, wherein
a first hereditary cancer in the plurality of hereditary cancers is a hereditary non-polyposis colorectal cancer (HNPCC) related cancer in a plurality of HNPCC related cancers, and
the corresponding filter set in the plurality of filter sets comprises:
a sixth filter that fires when the electronic medical record indicates (i) that the first human has been diagnosed with colorectal cancer and (i) the first human is younger than a first predetermined age.

8. The method of claim 1, wherein
a first hereditary cancer in the plurality of hereditary cancers is a hereditary non-polyposis colorectal cancer (HNPCC) related cancer in a plurality of HNPCC related cancers, and
the corresponding filter set in the plurality of filter sets comprises:
a seventh filter that fires when the electronic medical record indicates that the first human has been diagnosed with endometrial cancer.

9. The method of claim 1, wherein
a first hereditary cancer in the plurality of hereditary cancers is a hereditary non-polyposis colorectal cancer (HNPCC) related cancer in a plurality of HNPCC related cancers, and
the corresponding filter set in the plurality of filter sets comprises:
an eighth filter that fires when the electronic medical record indicates (i) that the first human has been diagnosed with colorectal cancer or endometrial cancer and (ii) that the first human has been diagnosed with another HNPCC related cancer in the plurality of HNPCC related cancers.

10. The method of claim 1, wherein
a first hereditary cancer in the plurality of hereditary cancers is a hereditary non-polyposis colorectal cancer (HNPCC) related cancer in a plurality of HNPCC related cancers, and
the corresponding filter set in the plurality of filter sets comprises:
a ninth filter that fires when the electronic medical record indicates (i) that the first human has colorectal cancer or endometrial cancer, (ii) that the second human shares a first degree consanguineous relationship with the first human, and (iii) that the second human was diagnosed with an HNPCC related cancer in the plurality of HNPCC related cancers at an age less than a first predetermined age.

11. The method of claim 1, wherein
a first hereditary cancer in the plurality of hereditary cancers is a hereditary non-polyposis colorectal cancer (HNPCC) related cancer in a plurality of HNPCC related cancers, and
the corresponding filter set in the plurality of filter sets comprises:
a tenth filter that fires when the electronic medical record indicates (i) that the first human has colorectal cancer or endometrial cancer, (ii) that the second human shares a first degree or second degree consanguineous relationship with the first human, (iii) that the second human has been diagnosed with an HNPCC related cancer in the plurality of HNPCC related cancers, and (iv) that a third human shares a first degree or second degree consanguineous relationship with the first human, and (v) that the third human has been diagnosed with an HNPCC related cancer in the plurality of HNPCC related cancers.

12-18. (canceled)

19. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a first filter that fires when the electronic medical record indicates (i) that the first human has been diagnosed with breast cancer, (ii) that the second human shares a first degree, second degree, or third degree consanguineous relationship with the first human, and (iii) that the second human carries an HBOC related cancer susceptibility gene.

20. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a second filter that fires when the electronic medical record indicates that the first human has been diagnosed with ovarian cancer.

21. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a third filter that fires when the electronic medical record indicates (i) that the first human has been diagnosed with breast cancer, and (ii) that the first human no older than a second predetermined age when diagnosed with breast cancer.

22. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a fourth filter that fires when the electronic medical record indicates (i) that the first human has been diagnosed with breast cancer, (ii) that the breast cancer of the first human has been diagnosed as triple negative for estrogen receptor (ER), progesterone receptor (PR), and human epidermal growth factor receptor 2 (HER2), and (iii) that the first human was no older than a third predetermined age when diagnosed with triple negative breast cancer.

23. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a fifth filter that fires when the electronic medical record indicates that the first human has been diagnosed with breast cancer twice.

24. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a sixth filter that fires when the electronic medical record indicates (i) that the first human has been diagnosed with breast cancer, (ii) that the second human shares a first degree, a second degree, or a third degree consanguineous relationship with the first human, and (iii) that the second human was diagnosed with a breast cancer at an age of no more than a second predetermined age.

25. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a seventh filter that fires when the electronic medical record indicates (i) that the first human has been diagnosed with breast cancer, (ii) that the second human shares a first degree, a second degree, or a third degree consanguineous relationship with the first human, and (iii) that the second human has been diagnosed with ovarian cancer.

26. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
an eighth filter that fires when the electronic medical record indicates (i) that the first human has been diagnosed with breast cancer, (ii) that the second human shares a first degree, a second degree, or a third degree consanguineous relationship with the first human, and (iii) that the second human was diagnosed with a breast cancer at an age of no more than a second predetermined age.

27. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
an ninth fifth filter that fires when the electronic medical record indicates (i) that the first human has been diagnosed with breast cancer, (ii) that the second human shares a first degree, a second degree, or a third degree consanguineous relationship with the first human, (iii) that the second human was diagnosed with breast cancer or pancreatic cancer, (iv) that a third human shares a first degree, a second degree, or a third degree consanguineous relationship with the first human, and (v) that the third human was diagnosed with breast cancer or pancreatic cancer.

28. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a tenth filter that fires when the electronic medical record indicates (i) that the first human has been diagnosed with breast cancer, and (ii) that the first human has been diagnosed with pancreatic cancer.

29. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
an eleventh filter that fires when the electronic medical record indicates (i) that the first human has been diagnosed with breast cancer, and (ii) that the first human is from a population selected from the group consisting of Ashkenazi Jewish, Icelandic, Finnish, Dutch, Norwegian, Scottish, Irish, and African-American.

30. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a twelfth filter that fires when the electronic medical record indicates (i) that the first human has been diagnosed with breast cancer, and (ii) that the first human has been diagnosed with ovarian cancer.

31. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a thirteenth filter that fires when the electronic medical record indicates (i) that the first human has been diagnosed with breast cancer, and (ii) that the first human is male.

32. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a fourteenth filter that fires when the electronic medical record indicates (i) that the first human has not been diagnosed with breast cancer, (ii) that the second human shares a first degree, second degree, or third degree consanguineous relationship with the first human, and (iii) that the second human carries an HBOC related cancer susceptibility gene.

33. (canceled)

34. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a fifteenth filter that fires when the electronic medical record indicates (i) that the first human has not been diagnosed with breast cancer, (ii) that the second human shares a first degree, second degree, or third degree consanguineous relationship with the first human, and (iii) that the second human has been diagnosed with breast cancer twice.

35. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a sixteenth filter that fires when the electronic medical record indicates (i) that the first human does not have breast cancer, (ii) that the second human shares a first degree, second degree, or third degree consanguineous relationship with the first human, (iii) that the second human was diagnosed with breast cancer at an age of nor more than a second predetermined age, (iv) that a third human shares a first degree, second degree, or third degree consanguineous relationship with the first human, (v) that the third human is on the same side of the family as the second human with respect to the first human, and (vi) that the third human has been diagnosed with breast cancer.

36. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a seventeenth filter that fires when the electronic medical record indicates (i) that the first human does not have breast cancer, (ii) that the second human shares a first degree, second degree, or third degree consanguineous relationship with the first human, (iii) that the second human has been diagnosed with ovarian cancer.

37. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
an eighteenth filter that fires when the electronic medical record indicates (i) that the first human does not have breast cancer, (ii) that the second human shares a first degree, second degree, or third degree consanguineous relationship with the first human, (iii) that the second human has been diagnosed with male breast cancer.

38. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a nineteenth filter that fires when the electronic medical record indicates (i) that the first human does not have breast cancer, (ii) that the second human shares a first degree or second degree consanguineous relationship with the first human, (iii) that the second human was diagnosed with breast cancer at an age of no more than a fourth predetermined age.

39. The method of claim 1, wherein
a second hereditary cancer in the plurality of hereditary cancers is a hereditary breast and ovarian cancer syndrome (HBOC) related cancer in a plurality of HBOC related cancers, and
the corresponding filter set in the plurality of filter sets comprises:
a twentieth filter that fires when the electronic medical record indicates (i) that the first human does not have breast cancer, (ii) that there are three more humans, each sharing a first degree, second degree, or third degree consanguineous relationship with the first human, and (iii) that each of the three or more humans have been diagnosed with a condition selected from the group consisting of breast cancer, pancreatic cancer, prostate cancer, melanoma, sarcoma, adrenocortical carcinoma, brain tumor, leukemia, diffuse gastric cancer, colon cancer, endometrial cancer, thyroid cancer, kidney cancer, a dermatologic manifestation a macrocephaly, and hamartomatous polyps of GI tract.

40-48. (canceled)

49. A non-transitory computer readable storage medium for determining whether a first human has an actionable risk for each of a plurality of hereditary cancers, the non-transitory computer readable storage medium storing instructions, which when executed by a device comprising one or more processors, cause the one or more processors to perform a method comprising:
a) responsive to a request by a medical practitioner, obtaining an electronic medical record associated with the first human, via a computer system having a processor programmed to receive medical records, the electronic medical record of the first human comprising a plurality of data items, wherein the plurality of data items includes:
an age of the first human,
whether the first human has been diagnosed with a cancer, and
an indication of whether a second human has been afflicted with a cancer,
wherein the second human shares a first degree, second degree, or third degree consanguineous relationship with the first human;
b) for each respective hereditary cancer in the plurality of hereditary cancers, applying an algorithm to the plurality of data items, via a computer system having a processor programmed to perform the algorithm, wherein the algorithm runs the plurality of data items against a corresponding filter set in a plurality of filter sets, wherein
each respective filter set in the plurality of filter sets represents a different hereditary cancer in the plurality of hereditary cancers,
each respective filter set in the plurality of filter sets includes a respective filter that is configured to be fired at least when the plurality of data items indicates the first human has a familial risk factor for the corresponding hereditary cancer that satisfies a first threshold level of consanguinity,
each respective filter set in the plurality of filter sets is associated with a corresponding alert in a plurality of alerts, and
when a respective filter in the corresponding filter set is fired, the first human is deemed to have an actionable risk of the hereditary cancer represented by the corresponding filter set; and
c) communicating a report to the medical professional, via a computer system having a processor programmed to communicate reports, the report comprising the alert associated with each respective filter set in the plurality of filter sets that included a respective filter that was fired in response to applying the algorithm to the plurality of data items of the electronic medical record of the first human.

50. A computer system for determining whether a first human has an actionable risk for each of a plurality of hereditary cancers, comprising:
one or more processors;
memory; and
one or more programs stored in the memory for execution by the one or more processors, the one or more programs comprising instructions for performing a method comprising:
a) responsive to a request by a medical practitioner, obtaining an electronic medical record associated with the first human, via a computer system having a processor programmed to receive medical records, the electronic medical record of the first human comprising a plurality of data items, wherein the plurality of data items includes:
an age of the first human,
whether the first human has been diagnosed with a cancer, and
an indication of whether a second human has been afflicted with a cancer,
wherein the second human shares a first degree, second degree, or third degree consanguineous relationship with the first human;
b) for each respective hereditary cancer in the plurality of hereditary cancers, applying an algorithm to the plurality of data items, via a computer system having a processor programmed to perform the algorithm, wherein the algorithm runs the plurality of data items against a corresponding filter set in a plurality of filter sets, wherein
each respective filter set in the plurality of filter sets represents a different hereditary cancer in the plurality of hereditary cancers,
each respective filter set in the plurality of filter sets includes a respective filter that is configured to be fired at least when the plurality of data items indicates the first human has a familial risk factor for the corresponding hereditary cancer that satisfies a first threshold level of consanguinity,
each respective filter set in the plurality of filter sets is associated with a corresponding alert in a plurality of alerts, and
when a respective filter in the corresponding filter set is fired, the first human is deemed to have an actionable risk of the hereditary cancer represented by the corresponding filter set; and
c) communicating a report to the medical professional, via a computer system having a processor programmed to communicate reports, the report comprising the alert associated with each respective filter set in the plurality of filter sets that included a respective filter that was fired in response to applying the algorithm to the plurality of data items of the electronic medical record of the first human.
